(19) United States
(12) Patent Application Publication
(10) Pub. No.: US 2010/0016218 A1
(43) Pub. Date: Jan. 21, 2010

(54) CONTROLLED-RELEASE APOPTOSIS MODULATING COMPOSITIONS AND METHODS FOR THE TREATMENT OF OTIC DISORDERS

(75) Inventors: Jay Lichter, Rancho Santa Fe, CA (US); Benedikt Vollrath, San Diego, CA (US); Sergio G. Duron, San Diego, CA (US); Carl Lebel, Malibu, CA (US); Fabrice PIU, San Diego, CA (US); Qiang Ye, San Diego, CA (US); Luis A. Dellamary, San Marcos, CA (US); Michael Christopher Scaife, Los Altos, CA (US); Andrew M. Trammel, Olathe, KS (US); Jeffrey P. Harris, La Jolla, CA (US)

Correspondence Address: WILSON, SONSINI, GOODRICH & ROSATI, 650 PAGE MILL ROAD, PALO ALTO, CA, 94304-1050, US

(73) Assignees: OTONOMY, INC., San Diego, CA (US); THE REGENTS OF THE UNIVERSITY OF CALIFORNIA, Oakland, CA (US)

(21) Appl. No.: 12/500,486

(22) Filed: Jul. 9, 2009

Related U.S. Application Data
(60) Provisional application No. 61/080,583, filed on Jul. 14, 2008.
(60) Provisional application No. 61/082,450, filed on Jul. 21, 2008.
(60) Provisional application No. 61/094,384, filed on Sep. 4, 2008.
(60) Provisional application No. 61/101,112, filed on Sep. 29, 2008.
(60) Provisional application No. 61/110,511, filed on Oct. 31, 2008.
(60) Provisional application No. 61/140,033, filed on Dec. 22, 2008.
(60) Provisional application No. 61/164,841, filed on Mar. 30, 2009.

(30) Foreign Application Priority Data
Apr. 24, 2009 (GB) 0907065.7

(51) Int. Cl. A61K 38/22; A61K 38/16; A61K 47/32; A61P 27/02

(52) U.S. Cl. 514/8; 514/12; 514/772.1

(57) ABSTRACT

Disclosed herein are compositions and methods for the treatment of otic disorders with anti-apoptotic agent or pro-apoptotic agent compositions and compositions administered locally to an individual afflicted with an otic disorder, through direct application of these compositions and compositions onto or via perfusion into the targeted auris structure(s).

### CROSS-REFERENCE

This application claims the benefit of U.S. Provisional Application No. 61/080,583, filed 14 Jul. 2008; U.S. Provisional Application No. 61/082,450, filed 21 Jul. 2008; U.S. Provisional Application No. 61/094,384, filed 4 Sep. 2008; U.S. Provisional Application No. 61/101,112, filed 29 Sep. 2008; U.S. Provisional Application No. 61/110,511, filed 31 Oct. 2008; U.S. Provisional Application No. 61/140,033, filed 22 Dec. 2008; U.S. Provisional Application No. 61,164,841, filed 30 Mar. 2009; and UK Patent Application No. 09 07065.7, filed 24 Apr. 2009; all of which are incorporated herein by reference in their entirety.

### BACKGROUND OF THE INVENTION

Vertebrates have a pair of ears, placed symmetrically on opposite sides of the head. The ear serves as both the sense organ that detects sound and the organ that maintains balance and body position. The ear is generally divided into three portions: the outer ear, auris media (or middle ear) and the auris interna (or inner ear).

### SUMMARY OF THE INVENTION

Described herein, in certain embodiments, are compositions, compositions, manufacturing methods, therapeutic methods, uses, kits, and delivery devices for the controlled-release of an anti-apoptotic agent or pro-apoptotic agent to at least one structure or region of the ear. Disclosed herein, in certain embodiments, are controlled-release compositions for delivering an anti-apoptotic agent or pro-apoptotic agent to the ear. In some embodiments, the target portion of the ear is the middle ear (or auris media). In some embodiments, the target portion of the ear is the inner ear (or auris interna). In other embodiments, the target portion of the ear is both the auris media and the auris interna. In some embodiments, the controlled-release compositions further comprise a rapid or immediate release component for delivering an anti-apoptotic agent or pro-apoptotic agent to the targeted auris structure. All compositions comprise excipients that are auris-acceptable.

Also disclosed herein, in certain embodiments, are compositions and devices for the treatment of otic disorders, said compositions and devices comprising an anti-apoptotic agent or pro-apoptotic agent. Further disclosed herein, in certain embodiments, are methods for the treatment of otic disorders by administration of a controlled-release composition comprising an anti-apoptotic agent or pro-apoptotic agent to an individual in need thereof. In some embodiments, the otic disorder is excitotoxicity, ototoxicity, presbycusis, or combinations thereof.

Also disclosed herein, in certain embodiments, are compositions and devices for selectively inducing apoptosis in a target portion of the ear, including the auris media (including substructures therein) and/or the auris interna (including substructures therein, e.g., the cochlea), said compositions and devices comprising a pro-apoptotic agent. Also disclosed herein, in certain embodiments, are compositions and devices for selectively preventing apoptosis in a target portion of the ear, including the auris media (including substructures therein) and/or the auris interna (including substructures therein, e.g., the cochlea), said compositions and devices comprising an anti-apoptotic agent.

The auris compositions and therapeutic methods described herein have numerous advantages that overcome the previously-unrecognized limitations of compositions and therapeutic methods described in prior art.

### Sterility

The environment of the inner ear is an isolated environment. The endolymph and the perilymph are static fluids and are not in contiguous contact with the circulatory system. The blood-labyrinth-barrier (BLB), which includes a blood-endolymph barrier and a blood-perilymph barrier, consists of tight junctions between specialized epithelial cells in the labyrinth spaces (i.e., the vestibular and cochlear spaces). The presence of the BLB limits delivery of active agents (e.g., anti-apoptotic agent or pro-apoptotic agents) to the isolated microenvironment of the inner ear. Auris hair cells are bathed in endolymphatic or perilymphatic fluids and cochlear recycling of potassium ions is important for hair cell function. When the inner ear is infected, there is an influx of leukocytes and/or immunoglobulins (e.g. in response to a microbial infection) into the endolymph and/or the perilymph and the ionic composition of inner ear fluids is upset by the influx of leukocytes and/or immunoglobulins. In certain instances, a change in the ionic composition of inner ear fluids results in hearing loss, loss of balance and/or ossification of auditory structures. In certain instances, trace amounts of pyrogens and/or microbes trigger infections and related physiological changes in the isolated microenvironment of the inner ear.

Due to the susceptibility of the inner ear to infections, auris compositions require a level of sterility that has not been recognized hitherto in prior art. Provided herein are auris compositions that are sterilized with stringent sterility requirements and are suitable for administration to the middle and/or inner ear. In some embodiments, the auris compatible compositions described herein are substantially free of pyrogens and/or microbes.

Compatibility with Inner Ear Environment

Described herein are otic compositions with an ionic balance that is compatible with the perilymph and/or the endolymph and does not cause any change in cochlear potential. In specific embodiments, osmolarity/osmolality of the present compositions is adjusted, for example, by the use of appropriate salt concentrations (e.g., concentration of sodium salts) or the use of tonicity agents that render the compositions endolymph-compatible and/or perilymph-compatible (i.e. isotonic with the endolymph and/or perilymph). In some instances, the endolymph-compatible and/or perilymph-compatible compositions described herein cause minimal disturbance to the environment of the inner ear and cause minimum discomfort (e.g., vertigo) to a subject (e.g., a human) upon administration. Further, the compositions comprise polymers that are biodegradable and/or dispersible, and/or otherwise non-toxic to the inner ear environment. In some embodiments, the compositions described herein are free of preservatives and cause minimal disturbance (e.g., change in pH or osmolarity, irritation) in auditory structures. In some embodiments, the compositions described herein comprise antioxidants that are non-irritating and/or non-toxic to otic structures.

### Dosing Frequency

The current standard of care for auris compositions requires multiple administrations of drops or injections (e.g. intratympanic injections) over several days (e.g., up to two weeks), including schedules of receiving multiple injections per day. In some embodiments, auris compositions described herein are controlled-release compositions and are administered at reduced dosing frequency compared to the current standard of care. In certain instances, when an auris composition is administered via intratympanic injection, a reduced frequency of administration alleviates discomfort caused by multiple intratympanic injections in individuals undergoing treatment for a middle and/or inner ear disease, disorder or condition. In certain instances, a reduced frequency of administration of intratympanic injections reduces the risk of permanent damage (e.g., perforation) to the tympanic membrane. The compositions described herein provide a constant, sustained, extended, delayed or pulsatile rate of release of an active agent into the inner ear environment and thus avoid any variability in drug exposure in treatment of otic disorders.

### Therapeutic Index

Auris compositions described herein are administered into the ear canal, or in the vestibule of the ear. In some embodiments, access to the vestibular and cochlear apparatus occurs through the auris media (e.g., the round window membrane, the oval window/stapes footplate, the annular ligament and through the otic capsule/temporal bone). Otic administration of the compositions described herein avoids toxicity associated with systemic administration (e.g., hepatotoxicity, cardiotoxicity, gastrointestinal side effects, renal toxicity) of the active agents. In some instances, localized administration in the ear allows an active agent to reach a target (e.g., the inner ear) in the absence of systemic accumulation of the active agent. In some instances, local administration to the ear provides a higher therapeutic index for an active agent that would otherwise have dose-limiting systemic toxicity.

Prevention of Drainage into Eustachian Tube

In some instances, a disadvantage of liquid compositions is their propensity to drip into the eustachian tube and cause rapid clearance of the composition from the inner ear. Provided herein, in certain embodiments, are auris compositions comprising polymers that gel at body temperature and remain in contact with the target auditory surfaces (e.g., the round window) for extended periods of time. In some embodiments, the compositions further comprise a mucoadhesive that allows the compositions to adhere to otic mucosal surfaces. In some instances, the auris compositions described herein avoid attenuation of therapeutic benefit due to drainage or leakage of active agents via the eustachian tube.

### DESCRIPTION OF CERTAIN EMBODIMENTS

Described herein, in certain embodiments, are controlled-release compositions and devices for treating otic disorders comprising a therapeutically-effective amount of an anti-apoptotic agent or pro-apoptotic agent, a controlled-release auris-acceptable excipient and an auris-acceptable vehicle.

Also described herein, in certain embodiments, are compositions and devices for selectively inducing apoptosis in a target portion of the ear, including the auris media (including substructures therein) and/or the auris interna (including substructures therein, e.g., the cochlea), said compositions and devices comprising an pro-apoptotic agent. Also disclosed herein, in certain embodiments, are compositions and devices for selectively preventing apoptosis in a target portion of the ear, including the auris media (including substructures therein) and/or the auris interna (including substructures therein, e.g., the cochlea), said compositions and devices comprising an anti-apoptotic agent.

In one aspect of the compositions and devices described herein, the controlled-release auris-acceptable excipient is chosen from an auris-acceptable polymer, an auris-acceptable viscosity enhancing agent, an auris-acceptable gel, an auris-acceptable paint, an auris-acceptable foam, an auris-acceptable microsphere or microparticle, an auris-acceptable hydrogel, an auris-acceptable in situ forming spongy material, an auris-acceptable actinic radiation curable gel, an auris-acceptable liposome, an auris-acceptable nanocapsule or nanosphere, an auris-acceptable thermoreversible gel or combinations thereof. In further embodiments, the auris-acceptable viscosity enhancing agent is a cellulose, a cellulose ether, alginate, polyvinylpyrrolidone, a gum, a cellulosic polymer or combinations thereof. In yet another embodiment, the auris-acceptable viscosity enhancing agent is present in an amount sufficient to provide a viscosity of between about 1000 to about 1,000,000 centipoise. In still another aspect, the auris-acceptable viscosity enhancing agent is present in an amount sufficient to provide a viscosity of between about 50,000 to about 1,000,000 centipoise.

In some embodiments, the compositions disclosed herein are formulated for a pH that ensures that they are compatible with the targeted auris structure. In some embodiments, the compositions disclosed herein are formulated for a practical osmolality and/or osmolarity that ensures that homeostasis of the target auris structure is maintained. A perilymph-suitable osmolarity/osmolality is a practical osmolarity/osmolality that maintains the homeostasis of the target auris structure during administration of the pharmaceutical compositions described herein.

For example, the osmolarity of the perilymph is between about 270-300 mOsm/L and the compositions described herein are optionally formulated to provide a practical osmolarity of about 150 to about 1000 mOsm/L. In certain embodiments, the compositions described herein provide a practical osmolarity within about 150 to about 500 mOsm/L at the target site of action (e.g., the inner ear and/or the perilymph and/or the endolymph). In certain embodiments, the compositions described herein provide a practical osmolarity within about 200 to about 400 mOsm/L at the target site of action (e.g., the inner ear and/or the perilymph and/or the endolymph). In certain embodiments, the compositions described herein provide a practical osmolarity within about 250 to about 320 mOsm/L at the target site of action (e.g., the inner ear and/or the perilymph and/or the endolymph). In certain embodiments, the compositions described herein provide a perilymph-suitable osmolarity within about 150 to about 500 mOsm/L, about 200 to about 400 mOsm/L or about 250 to about 320 mOsm/L at the target site of action (e.g., the inner ear and/or the perilymph and/or the endolymph). In certain embodiments, the compositions described herein provide a perilymph-suitable osmolality within about 150 to about 500 mOsm/kg, about 200 to about 400 mOsm/kg or about 250 to about 320 mOsm/kg at the target site of action (e.g., the inner ear and/or the perilymph and/or the endolymph). Similarly, the pH of the perilymph is about 7.2-7.4, and the pH of the present compositions is formulated (e.g., with the use of buffers) to provide a perilymph-suitable pH of about 5.5 to about 9.0, about 6.0 to about 8.0 or about 7.0 to about 7.6. In certain embodiments, the pH of the compositions is within about 6.0 to about 7.6. In certain instances, the pH of the endolymph is about 7.2-7.9, and the pH of the present compositions is formulated (e.g., with the use of buffers) to be within about 5.5 to about 9.0, within about 6.5 to about 8.0 or within about 7.0 to about 7.6.

In some aspects, the controlled-release auris-acceptable excipient is biodegradable and/or bioeliminated (e.g., degraded and/or eliminated through urine, feces or other routes of elimination). In another aspect, the controlled-release composition further comprises an auris-acceptable mucoadhesive, an auris-acceptable penetration enhancer or an auris-acceptable bioadhesive.

In one aspect, the controlled-release composition is delivered using a drug delivery device, which is a needle and syringe, a pump, a microinjection device, and in situ forming spongy material or combinations thereof. In some embodiments, the anti-apoptotic agent or pro-apoptotic agent of the controlled-release composition has limited or no systemic release, is toxic when administered systemically, has poor pK characteristics, or combinations thereof.

In further aspects, the anti-apoptotic agent is Akt, an agonist of Akt, or a homologue or mimic thereof; Bre, an agonist of Bre, or a homologue or mimic thereof; erythropoietin, an agonist of erythropoietin, or a homologue or mimic thereof; fortilin, an agonist of fortilin, or a homologue or mimic thereof; a recombinant FNK protein (e.g., a FNK-TAT fusion protein); ghrelin, an agonist of ghrelin, or a homologue or mimic thereof; IAP (inhibitor of apoptosis protein), an agonist of IAP, or a homologue or mimic thereof; a PI3 kinase, an agonist of a PI3 kinase, or a homologue or mimic thereof; sirtuin, an agonist of sirtuin, or a homologue or mimic thereof; an inhibitor of the MAPK/JNK signaling cascade; an inhibitor of a member of the Bcl-2 family; an inhibitor of Fas; an inhibitor of NF-kB; an inhibitor of P38; an inhibitor of Ca²⁺ channels; an inhibitor of HO-1; an inhibitor of a caspase; an inhibitor of a calpain; p53; an inhibitor of the Src family of protein kinases; a trefoil factor, an agonist of a trefoil factor, or a homologue or mimic thereof; an Hsp, an agonist of an Hsp, or a homologue or mimic thereof; an apolipoprotein, an agonist of an apolipoprotein, or a homologue or mimic thereof; or combinations thereof.

In further aspects, the pro-apoptotic agent is an antagonist of Akt; an antagonist of Bre; an antagonist of erythropoietin; an antagonist of fortilin; an antagonist of ghrelin; an antagonist of IAP (inhibitor of apoptosis protein); an antagonist of a PI3 kinase; an antagonist of sirtuin; an agonist of the MAPK/JNK signaling cascade; an agonist of a member of the Bcl-2 family; an agonist of Fas; an inhibitor of NF-kB, an agonist of P38, an agonist of Ca²⁺ channels, an agonist of HO-1, an agonist of a caspase, an agonist of a calpain, p53, an agonist of the Src family of protein kinases, or combinations thereof.

In another aspect, the anti-apoptotic agent or pro-apoptotic agent is a salt or prodrug of the anti-apoptotic agent or pro-apoptotic agent. In other aspects, the anti-apoptotic agent or pro-apoptotic agent is minocycline; SB-203580 (4-(4-Fluorophenyl)-2-(4-methylsulfinyl phenyl)-5-(4-pyridyl) 1H-imidazole); PD 169316 (4-(4-Fluorophenyl)-2-(4-nitrophenyl)-5-(4-pyridyl)-1H-imidazole); SB 202190 (4-(4-Fluorophenyl)-2-(4-hydroxyphenyl)-5-(4-pyridyl) 1H-imidazole); RWJ 67657 (4-[4-(4-fluorophenyl)-1-(3-phenylpropyl)-5-(4-pyridinyl)-1H-imidazol-2-yl]-3-butyn-1-ol); SB 220025 (5-(2-Amino-4-pyrimidinyl)-4-(4-fluorophenyl)-1-(4-piperidinlyl)imidazole); D-JNKI-1 ((D)-hJIP₁₇₅₋₁₅₇-DPro-DPro-(D)-HIV-TAT₅₇₋₄₈); AM-111 (Auris); SP600125 (anthra[1,9-cd]pyrazol-6(2H)-one); JNK Inhibitor I ((L)-HIV-TAT₄₈₋₅₇-PP-JBD₂₀); JNK Inhibitor III ((L)-HIV-TAT₄₇₋₅₇-gaba-c-Junδ₃₃₋₅₇); AS601245 (1,3-benzothiazol-2-yl (2-[[2-(3-pyridinyl)ethyl]amino]-4 pyrimidinyl) acetonitrile); JNK Inhibitor VI (H₂N-RPKRPTTLNLF-NH₂); JNK Inhibitor VIII (N-(4-Amino-5-cyano-6-ethoxypyridin-2-yl)-2-(2,5-dimethoxyphenyl)acetamide); JNK Inhibitor IX (N-(3-Cyano-4,5,6,7-tetrahydro-1-benzothien-2-yl)-1-naphthamide); dicumarol (3,3′-Methylenebis(4-hydroxycoumarin)); SC-236 (4-[5-(4-chlorophenyl)-3-(trifluoromethyl)-1H-pyrazol-1-yl]benzene-sulfonamide); CEP-1347 (Cephalon); CEP-11004 (Cephalon); an artificial protein comprising at least a portion of a Bcl-2 polypeptide; a recombinant FNK; V5 (also known as Bax inhibitor peptide V5); Bax channel blocker ((±)-1-(3,6-Dibromocarbazol-9-yl)-3-piperazin-1-yl-propan-2-ol); Bax inhibiting peptide P5 (also known as Bax inhibitor peptide P5); Kp7-6; FAIM(S) (Fas apoptosis inhibitory molecule-short); FAIM(L) (Fas apoptosis inhibitory molecule-long); Fas:Fc; FAP-1; NOK2; F2051; F1926; F2928; ZB4; Fas M3 mAb; EGF; 740 Y—P; SC 3036 (KKHTDDGYMPMSPGVA); PI 3-kinase Activator (Santa Cruz Biotechnology, Inc.); Pam₃Cys ((S)-(2,3-bis(palmitoyloxy)-(2RS)-propyl)-N-palmitoyl-(R)-Cys-(S)-Ser(S)-Lys4-OH, trihydrochloride); Act1 (NF-kB activator 1); an anti-IkB antibody; Acetyl-11-keto-b-Boswellic Acid; Andrographolide; Caffeic Acid Phenethyl Ester (CAPE); Gliotoxin; Isohelenin; NEMO-Binding Domain Binding Peptide (DRQIKIWFQNRRMKWKKTALDWSWLQTE); NF-kB Activation Inhibitor (6-Amino-4-(4-phenoxyphenylethylamino)quinazoline); NF-kB Activation Inhibitor II (4-Methyl-N1-(3-phenylpropyl)benzene-1,2-diamine); NF-kB Activation Inhibitor III (3-Chloro-4-nitro-N-(5-nitro-2-thiazolyl)-benzamide); NF-kB Activation Inhibitor IV ((E)-2-Fluoro-4′-methoxystilbene); NF-kB Activation Inhibitor V (5-Hydroxy-(2,6-diisopropylphenyl)-1H-isoindole-1,3-dione); NF-kB SN50 (AAVALLPAVLLALLAPVQRKRQKLMP); Oridonin; Parthenolide; PPM-18 (2-Benzoylamino-1,4-naphthoquinone); Ro106-9920; Sulfasalazine; TIRAP Inhibitor Peptide (RQIKIWFNRRMKWKKLQLRDAAPGGAIVS); Withaferin A; Wogonin; BAY 11-7082 ((E)3-[(4-Methylphenyl)sulfonyl]-2-propenenitrile); BAY 11-7085 ((E)₃-[(4-t-Butylphenyl)sulfonyl]-2-propenenitrile); (E)-Capsaicin; Aurothiomalate (ATM or AuTM); Evodiamine; Hypoestoxide; IKK Inhibitor III (BMS-345541); IKK Inhibitor VII; IKK Inhibitor X; IKK Inhibitor II; IKK-2 Inhibitor IV; IKK-2 Inhibitor V; IKK-2 Inhibitor VI; IKK-2 Inhibitor (SC-514); IkB Kinase Inhibitor Peptide; IKK-3 Inhibitor IX; ARRY-797 (Array BioPharma); SB-220025 (5-(2-Amino-4-pyrimidinyl)-4-(4-fluorophenyl)-1-(4-piperidinlyl)imidazole); SB-239063 (trans-4-[4-(4-Fluorophenyl)-5-(2-methoxy-4-pyrimidinyl)-1H-imidazol-1-yl]cyclohexanol); SB-202190 (4-(4-Fluorophenyl)-2-(4-hydroxyphenyl)-5-(4-pyridyl) 1H-imidazole); JX-401 (-[2-Methoxy-4-(methylthio)benzoyl]-4-(phenylmethyl)piperidine); PD-169316 (4-(4-Fluorophenyl)-2-(4-nitrophenyl)-5-(4-pyridyl)-1H-imidazole); SKF-86002 (6-(4-Fluorophenyl)-2,3-dihydro-5-(4-pyridinyl)imidazo[2,1-b]thiazole dihydrochloride); SB-200646 (N-(1-Methyl-1H-indol-5-yl)-N′-3-pyridinylurea); CMPD-1 (2′-Fluoro-N-(4-hydroxyphenyl)-[1,1′-biphenyl]-4-butanamide); EO-1428 ((2-Methylphenyl)-[4-[(2-amino-4-bromophenyl)amino]-2-chlorophenyl]methanone); SB-253080 (4-[5-(4-Fluorophenyl)-2-[4-(methylsulfonyl)phenyl]-1H-imidazol-4-yl]pyridine); SD-169 (1H-Indole-5-carboxamide); SB-203580 (4-(4-Fluorophenyl)-2-(4-methylsulfinyl phenyl)-5-(4-pyridyl) 1H-imidazole); TZP-101 (Tranzyme Pharma); TZP-102 (Tranzyme Pharma); GHRP-6 (growth hormone-releasing peptide-6); GHRP-2 (growth hormone-releasing peptide-2); EX-1314 (Elixir Pharmaceuticals); MK-677 (Merck); L-692,429 (Butanamide, 3-amino-3-methyl-N-(2,3,4,5-tetrahydro-2-oxo-1-((2′-(1H-tetrazol-5-yl)(1,1′-biphenyl)-4-yl)methyl)-1H-1-benzazepin-3-yl)-, (R)—); EP1572 (Aib-DTrp-DgTrp-CHO); diltiazem; metabolites of diltiazem; BRE (Brain and Reproductive organ-Expressed protein); verapamil; nimodipine; diltiazem; omega-conotoxin; GVIA; amlodipine; felodipine; lacidipine; mibefradil; NPPB (5-Nitro-2-(3-phenylpropylamino)benzoic Acid); flunarizine; erythropoietin; piperine; hemin; brazilin; z-VAD-FMK (Benzyloxycarbonyl-Val-Ala-Asp(OMe)-fluoromethylketone); z-LEHD-FMK (benzyloxycarbonyl-Leu-Glu(OMe)-His-Asp(OMe)-fluoromethylketone); B-D-FMK (boc-aspartyl(Ome)-fluoromethylketone); Ac-LEHD-CHO (N-acetyl-Leu-Glu-His-Asp-CHO); Ac-IETD-CHO(N-acetyl-Ile-Glu-Thr-Asp-CHO); z-IETD-FMK (benzyloxycarbonyl-Ile-Glu(OMe)-Thr-Asp(OMe)-fluoromethy lketone); FAM-LEHD-FMK (benzyloxycarbonyl Leu-Glu-His-Asp-fluoromethyl ketone); FAM-LETD-FMK (benzyloxycarbonyl Leu-Glu-Thr-Asp-fluoromethyl ketone); Q-VD-OPH (Quinoline-Val-Asp-CH₂—O-Ph); XIAP; cIAP-1; cIAP-2; ML-IAP; ILP-2; NAIP; Survivin; Bruce; IAPL-3; fortilin; leupeptine; PD-150606 (3-(4-Iodophenyl)-2-mercapto-(Z)-2-propenoic acid); MDL-28170 (Z-Val-Phe-CHO); calpeptin; acetyl-calpastatin; MG 132 (N-[(phenylmethoxy)carbonyl]-L-leucyl-N-[(1S)-1-formyl-3-methylbutyl]-L-leucinamide); MYODUR; BN 82270 (Ipsen); BN 2204 (Ipsen); AHLi-11 (Quark Pharmaceuticals), an mdm2 protein, pifithrin-α(1-(4-Methylphenyl)-2-(4,5,6,7-tetrahydro-2-imino-3 (2H)-benzothiazolyl)ethanone); trans-stilbene; cis-stilbene; resveratrol; piceatannol; rhapontin; deoxyrhapontin; butein; chalcon; isoliquirtigen; butein; 4,2′,4′-trihydroxychalcone; 3,4,2′,4′,6′-pentahydroxychalcone; flavone; morin; fisetin; luteolin; quercetin; kaempferol; apigenin; gossypetin; myricetin; 6-hydroxyapigenin; 5-hydroxyflavone; 5,7,3′,4′,5′-pentahydroxyflavone; 3,7,3′,4′,5′-pentahydroxyflavone; 3,6,3′,4′-tetrahydroxyflavone; 7,3′,4′,5′-tetrahydroxyflavone; 3,6,2′,4′-tetrahydroxyflavone; 7,4′-dihydroxyflavone; 7,8,3′,4′-tetrahydroxyflavone; 3,6,2′,3′-tetrahydroxyflavone; 4′-hydroxyflavone; 5-hydroxyflavone; 5,4′-dihydroxyflavone; 5,7-dihydroxyflavone; daidzein; genistein; naringenin; flavanone; 3,5,7,3′,4′-pentahydroxyflavanone; pelargonidin chloride; cyanidin chloride; delphinidin chloride; (−)-epicatechin (Hydroxy Sites: 3,5,7,3′,4′); (−)-catechin (Hydroxy Sites: 3,5,7,3′,4′); (−)-gallocatechin (Hydroxy Sites: 3,5,7,3′,4′,5′) (+)-catechin (Hydroxy Sites: 3,5,7,3′,4′); (+)-epicatechin (Hydroxy Sites: 3,5,7,3′,4′); Hinokitiol (b-Thujaplicin; 2-hydroxy-4-isopropyl-2,4,6-cycloheptatrien-1-one); L-(+)-Ergothioneine ((S)-a-Carboxy-2,3-dihydro-N,N,N-trimethyl-2-thioxo-1H-imidazole4-ethanaminium inner salt); Caffeic Acid Phenyl Ester; MC1-186 (3-Methyl-1-phenyl-2-pyrazolin-5-one); HBED (N,N′-Di-(2-hydroxybenzyl)ethylenediamine-N,N′-diacetic acidH2O); Ambroxol (trans-4-(2-Amino-3,5-dibromobenzylamino)cyclohexane-HCl; and U-83836E ((−)-2-((4-(2,6-di-1-Pyrrolidinyl-4-pyrimidinyl)-1-piperzainyl)methyl)-3,4-dihydro-2,5,7,8-tetramethyl-2H-1-benzopyran-6-ol2HCl); β-1′-5-methyl-nicotinamide-2′-deoxyribose; β-D-1′-5-methyl-nico-tinamide-2′-deoxyribofuranoside; β-1′-4,5-dimethyl-nicotinamide-2′-de-oxyribose; β-D-1′-4,5-dimethyl-nicotinamide-2′-deoxyribofuranoside; 1-Naphthyl PP1(1-(1,1-Dimethylethyl)-3-(1-naphthalenyl)-1H-pyrazolo[3,4-d]pyrimidin-4-amine); Lavendustin A (5-[[(2,5-Dihydroxyphenyl)methyl][(2-hydroxyphenyl)methy l]amino]-2-hydroxybenzoic acid); MNS (3,4-Methylenedioxy-b-nitrostyrene); PP1 (1-(1,1-Dimethylethyl)-1-(4-methylphenyl)-1H-pyrazolo[3,4-d]pyrimidin-4-amine); PP2 (3-(4-chlorophenyl) 1-(1,1-dimethylethyl)-1H-pyrazolo[3,4-d]pyrimidin-4-amine); KX1-004 (Kinex); KX1-005 (Kinex); KX1-136 (Kinex); KX1-174 (Kinex); KX1-141 (Kinex); KX2-328 (Kinex); KX1-306 (Kinex); KX1-329 (Kinex); KX2-391 (Kinex); KX2-377 (Kinex); ZD4190 (Astra Zeneca; N-(4-bromo-2-fluorophenyl)-6-methoxy-7-(2-(1H-1,2,3-triazol-1-yl)ethoxy)quinazolin-4-amine); AP22408 (Ariad Pharmaceuticals); AP23236 (Ariad Pharmaceuticals); AP23451 (Ariad Pharmaceuticals); AP23464 (Ariad Pharmaceuticals); AZD0530 (Astra Zeneca); AZM475271 (M475271; Astra Zeneca); Dasatinib (N-(2-chloro-6-methylphenyl)-2-(6-(4-(2-hydroxyethyl)-piperazin-1-yl)-2-methylpyrimidin-4-ylamino) thiazole-5-carboxamide); GN963 (trans-4-(6,7-dimethoxyquinoxalin-2ylamino)cyclohexanol sulfate); Bosutinib (4-((2,4-dichloro-5-methoxyphenyl)amino)-6-methoxy-7-(3-(4-methyl-1-piperazinyl)propoxy)-3-quinolinecarbonitrile); Hsp70; Hsp72; BiP (or Grp78); mtHsp70 (or Grp75); Hsp70-1b; Hsp70-1L; Hsp70-2; Hsp70-4; Hsp70-6; Hsp70-7; Hsp70-12a; Hsp70-14; Hsp10; Hsp27; Hsp40; Hsp60; Hsp90; Hsp104; Hsp110; Grp94; TFF1; TFF2; TFF3; ApoA; ApoB; ApoC; ApoD; ApoE, ApoH; an siRNA molecule; or combinations thereof. In some embodiments, the anti-apoptotic agent is AM-111 (Auris).

Also disclosed herein, in certain embodiments, is a method for treating an otic disorder comprising administering a composition disclosed herein at least once every 3, 4, 5, 6, 7, 8, 9, 10, 11, 12, 13, 14, or 15 days; at least once a week, once every two weeks, once every three weeks, once every four weeks, once every five weeks, or once every six weeks; or at least once a month, once every two months, once every three months, once every four months, once every five months, once every six months, once every seven months, once every eight months, once every nine months, once every ten months, once every eleven months, or once every twelve months. In particular embodiments, the controlled-release compositions described herein provide a sustained dose of an anti-apoptotic agent or pro-apoptotic agent to the inner ear between subsequent doses of the controlled-release composition. That is, taking one example only, if new doses of the anti-apoptotic agent or pro-apoptotic agent controlled-release composition are administered via intratympanic injection to the round window membrane every 10 days, then the controlled-release composition provides an effective dose of an anti-apoptotic agent or pro-apoptotic agent to the inner ear (e.g., across the round window membrane) during that 10-day period.

In one aspect, the composition is administered so that the composition is in contact with the crista fenestrae cochleae, the round window membrane or the tympanic cavity. In one aspect the composition is administered by intratympanic injection.

Provided herein is a composition or device for use in the treatment of an otic disease or condition characterized by the apoptosis of a plurality of otic cells, comprising: a therapeutically effective amount of an anti-apoptotic agent having substantially low degradation products; and wherein the delivery device comprises two or more characteristics selected from:
(i) between about 0.1% to about 10% by weight of the anti-apoptotic agent, or pharmaceutically acceptable prodrug or salt thereof;
(ii) between about 14% to about 21% by weight of a polyoxyethylene-polyoxypropylene triblock copolymer of general formula E106 P70 E106;
(iii) sterile water, q.s., buffered to provide a pH between about 5.5 and about 8.0;
(iv) multiparticulate anti-apoptotic agent;
(v) a gelation temperature between about 19° C. to about 42° C.;
(vi) less than about 50 colony forming units (cfu) of microbiological agents per gram of delivery device;
(vii) less than about 5 endotoxin units (EU) per kg of body weight of a subject;
(viii) a mean dissolution time of about 30 hours for the; and
(ix) an apparent viscosity of about 100,000 cP to about 500,000 cP.

In some embodiments, a pharmaceutical composition or device described herein comprises:
(i) between about 0.1% to about 10% by weight of the anti-apoptotic agent, or pharmaceutically acceptable prodrug or salt thereof;
(ii) between about 14% to about 21% by weight of a polyoxyethylene-polyoxypropylene triblock copolymer of general formula E106 P70 E106; and
(iii) multiparticulate anti-apoptotic agent.

In some embodiments, a pharmaceutical composition or device described herein comprises:
(i) between about 0.1% to about 10% by weight of the anti-apoptotic agent, or pharmaceutically acceptable prodrug or salt thereof;
(ii) between about 14% to about 21% by weight of a polyoxyethylene-polyoxypropylene triblock copolymer of general formula E106 P70 E106;
(iii) multiparticulate anti-apoptotic agent; and
(iv) a gelation temperature between about 19° C. to about 42° C.

Provided herein composition or device for use in the treatment of an otic disease or condition characterized by the dysfunction of a plurality of otic cells, comprising: a therapeutically effective amount of an anti-apoptotic agent having substantially low degradation products; and wherein the delivery device comprises two or more characteristics selected from:
(i) between about 0.1% to about 10% by weight of the anti-apoptotic agent, or pharmaceutically acceptable prodrug or salt thereof;
(ii) between about 14% to about 21% by weight of a polyoxyethylene-polyoxypropylene triblock copolymer of general formula E106 P70 E106;
(iii) sterile water, q.s., buffered to provide a pH between about 5.5 and about 8.0;
(iv) multiparticulate pro-apoptotic agent;
(v) a gelation temperature between about 19° C. to about 42° C.;
(vi) less than about 50 colony forming units (cfu) of microbiological agents per gram of delivery device;
(vii) less than about 5 endotoxin units (EU) per kg of body weight of a subject;
(viii) a mean dissolution time of about 30 hours for the; and
(ix) an apparent viscosity of about 100,000 cP to about 500,000 cP.

In some embodiments, a pharmaceutical composition or device described herein comprises:
(i) between about 0.1% to about 10% by weight of the pro-apoptotic agent, or pharmaceutically acceptable prodrug or salt thereof;
(ii) between about 14% to about 21% by weight of a polyoxyethylene-polyoxypropylene triblock copolymer of general formula E106 P70 E106; and
(iii) multiparticulate pro-apoptotic agent.

In some embodiments, a pharmaceutical composition or device described herein comprises:
(i) between about 0.1% to about 10% by weight of the pro-apoptotic agent, or pharmaceutically acceptable prodrug or salt thereof;
(ii) between about 14% to about 21% by weight of a polyoxyethylene-polyoxypropylene triblock copolymer of general formula E106 P70 E106;
(iii) multiparticulate pro-apoptotic agent; and
(iv) a gelation temperature between about 19° C. to about 42° C.

In some embodiments, a pharmaceutical composition or device described above provides a practical osmolarity between about 150 and 500 mOsm/L. In some embodiments, a pharmaceutical composition or device described above provides a practical osmolarity between about 200 and 400 mOsm/L. In some embodiments, a pharmaceutical composition or device described above provides a practical osmolarity between about 250 and 320 mOsm/L.

In some embodiments, the anti-apoptotic agent or pro-apoptotic agent is released from the pharmaceutical composition or device described above for a period of at least 3 days. In some embodiments, the anti-apoptotic agent or pro-apoptotic agent is released from the pharmaceutical composition or device described above for a period of at least 5 days. In some embodiments, the anti-apoptotic agent or pro-apoptotic agent is released from the pharmaceutical composition or device described above for a period of at least 10 days. In some embodiments, the anti-apoptotic agent or pro-apoptotic agent is released from the pharmaceutical composition or device described above for a period of at least 14 days. In some embodiments, the anti-apoptotic agent or pro-apoptotic agent is released from the pharmaceutical composition or device described above for a period of at least one month.

In some embodiments, a pharmaceutical composition or device described above comprises an anti-apoptotic agent or pro-apoptotic agent as a neutral molecule, a free acid, a free base, a salt or a prodrug. In some embodiments, a pharmaceutical composition or device described above comprises an anti-apoptotic agent or pro-apoptotic agent as a neutral molecule, a free acid, a free base, a salt or a prodrug, or a combination thereof.

In some embodiments, a pharmaceutical composition or device described above comprises an anti-apoptotic agent or pro-apoptotic agent as multiparticulates. In some embodiments, a pharmaceutical composition or device described above comprises an anti-apoptotic agent or pro-apoptotic agent in the form of micronized particles. In some embodiments, a pharmaceutical composition or device described above comprises an anti-apoptotic agent or pro-apoptotic agent as micronized powders.

In some embodiments, a pharmaceutical composition or device described above comprises about 10% of a polyoxyethylene-polyoxypropylene triblock copolymer of general formula E 106 P70 E106 by weight of the composition. In some embodiments, a pharmaceutical composition or device described above comprises about 15% of a polyoxyethylene-polyoxypropylene triblock copolymer of general formula E 106 P70 E106 by weight of the composition. In some embodiments, a pharmaceutical composition or device described above comprises about 20% of a polyoxyethylene-polyoxypropylene triblock copolymer of general formula E 106 P70 E106 by weight of the composition. In some embodiments, a pharmaceutical composition or device described above comprises about 25% of a polyoxyethylene-polyoxypropylene triblock copolymer of general formula E 106 P70 E106 by weight of the composition.

In some embodiments, a pharmaceutical composition or device described herein comprises about 1% of an anti-apoptotic agent or pro-apoptotic agent, or pharmaceutically acceptable prodrug or salt thereof, by weight of the composition. In some embodiments, a pharmaceutical composition or device described above comprises about 2% of an anti-apoptotic agent or pro-apoptotic agent, or pharmaceutically acceptable prodrug or salt thereof, by weight of the composition. In some embodiments, a pharmaceutical composition or device described herein comprises about 3% of an anti-apoptotic agent or pro-apoptotic agent, or pharmaceutically acceptable prodrug or salt thereof, by weight of the composition. In some embodiments, a pharmaceutical composition or device described herein comprises about 4% of an anti-apoptotic agent or pro-apoptotic agent, or pharmaceutically acceptable prodrug or salt thereof, by weight of the composition. In some embodiments, a pharmaceutical composition or device described above comprises about 5% of an anti-apoptotic agent or pro-apoptotic agent, or pharmaceutically acceptable prodrug or salt thereof, by weight of the composition. In some embodiments, a pharmaceutical composition or device described above comprises about 10% of an anti-apoptotic agent or pro-apoptotic agent, or pharmaceutically acceptable prodrug or salt thereof, by weight of the composition. In some embodiments, a pharmaceutical composition or device described above comprises about 15% of an anti-apoptotic agent or pro-apoptotic agent, or pharmaceutically acceptable prodrug or salt thereof, by weight of the composition. In some embodiments, a pharmaceutical composition or device described above comprises about 20% of an anti-apoptotic agent or pro-apoptotic agent, or pharmaceutically acceptable prodrug or salt thereof, by weight of the composition. In some embodiments, a pharmaceutical composition or device described above comprises about 25% of an anti-apoptotic agent or pro-apoptotic agent, or pharmaceutically acceptable prodrug or salt thereof, by weight of the composition. In some embodiments, a pharmaceutical composition or device described above comprises about 30% of an anti-apoptotic agent or pro-apoptotic agent, or pharmaceutically acceptable prodrug or salt thereof, by weight of the composition. In some embodiments, a pharmaceutical composition or device described above comprises about 40% of an anti-apoptotic agent or pro-apoptotic agent, or pharmaceutically acceptable prodrug or salt thereof, by weight of the composition. In some embodiments, a pharmaceutical composition or device described above comprises about 50% of an anti-apoptotic agent or pro-apoptotic agent, or pharmaceutically acceptable prodrug or salt thereof, by weight of the composition. In some embodiments, a pharmaceutical composition or device described above comprises about 60% of an anti-apoptotic agent or pro-apoptotic agent, or pharmaceutically acceptable prodrug or salt thereof, by weight of the composition. In some embodiments, a pharmaceutical composition or device described above comprises about 70% of an anti-apoptotic agent or pro-apoptotic agent, or pharmaceutically acceptable prodrug or salt thereof, by weight of the composition. In some embodiments, a pharmaceutical composition or device described above comprises about 80% of an anti-apoptotic agent or pro-apoptotic agent, or pharmaceutically acceptable prodrug or salt thereof, by weight of the composition. In some embodiments, a pharmaceutical composition or device described above comprises about 90% of an anti-apoptotic agent or pro-apoptotic agent, or pharmaceutically acceptable prodrug or salt thereof, by weight of the composition.

In some embodiments, a pharmaceutical composition or device described above has a pH between about 5.5 and about 8.0. In some embodiments, a pharmaceutical composition or device described above has a pH between about 6.0 and about 8.0. In some embodiments, a pharmaceutical composition or device described above has a pH between about 6.0 and about 7.6.

In some embodiments, a pharmaceutical composition or device described above contains less than 100 colony forming units (cfu) of microbiological agents per gram of composition. In some embodiments, a pharmaceutical composition or device described above contains less than 50 colony forming units (cfu) of microbiological agents per gram of composition. In some embodiments, a pharmaceutical composition or device described above contains less than 10 colony forming units (cfu) of microbiological agents per gram of composition.

In some embodiments, a pharmaceutical composition or device described above contains less than 5 endotoxin units (EU) per kg of body weight of a subject. In some embodiments, a pharmaceutical composition or device described above contains less than 4 endotoxin units (EU) per kg of body weight of a subject.

In some embodiments, a pharmaceutical composition or device described above provides a gelation temperature between about between about 19° C. to about 42° C. In some embodiments, a pharmaceutical composition or device described above provides a gelation temperature between about between about 19° C. to about 37° C. In some embodiments, a pharmaceutical composition or device described above provides a gelation temperature between about between about 19° C. to about 30° C.

In some embodiments, the pharmaceutical composition or device is an auris-acceptable thermoreversible gel. In some embodiments, the polyoxyethylene-polyoxypropylene triblock copolymer is biodegradable and/or bioeliminated (e.g., the copolymer is eliminated from the body by a biodegradation process, e.g., elimination in the urine, the feces or the like). In some embodiments, a pharmaceutical composition or device described herein further comprises a mucoadhesive. In some embodiments, a pharmaceutical composition or device described herein further comprises a penetration enhancer. In some embodiments, a pharmaceutical composition or device described herein further comprises a thickening agent. In some embodiments, a pharmaceutical composition or device described herein further comprises a dye.

In some embodiments, a pharmaceutical composition or device described herein further comprises a drug delivery device selected from a needle and syringe, a pump, a microinjection device, a wick, an in situ forming spongy material or combinations thereof.

In some embodiments, a pharmaceutical composition or device described herein is a pharmaceutical composition or device wherein the anti-apoptotic agent or pro-apoptotic agent, or pharmaceutically acceptable salt thereof, has limited or no systemic release, systemic toxicity, poor PK characteristics, or combinations thereof. In some embodiments, of the pharmaceutical compositions or devices described herein, the anti-apoptotic agent or pro-apoptotic agent is in the form of a neutral molecule, a free base, a free acid, a salt, a prodrug, or a combination thereof. In some embodiments, of the pharmaceutical compositions or devices described herein, the anti-apoptotic agent or pro-apoptotic agent is administered in the form of a phosphate or ester prodrug. In some embodiments, pharmaceutical compositions or devices described herein comprise one or more anti-apoptotic agent or pro-apoptotic agent, or pharmaceutically acceptable salt thereof, prodrug or combination thereof as an immediate release agent.

In some embodiments, pharmaceutical compositions or devices described herein further comprise an additional therapeutic agent. In some embodiments, the additional therapeutic agent is a an acidifying agent, an anesthetic, an analgesic, an antibiotic, antiemetic, an antifungal, an anti-microbial agent, an antipsychotic (especially those in the phenothiazine class), an antiseptic, an antiviral, an astringent, a chemotherapeutic agent, a collagen, a corticosteroid, a diuretic, a keratolytic agent, a nitric oxide synthase inhibitor, combinations thereof.

In some embodiments, pharmaceutical compositions or devices described herein are pharmaceutical compositions or devices wherein the pH of the pharmaceutical composition or device is between about 6.0 to about 7.6.

In some embodiments, of the pharmaceutical compositions or devices described herein, the ratio of a polyoxyethylene-polyoxypropylene triblock copolymer of general formula E 106 P70 E106 to a thickening agent is from about 40:1 to about 5:1. In some embodiments, the thickening agent is carboxymethyl cellulose, hydroxypropyl cellulose or hydroxypropyl methylcellulose.

In some embodiments, the otic disease or condition is excitotoxicity, ototoxicity, presbycusis or combinations thereof.

Also provided herein is a method of treating an otic disease or condition comprising administering to an individual in need thereof an intratympanic composition or device comprising a therapeutically effective amount of an anti-apoptotic agent or pro-apoptotic agent, the composition or device comprising substantially low degradation products of an anti-apoptotic agent or pro-apoptotic agent, the composition or device further comprising two or more characteristics selected from:
(i) between about 0.1% to about 10% by weight of the anti-apoptotic agent or pro-apoptotic agent, or pharmaceutically acceptable prodrug or salt thereof;
(ii) between about 14% to about 21% by weight of a polyoxyethylene-polyoxypropylene triblock copolymer of general formula E106 P70 E106;
(iii) sterile water, q.s., buffered to provide a pH between about 5.5 and about 8.0;
(iv) multiparticulate anti-apoptotic agent or pro-apoptotic agent;
(v) a gelation temperature between about 19° C. to about 42° C.;
(vi) less than about 50 colony forming units (cfu) of microbiological agents per gram of composition, and
(vii) less than about 5 endotoxin units (EU) per kg of body weight of a subject.

In some embodiments of the methods described herein, the anti-apoptotic agent or pro-apoptotic agent is released from the composition or devices for a period of at least 3 days. In some embodiments of the methods described herein, the anti-apoptotic agent or pro-apoptotic agent is released from the composition or device for a period of at least 4 days. In some embodiments of the methods described herein, the anti-apoptotic agent or pro-apoptotic agent is released from the composition or device for a period of at least 5 days. In some embodiments of the methods described herein, the anti-apoptotic agent or pro-apoptotic agent is released from the composition or device for a period of at least 6 days. In some embodiments of the methods described herein, the anti-apoptotic agent or pro-apoptotic agent is released from the composition or device for a period of at least 7 days. In some embodiments of the methods described herein, the anti-apoptotic agent or pro-apoptotic agent is released from the composition or device for a period of at least 8 days. In some embodiments of the methods described herein, the anti-apoptotic agent or pro-apoptotic agent is released from the composition or device for a period of at least 9 days. In some embodiments of the methods described herein, the anti-apoptotic agent or pro-apoptotic agent is released from the composition or device for a period of at least 10 days. In some embodiments of the method described above, the anti-apoptotic agent or pro-apoptotic agent is essentially in the form of micronized particles.

In some embodiments of the methods described herein, the composition is administered across the round window. In some embodiments of the methods described herein, the otic disease or condition is excitotoxicity, ototoxicity, presbycusis or combinations thereof.

### BRIEF DESCRIPTION OF FIGURES

FIG. 1 illustrates a comparison of non-sustained release and sustained release compositions.

FIG. 2 illustrates the effect of concentration on the viscosity of aqueous solutions of Blanose refined CMC.

FIG. 3 illustrates the effect of concentration on the viscosity of aqueous solutions of Methocel.

FIG. 4 provides an illustrative representation of the anatomy of the ear.

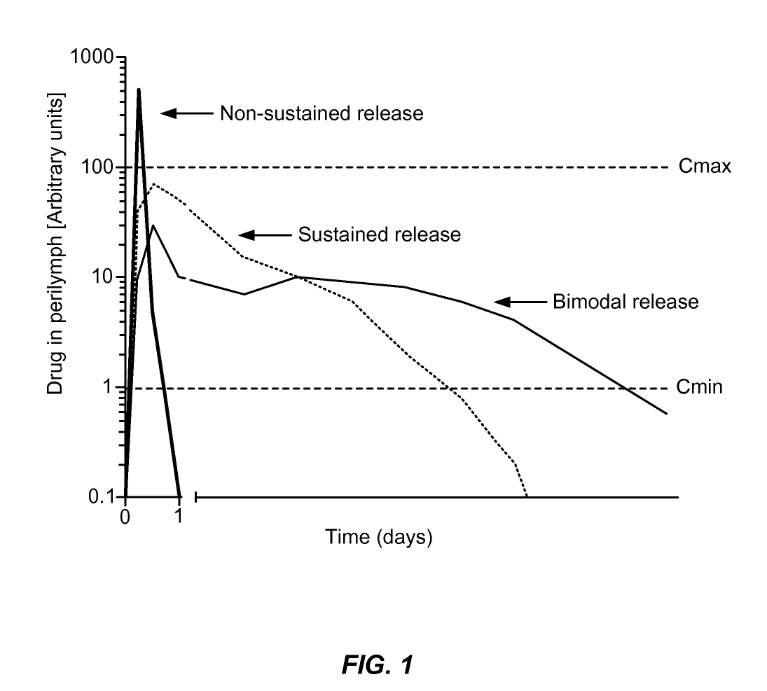

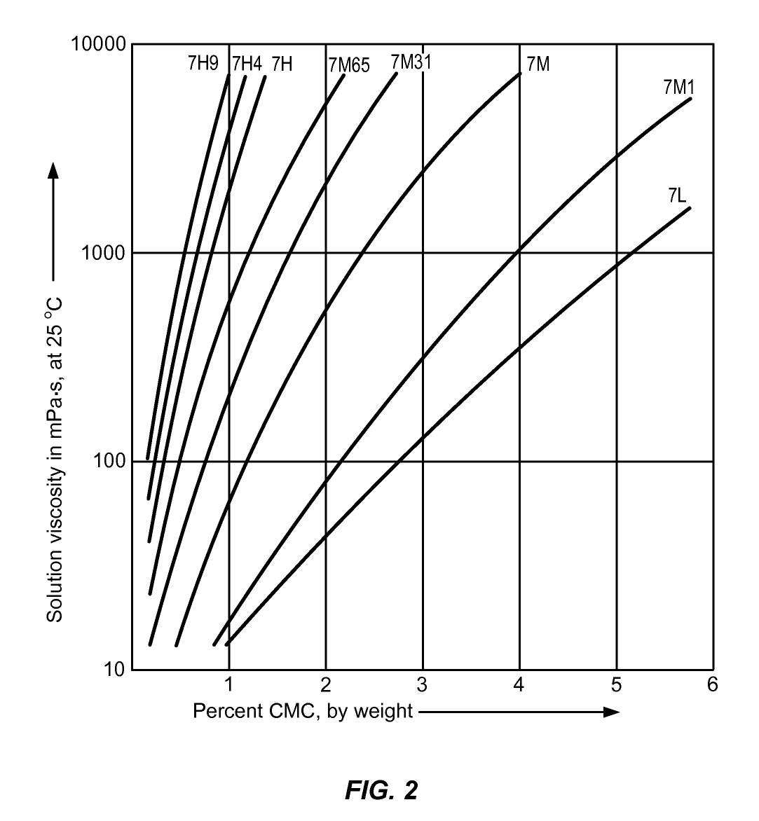

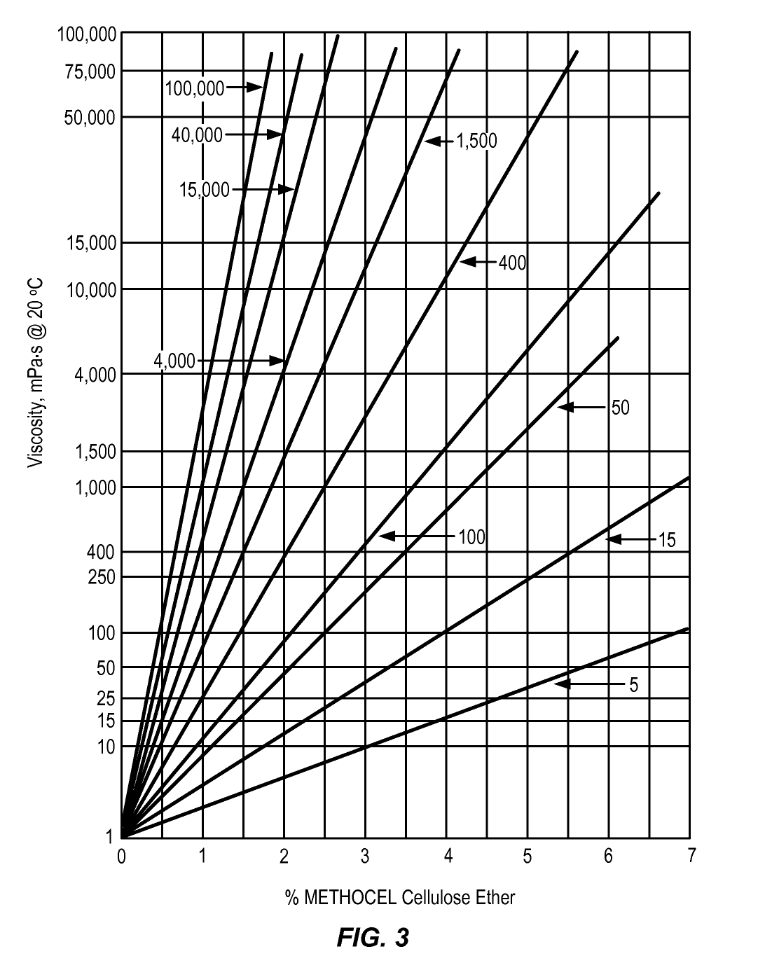

### DETAILED DESCRIPTION OF THE INVENTION

Provided herein are controlled-release anti-apoptotic agent or pro-apoptotic agent compositions and compositions to treat (e.g., ameliorate or reduce the effects of) excitotoxicity, ototoxicity, presbycusis or combinations thereof.

A few therapeutic products are available for the treatment of otic disorders; however, systemic routes via oral, intravenous or intramuscular routes are currently used to deliver these therapeutic agents. In some instances, systemic drug administration creates a potential inequality in drug concentration with higher circulating levels in the serum, and lower levels in the target auris media and auris interna organ structures. As a result, fairly large amounts of drug are required to overcome this inequality in order to deliver sufficient, therapeutically effective quantities to the inner ear. In addition, systemic drug administration may increase the likelihood of systemic toxicities and adverse side effects as a result of the high serum amounts required to effectuate sufficient local delivery to the target site. Systemic toxicities may also occur as a result of liver breakdown and processing of the therapeutic agents, forming toxic metabolites that effectively erase any benefit attained from the administered therapeutic.

To overcome the toxic and attendant side effects of systemic delivery, disclosed herein are methods and compositions and devices for local delivery of therapeutic agents to targeted auris structures. Access to, for example, the vestibular and cochlear apparatus will occur through the auris media including round window membrane, the oval window/stapes footplate, the annular ligament and through the otic capsule/temporal bone.

Intratympanic injection of therapeutic agents is the technique of injecting a therapeutic agent behind the tympanic membrane into the auris media and/or auris interna. This technique presents several challenges; for example, access to the round window membrane, the site of drug absorption into the auris interna, is challenging.

Further, intra-tympanic injections create several unrecognized problems not addressed by currently available treatment regimens, such as changing the osmolarity and pH of the perilymph and endolymph, and introducing pathogens and endotoxins that directly or indirectly damage inner ear structures. One of the reasons the art may not have recognized these problems is that there are no approved intra-tympanic compositions: the inner ear provides sui generis composition challenges. Thus, compositions developed for other parts of the body have little to no relevance for an intra-tympanic composition.

There is no guidance in the prior art regarding requirements (e.g., level of sterility, pH, osmolarity) for otic compositions that are suitable for administration to humans. There is wide anatomical disparity between the ears of animals across species. A consequence of the inter-species differences in auditory structures is that animal models of inner ear disease are often unreliable as a tool for testing therapeutics that are being developed for clinical approval.

Provided herein are otic compositions that meet stringent criteria for pH, osmolarity, ionic balance, sterility, endotoxin and/or pyrogen levels. The auris compositions described herein are compatible with the microenvironment of the inner ear (e.g., the perilymph) and are suitable for administration to humans. In some embodiments, the compositions described herein comprise dyes and aid visualization of the administered compositions obviating the need for invasive procedures (e.g., removal of perilymph) during preclinical and/or clinical development of intratympanic therapeutics.

Provided herein are controlled-release anti-apoptotic agent or pro-apoptotic agent compositions and compositions to locally treat targeted auris structures, thereby avoiding side effects as a result of systemic administration of the anti-apoptotic agent or pro-apoptotic agent compositions and compositions. The locally applied anti-apoptotic agent or pro-apoptotic agent compositions and compositions and devices are compatible with the targeted auris structures, and administered either directly to the desired targeted auris structure (e.g., the cochlear region, the tympanic cavity or the external ear), or administered to a structure in direct communication with areas of the auris interna (e.g., the round window membrane, the crista fenestrae cochleae or the oval window membrane). By specifically targeting an auris structure, adverse side effects as a result of systemic treatment are avoided. Moreover, clinical studies have shown the benefit of having long term exposure of drug to the perilymph of the cochlea, for example with improved clinical efficacy of sudden hearing loss when the therapeutic agent is given on multiple occasions. Thus, by providing a controlled-release apoptosis modulating composition or composition to treat otic disorders, a constant, variable and/or extended source of an anti-apoptotic agent or pro-apoptotic agent is provided to the subject suffering from an otic disorder, reducing or eliminating uncertainty in treatment. Accordingly, one embodiment disclosed herein is to provide a composition that enables at least one anti-apoptotic agent or pro-apoptotic agent to be released in therapeutically effective doses either at variable or constant rates such as to ensure a continuous release of an anti-apoptotic agent or pro-apoptotic agent. In some embodiments, an anti-apoptotic agent or pro-apoptotic agent disclosed herein is administered as an immediate release composition or composition. In other embodiments, an anti-apoptotic agent or pro-apoptotic agent is administered as a sustained release composition, released continuously, variably or in a pulsatile manner, or variants thereof. In still other embodiments, an anti-apoptotic agent or pro-apoptotic agent composition is administered as both an immediate release and sustained release composition, released either continuously, variably or in a pulsatile manner, or variants thereof. The release is optionally dependent on environmental or physiological conditions, for example, the external ionic environment (see, e.g. Oros® release system, Johnson & Johnson).

In addition, localized treatment of the targeted auris structure also affords the use of previously undesired therapeutic agents, including agents with poor pK profiles, poor uptake, low systemic release and/or toxicity issues. Because of the localized targeting of the anti-apoptotic agent or pro-apoptotic agent compositions and compositions and devices, as well as the biological blood barrier present in the auris interna, the risk of adverse effects will be reduced as a result of treatment with previously characterized toxic or ineffective anti-apoptotic agent or pro-apoptotic agents. Accordingly, also contemplated within the scope of the embodiments herein is the use of an anti-apoptotic agent or pro-apoptotic agent in the treatment of disorders that have been previously rejected by practitioners because of adverse effects or ineffectiveness of the anti-apoptotic agent or pro-apoptotic agent.

Also included within the embodiments disclosed herein is the use of additional auris-compatible agents in combination with the anti-apoptotic agent or pro-apoptotic agent compositions and compositions and devices disclosed herein. When used, such agents assist in the treatment of hearing or equilibrium loss or dysfunction as a result of excitotoxicity, ototoxicity, presbycusis or combinations thereof. Accordingly, additional agents that ameliorate or reduce the effects of excitotoxicity, ototoxicity, presbycusis or combinations thereof are also contemplated to be used in combination with an anti-apoptotic agent or pro-apoptotic agent. In some embodiments, the additional agent is an acidifying agent, an anesthetic, an analgesic, an antibiotic, antiemetic, an antifungal, an anti-microbial agent, an antipsychotic (especially those in the phenothiazine class), an antiseptic, an antiviral, an astringent, a chemotherapeutic agent, a collagen, a corticosteroid, a diuretic, a keratolytic agent, a nitric oxide synthase inhibitor, or combinations thereof.

In some embodiments, an auris-acceptable controlled-release apoptosis modulating composition described herein is administered to the target ear region and an oral dose of an anti-apoptotic agent or pro-apoptotic agent is additionally administered. In some embodiments, an oral dose of an anti-apoptotic agent or pro-apoptotic agent is administered before administration of the auris-acceptable controlled-release apoptosis modulating composition, and then the oral dose is tapered off over the period of time that the controlled-release apoptosis modulating composition is provided. Alternatively, an oral dose of an anti-apoptotic agent or pro-apoptotic agent is administered during administration of the controlled-release apoptosis modulating composition, and then the oral dose is tapered off over the period of time that the controlled-release apoptosis modulating composition is provided. Alternatively, an oral dose of an anti-apoptotic agent or pro-apoptotic agent is administered after administration of the controlled-release apoptosis modulating composition, and then the oral dose is tapered off over the period of time that the controlled-release apoptosis modulating composition is provided.

In addition, the anti-apoptotic agent or pro-apoptotic agent pharmaceutical compositions or compositions or devices included herein also include carriers, adjuvants (e.g., preserving, stabilizing, wetting or emulsifying agents), solution promoters, salts for regulating the osmotic pressure, and/or buffers. Such carriers, adjuvants, and other excipients will be compatible with the environment in the targeted auris structure(s). Specifically contemplated are carriers, adjuvants and excipients that lack ototoxicity or are minimally ototoxic in order to allow effective treatment of the otic disorders contemplated herein with minimal side effects in the targeted regions or areas. To prevent ototoxicity, anti-apoptotic agent or pro-apoptotic agent pharmaceutical compositions or compositions or devices disclosed herein are optionally targeted to distinct regions of the targeted auris structures, including but not limited to the tympanic cavity, vestibular bony and membranous labyrinths, cochlear bony and membranous labyrinths and other anatomical or physiological structures located within the auris interna.

### CERTAIN DEFINITIONS

The term “auris-acceptable” with respect to a composition, composition or ingredient, as used herein, includes having no persistent detrimental effect on the auris media (or middle ear) and the auris interna (or inner ear) of the subject being treated. By “auris-pharmaceutically acceptable,” as used herein, refers to a material, such as a carrier or diluent, which does not abrogate the biological activity or properties of the compound in reference to the auris media (or middle ear) and the auris interna (or inner ear), and is relatively or is reduced in toxicity to the auris media (or middle ear) and the auris interna (or inner ear), i.e., the material is administered to an individual without causing undesirable biological effects or interacting in a deleterious manner with any of the components of the composition in that it is contained.

As used herein, amelioration or lessening of the symptoms of a particular otic disease, disorder or condition by administration of a particular compound or pharmaceutical composition refers to any decrease of severity, delay in onset, slowing of progression, or shortening of duration, whether permanent or temporary, lasting or transient that is attributed to or associated with administration of the compound or composition.

As used herein, the term “agonist” means a molecule that binds to and positively alters the activity of a receptor. In some embodiments, an agonist increases the rate at which a receptor functions. In some embodiments, an agonist activates a receptor. In some embodiments, an agonist constitutively activates a receptor. In some embodiments, an agonist increases the accessibility of a receptor's ligand binding pocket (e.g., changes the shape of the binding pocket to make it more accessible). Agonists include, but are not limited to, full agonists, partial agonists, and co-agonists.

As used herein, the term “antagonist” means a molecule that binds to and negatively alters the activity of a receptor. In some embodiments, an antagonist inhibits (partially or fully) the activity of a receptor. In some embodiments, an antagonist decreases the rate at which a receptor functions. In some embodiments, an antagonist decreases the accessibility of a receptor's ligand binding pocket (e.g., blocks a receptor's ligand binding pocket or changes the shape of the binding pocket to make it less accessible). Antagonists include, but are not limited to, competitive antagonists, partial agonists (their binding inhibits the binding of a full agonist), inverse agonists, uncompetitive antagonists, allosteric antagonists, and/or orthosteric antagonists.

“Antioxidants” are auris-pharmaceutically acceptable antioxidants, and include, for example, butylated hydroxytoluene (BHT), sodium ascorbate, ascorbic acid, sodium metabisulfite and tocopherol. In certain embodiments, antioxidants enhance chemical stability where required. Antioxidants are also used to counteract the ototoxic effects of certain therapeutic agents, including agents that are used in combination with the anti-apoptotic agent or pro-apoptotic agents disclosed herein.

“Auris interna” refers to the inner ear, including the cochlea and the vestibular labyrinth, and the round window that connects the cochlea with the middle ear.

“Auris-bioavailability” or “Auris-interna bio availability” or “Auris-media bio availability” or “Auris-externa bioavailability” refers to the percentage of the administered dose of compounds disclosed herein that becomes available in the targeted auris structure of the animal or human being studied.

“Auris media” refers to the middle ear, including the tympanic cavity, auditory ossicles and oval window, which connects the middle ear with the inner ear.

“Auris externa” refers to the outer ear, including the pinna, the auditory canal, and the tympanic membrane, which connects the outer ear with the middle ear.

“Blood plasma concentration” refers to the concentration of compounds provided herein in the plasma component of blood of a subject.

“Carrier materials” are excipients that are compatible with anti-apoptotic agent or pro-apoptotic agent(s), the targeted auris structure(s) and the release profile properties of the auris-acceptable pharmaceutical compositions. Such carrier materials include, e.g., binders, suspending agents, disintegration agents, filling agents, surfactants, solubilizers, stabilizers, lubricants, wetting agents, diluents, and the like. “Auris-pharmaceutically compatible carrier materials” include, but are not limited to, acacia, gelatin, colloidal silicon dioxide, calcium glycerophosphate, calcium lactate, maltodextrin, glycerine, magnesium silicate, polyvinylpyrrolidone (PVP), cholesterol, cholesterol esters, sodium caseinate, soy lecithin, taurocholic acid, phosphatidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like.

“Modulator of apoptosis,” “apoptotic modulating agent” and “anti-apoptotic agent or pro-apoptotic agent” are synonyms. They include agents that (a) protect neurons and otic hair cells (or other cells of the auris media or auris interna) from apoptosis (i.e., anti-apoptotic agents); or agents that induce apoptosis in a neuron or otic hair cell (or other cells of the auris media or auris interna).

The term “diluent” refers to chemical compounds that are used to dilute the anti-apoptotic agent or pro-apoptotic agent prior to delivery and that are compatible with the targeted auris structure(s).

“Dispersing agents,” and/or “viscosity modulating agents” are materials that control the diffusion and homogeneity of the anti-apoptotic agent or pro-apoptotic agent through liquid media. Examples of diffusion facilitators/dispersing agents include but are not limited to hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronic F127, Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol has a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans or combinations thereof. Plasticizers such as cellulose or triethyl cellulose are also be used as dispersing agents. Optional dispersing agents useful in liposomal dispersions and self-emulsifying dispersions of the anti-apoptotic agent or pro-apoptotic agents disclosed herein are dimyristoyl phosphatidyl choline, phosphatidyl cholines (c8-c18), phosphatidylethanolamines (c8-c18), phosphatidyl glycerols (c8-c18), natural phosphatidyl choline from eggs or soy, natural phosphatidyl glycerol from eggs or soy, cholesterol and isopropyl myristate.

“Drug absorption” or “absorption” refers to the process of movement of the anti-apoptotic agent or pro-apoptotic agent(s) from the localized site of administration, by way of example only, the round window membrane of the inner ear, and across a barrier (the round window membranes, as described below) into the auris interna or inner ear structures. The terms “co-administration” or the like, as used herein, are meant to encompass administration of the anti-apoptotic agent or pro-apoptotic agents to a single patient, and are intended to include treatment regimens in that the anti-apoptotic agent or pro-apoptotic agents are administered by the same or different route of administration or at the same or different time.

The terms “effective amount” or “therapeutically effective amount,” as used herein, refer to a sufficient amount of the anti-apoptotic agent or pro-apoptotic agents being administered that would be expected to relieve to some extent one or more of the symptoms of the disease or condition being treated. For example, the result of administration of the anti-apoptotic agent or pro-apoptotic agents disclosed herein is reduction and/or alleviation of the signs, symptoms, or causes of excitotoxicity. For example, an “effective amount” for therapeutic uses is the amount of the anti-apoptotic agent or pro-apoptotic agent, including a composition as disclosed herein required to provide a decrease or amelioration in disease symptoms without undue adverse side effects. The term “therapeutically effective amount” includes, for example, a prophylactically effective amount. An “effective amount” of an anti-apoptotic agent or pro-apoptotic agent composition disclosed herein is an amount effective to achieve a desired pharmacologic effect or therapeutic improvement without undue adverse side effects. It is understood that “an effective amount” or “a therapeutically effective amount” varies, in some embodiments, from subject to subject, due to variation in metabolism of the compound administered, age, weight, general condition of the subject, the condition being treated, the severity of the condition being treated, and the judgment of the prescribing physician. It is also understood that “an effective amount” in an extended-release dosing format may differ from “an effective amount” in an immediate-release dosing format based upon pharmacokinetic and pharmacodynamic considerations.

The terms “enhance” or “enhancing” refers to an increase or prolongation of either the potency or duration of a desired effect of the anti-apoptotic agent or pro-apoptotic agent, or a diminution of any adverse symptoms such as localized pain that is consequent upon administration of the therapeutic agent. Thus, in regard to enhancing the effect of the anti-apoptotic agent or pro-apoptotic agents disclosed herein, the term “enhancing” refers to the ability to increase or prolong, either in potency or duration, the effect of other therapeutic agents that are used in combination with the anti-apoptotic agent or pro-apoptotic agents disclosed herein. An “enhancing-effective amount,” as used herein, refers to an amount of an anti-apoptotic agent or pro-apoptotic agent, or other therapeutic agent, which is adequate to enhance the effect of another therapeutic agent or anti-apoptotic agent or pro-apoptotic agents in a desired system. When used in a patient, amounts effective for this use will depend on the severity and course of the disease, disorder or condition, previous therapy, the patient's health status and response to the drugs, and the judgment of the treating physician.

The term “inhibiting” includes preventing, slowing, or reversing the development of a condition, for example, excitotoxicity, or advancement of a condition in a patient necessitating treatment.

The terms “kit” and “article of manufacture” are used as synonyms.

“Pharmacokinetics” refers to the factors that determine the attainment and maintenance of the appropriate concentration of drug at the desired site within the targeted auris structure.

In prophylactic applications, compositions containing an anti-apoptotic agent or pro-apoptotic agent described herein are administered to a patient susceptible to or otherwise at risk of a particular disease, disorder or condition, for example, excitotoxicity, ototoxicity, and presbycusis. Such an amount is defined to be a “prophylactically effective amount or dose.” In this use, the precise amounts also depend on the patient's state of health, weight, and the like. As used herein, a “pharmaceutical device” includes any composition described herein that, upon administration to an ear, provides a reservoir for extended release of an active agent described herein.

A “prodrug” refers to the anti-apoptotic agent or pro-apoptotic agent that is converted into the parent drug in vivo. In certain embodiments, a prodrug is enzymatically metabolized by one or more steps or processes to the biologically, pharmaceutically or therapeutically active form of the compound. To produce a prodrug, a pharmaceutically active compound is modified such that the active compound will be regenerated upon in vivo administration. In one embodiment, the prodrug is designed to alter the metabolic stability or the transport characteristics of a drug, to mask side effects or toxicity, or to alter other characteristics or properties of a drug. Compounds provided herein, in some embodiments, are derivatized into suitable prodrugs.

“Round window membrane” is the membrane in humans that covers the fenestrae cochlea (also known as the circular window, fenestrae rotunda, or round window). In humans, the thickness of round window membrane is about 70 micron.

“Solubilizers” refers to auris-acceptable compounds such as triacetin, triethylcitrate, ethyl oleate, ethyl caprylate, sodium lauryl sulfate, sodium caprate, sucrose esters, alkylglucosides, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” refers to compounds such as any antioxidation agents, buffers, acids, preservatives and the like that are compatible with the environment of the targeted auris structure. Stabilizers include but are not limited to agents that will do any of (1) improve the compatibility of excipients with a container, or a delivery system, including a syringe or a glass bottle, (2) improve the stability of a component of the composition, or (3) improve composition stability.

“Steady state,” as used herein, is when the amount of drug administered to the targeted auris structure is equal to the amount of drug eliminated within one dosing interval resulting in a plateau or constant levels of drug exposure within the targeted structure.

As used herein, the term “substantially low degradation products” means less than 5% by weight of the active agent are degradation products of the active agent. In further embodiments, the term means less than 3% by weight of the active agent are degradation products of the active agent. In yet further embodiments, the term means less than 2% by weight of the active agent are degradation products of the active agent. In further embodiments, the term means less than 1% by weight of the active agent are degradation products of the active agent.

As used herein, the term “subject” is used to mean any animal, preferably a mammal, including a human or non-human. The terms patient and subject may be used interchangeably. Neither term is to be interpreted as requiring the supervision of a medical professional (e.g., a doctor, nurse, physician's assistant, orderly, hospice worker).

“Surfactants” refers to compounds that are auris-acceptable, such as sodium lauryl sulfate, sodium docusate, Tween® 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants are included to enhance physical stability or for other purposes.

The terms “treat,” “treating” or “treatment,” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

As used herein “essentially in the form of micronized powder” includes, by way of example only, greater than 70% by weight of the active agent is in the form of micronized particles of the active agent. In further embodiments, the term means greater than 80% by weight of the active agent is in the form of micronized particles of the active agent. In yet further embodiments, the term means greater than 90% by weight of the active agent is in the form of micronized particles of the active agent.

As used herein, the term “substantially low degradation products” means less than 5% by weight of the active agent are degradation products of the active agent. In further embodiments, the term means less than 3% by weight of the active agent are degradation products of the active agent. In yet further embodiments, the term means less than 2% by weight of the active agent are degradation products of the active agent. In further embodiments, the term means less than 1% by weight of the active agent are degradation products of the active agent.

As used herein, the term “otic intervention” means an external insult or trauma to one or more auris structures and includes implants, otic surgery, injections, cannulations, or the like. Implants include auris-interna or auris-media medical devices, examples of which include cochlear implants, hearing sparing devices, hearing-improvement devices, short electrodes, micro-prostheses or piston-like prostheses; needles; stem cell transplants; drug delivery devices; a cell-based therapeutic; or the like. Otic surgery includes middle ear surgery, inner ear surgery, typanostomy, cochleostomy, labyrinthotomy, mastoidectomy, stapedectomy, stapedotomy, endolymphatic sacculotomy or the like. Injections include intratympanic injections, intracochlear injections, injections across the round window membrane or the like. Cannulations include intratympanic, intracochlear, endolymphatic, perilymphatic or vestibular cannulations or the like.

Other objects, features, and advantages of the methods and compositions described herein will become apparent from the following detailed description. It should be understood, however, which the detailed description and the specific examples, while indicating specific embodiments, are given by way of illustration only.

### Anatomy of the Ear

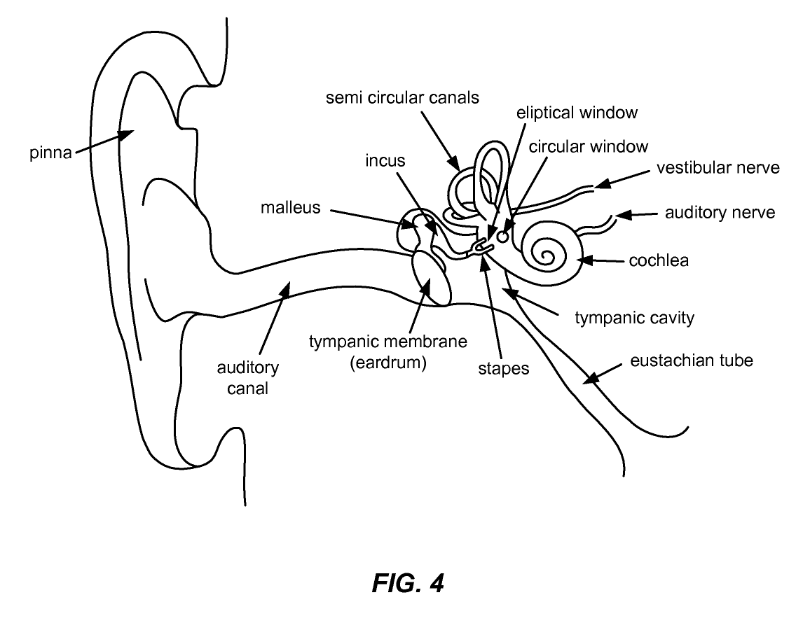

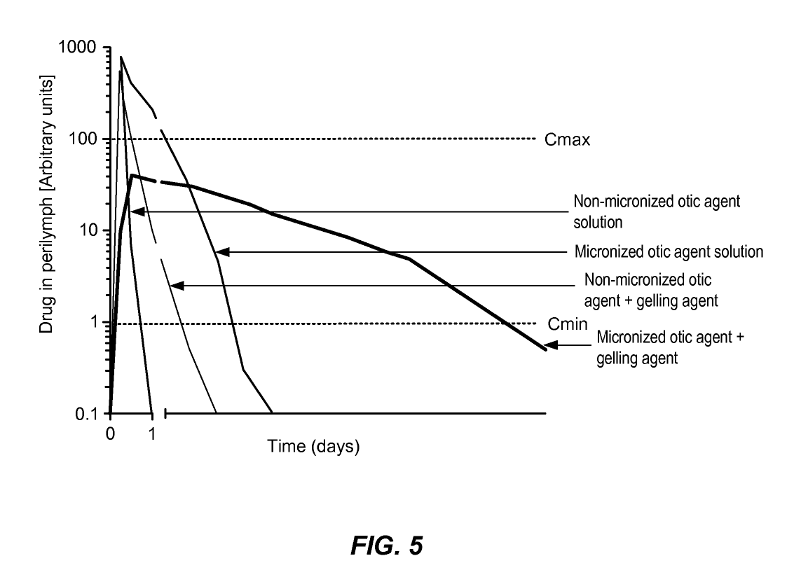

As shown in FIG. 4, the outer ear is the external portion of the organ and is composed of the pinna (auricle), the auditory canal (external auditory meatus) and the outward facing portion of the tympanic membrane, also known as the ear drum. The pinna, which is the fleshy part of the external ear that is visible on the side of the head, collects sound waves and directs them toward the auditory canal. Thus, the function of the outer ear, in part, is to collect and direct sound waves towards the tympanic membrane and the middle ear.

The middle ear is an air-filled cavity, called the tympanic cavity, behind the tympanic membrane. The tympanic membrane, also known as the ear drum, is a thin membrane that separates the external ear from the middle ear. The middle ear lies within the temporal bone, and includes within this space the three ear bones (auditory ossicles): the malleus, the incus and the stapes. The auditory ossicles are linked together via tiny ligaments, which form a bridge across the space of the tympanic cavity. The malleus, which is attached to the tympanic membrane at one end, is linked to the incus at its anterior end, which in turn is linked to the stapes. The stapes is attached to the oval window, one of two windows located within the tympanic cavity. A fibrous tissue layer, known as the annular ligament connects the stapes to the oval window. Sound waves from the outer ear first cause the tympanic membrane to vibrate. The vibration is transmitted across to the cochlea through the auditory ossicles and oval window, which transfers the motion to the fluids in the auris interna. Thus, the auditory ossicles are arranged to provide a mechanical linkage between the tympanic membrane and the oval window of the fluid-filled auris interna, where sound is transformed and transduced to the auris interna for further processing. Stiffness, rigidity or loss of movement of the auditory ossicles, tympanic membrane or oval window leads to hearing loss, e.g. otosclerosis, or rigidity of the stapes bone.

The tympanic cavity also connects to the throat via the eustachian tube. The eustachian tube provides the ability to equalize the pressure between the outside air and the middle ear cavity. The round window, a component of the auris interna but that is also accessible within the tympanic cavity, opens into the cochlea of the auris interna. The round window is covered by round window membrane, which consists of three layers: an external or mucous layer, an intermediate or fibrous layer, and an internal membrane, which communicates directly with the cochlear fluid. The round window, therefore, has direct communication with the auris interna via the internal membrane.

Movements in the oval and round window are interconnected, i.e. as the stapes bone transmits movement from the tympanic membrane to the oval window to move inward against the auris interna fluid, the round window (round window membrane) is correspondingly pushed out and away from the cochlear fluid. This movement of the round window allows movement of fluid within the cochlea, which leads in turn to movement of the cochlear inner hair cells, allowing hearing signals to be transduced. Stiffness and rigidity in round window membrane leads to hearing loss because of the lack of ability of movement in the cochlear fluid. Recent studies have focused on implanting mechanical transducers onto the round window, which bypasses the normal conductive pathway through the oval window and provides amplified input into the cochlear chamber.

Auditory signal transduction takes place in the auris interna. The fluid-filled auris interna, or inner ear, consists of two major components: the cochlear and the vestibular apparatus. The auris interna is located in part within the osseous or bony labyrinth, an intricate series of passages in the temporal bone of the skull. The vestibular apparatus is the organ of balance and consists of the three semi-circular canals and the vestibule. The three semi-circular canals are arranged relative to each other such that movement of the head along the three orthogonal planes in space can be detected by the movement of the fluid and subsequent signal processing by the sensory organs of the semi-circular canals, called the crista ampullaris. The crista ampullaris contains hair cells and supporting cells, and is covered by a dome-shaped gelatinous mass called the cupula. The hairs of the hair cells are embedded in the cupula. The semi-circular canals detect dynamic equilibrium, the equilibrium of rotational or angular movements.

When the head turns rapidly, the semicircular canals move with the head, but endolymph fluid located in the membranous semi-circular canals tends to remain stationary. The endolymph fluid pushes against the cupula, which tilts to one side. As the cupula tilts, it bends some of the hairs on the hair cells of the crista ampullaris, which triggers a sensory impulse. Because each semicircular canal is located in a different plane, the corresponding crista ampullaris of each semi-circular canal responds differently to the same movement of the head. This creates a mosaic of impulses that are transmitted to the central nervous system on the vestibular branch of the vestibulocochlear nerve. The central nervous system interprets this information and initiates the appropriate responses to maintain balance. Of importance in the central nervous system is the cerebellum, which mediates the sense of balance and equilibrium.

The vestibule is the central portion of the auris interna and contains mechanoreceptors bearing hair cells that ascertain static equilibrium, or the position of the head relative to gravity. Static equilibrium plays a role when the head is motionless or moving in a straight line. The membranous labyrinth in the vestibule is divided into two sac-like structures, the utricle and the saccule. Each structure in turn contains a small structure called a macula, which is responsible for maintenance of static equilibrium. The macula consists of sensory hair cells, which are embedded in a gelatinous mass (similar to the cupula) that covers the macula. Grains of calcium carbonate, called otoliths, are embedded on the surface of the gelatinous layer.

When the head is in an upright position, the hairs are straight along the macula. When the head tilts, the gelatinous mass and otoliths tilts correspondingly, bending some of the hairs on the hair cells of the macula. This bending action initiates a signal impulse to the central nervous system, which travels via the vestibular branch of the vestibulocochlear nerve, which in turn relays motor impulses to the appropriate muscles to maintain balance.

The cochlea is the portion of the auris interna related to hearing. The cochlea is a tapered tube-like structure that is coiled into a shape resembling a snail. The inside of the cochlea is divided into three regions, which is further defined by the position of the vestibular membrane and the basilar membrane. The portion above the vestibular membrane is the scala vestibuli, which extends from the oval window to the apex of the cochlea and contains perilymph fluid, an aqueous liquid low in potassium and high in sodium content. The basilar membrane defines the scala tympani region, which extends from the apex of the cochlea to the round window and also contains perilymph. The basilar membrane contains thousands of stiff fibers, which gradually increase in length from the round window to the apex of the cochlea. The fibers of the basement membrane vibrate when activated by sound. In between the scala vestibuli and the scala tympani is the cochlear duct, which ends as a closed sac at the apex of the cochlea. The cochlear duct contains endolymph fluid, which is similar to cerebrospinal fluid and is high in potassium.

The organ of Corti, the sensory organ for hearing, is located on the basilar membrane and extends upward into the cochlear duct. The organ of Corti contains hair cells, which have hairlike projections that extend from their free surface, and contacts a gelatinous surface called the tectorial membrane. Although hair cells have no axons, they are surrounded by sensory nerve fibers that form the cochlear branch of the vestibulocochlear nerve (cranial nerve VIII).

As discussed, the oval window, also known as the elliptical window communicates with the stapes to relay sound waves that vibrate from the tympanic membrane. Vibrations transferred to the oval window increase pressure inside the fluid-filled cochlea via the perilymph and scala vestibuli/scala tympani, which in turn cause the round window membrane to expand in response. The concerted inward pressing of the oval window/outward expansion of the round window allows for the movement of fluid within the cochlea without a change of intra-cochlear pressure. However, as vibrations travel through the perilymph in the scala vestibuli, they create corresponding oscillations in the vestibular membrane. These corresponding oscillations travel through the endolymph of the cochlear duct, and transfer to the basilar membrane. When the basilar membrane oscillates, or moves up and down, the organ of Corti moves along with it. The hair cell receptors in the Organ of Corti then move against the tectorial membrane, causing a mechanical deformation in the tectorial membrane. This mechanical deformation initiates the nerve impulse that travels via the vestibulocochlear nerve to the central nervous system, mechanically transmitting the sound wave received into signals that are subsequently processed by the central nervous system.

### Diseases

Otic disorders, including auris interna disorders, auris media disorders and auris externa disorders, produce symptoms which include but are not limited to hearing loss, nystagmus, vertigo, tinnitus, inflammation, infection and congestion. The otic disorders which are treated with the compositions disclosed herein are numerous and include ototoxicity, excitotoxicity, and presbycusis.

Excitotoxicity

Excitotoxicity refers to the death of, or damaging of neurons and/or otic hair cells by glutamate and/or similar substances.

Glutamate is the most abundant excitatory neurotransmitter in the central nervous system. Pre-synaptic neurons release glutamate upon stimulation. It flows across the synapse, binds to receptors located on post-synaptic neurons, and activates these neurons. The glutamate receptors include the NMDA, AMPA, and kainate receptors. Glutamate transporters are tasked with removing extracellular glutamate from the synapse. Certain events (e.g. ischemia or stroke) damage the glutamate transporters. This results in excess glutamate accumulating in the synapse. Excess glutamate in synapses results in the over-activation of the glutamate receptors.

The AMPA receptor is activated by the binding of both glutamate and AMPA. Activation of certain isoforms of the AMPA receptor results in the opening of ion channels located in the plasma membrane of the neuron. When the channels open, Na⁺ and Ca²⁺ ions flow into the neuron and K⁺ ions flow out of the neuron.

The NMDA receptor is activated by the binding of both glutamate and NMDA. Activation of the NMDA receptor, results in the opening of ion channels located in the plasma membrane of the neuron. However, these channels are blocked by Mg²⁺ ions. Activation of the AMPA receptor results in the expulsion of Mg²⁺ ions from the ion channels into the synapse. When the ion channels open, and the Mg²⁺ ions evacuate the ion channels, Na⁺ and Ca²⁺ ions flow into the neuron, and K⁺ ions flow out of the neuron.

Excitotoxicity occurs when the NMDA receptor and AMPA receptors are over-activated by the binding of excessive amounts of ligands, for example, abnormal amounts of glutamate. The over-activation of these receptors causes excessive opening of the ion channels under their control. This allows abnormally high levels of Ca²⁺ and Na⁺ to enter the neuron. The influx of these levels of Ca²⁺ and Na⁺ into the neuron causes the neuron to fire more often. This increased firing yields a rapid buildup of free radicals and inflammatory compounds. The free radicals damage the mitochondria, depleting the cell's energy stores. Further, excess levels of Ca²⁺ and Na⁺ ions activate excess levels of enzymes including, but not limited to, phospholipases, endonucleases, and proteases. The over-activation of these enzymes results in damage to the cytoskeleton, plasma membrane, mitochondria, and DNA of the neuron. Such damage often results in the activation of apoptotic genes. Additionally, the transcription of multiple pro-apoptotic genes and apoptosis modulating genes are controlled by Ca²⁺ levels. Excess Ca²⁺ often results in the upregulation of the pro-apoptotic genes and the down-regulation of apoptosis modulating genes.

### Presbycusis

Presbycusis is the progressive bilateral loss of hearing that results from aging. Most hearing loss occurs at higher frequencies (i.e. frequencies above 15 or 16 Hz) making it difficult to hear a female voice (as opposed to male voice), and an inability to differentiate between high-pitched sounds (such as “s” and “th”). It may be difficult filter out background noise. The disorder is most often treated by the implantation of a hearing aid and/or the administration of pharmaceutical agents which prevent the build up of ROS.

The disorder is caused by changes in the physiology of the inner ear, the middle ear, and/or the VII nerve. Changes in the inner ear resulting in presbycusis include epithelial atrophy with loss of otic hair cells and/or stereocilia, atrophy of nerve cells, atrophy of the stria vascularis, and the thickening/stiffening of the basilar membrane. Additional changes which can contribute to presbycusis include the accumulation of defects in the tympanic membrane and the ossicles.

Changes leading to presbycusis can occur due to the accumulation of mutations in DNA, and mutations in mitochondrial DNA; however, the changes may be exacerbated by exposure to loud noise, exposure to ototoxic agents, infections, and/or the lessening of blood flow to the ear. The latter is attributable to atherosclerosis, diabetes, hypertension, and smoking.

### Ototoxicity

Ototoxicity refers to the destruction or damage to a neuron or hair cell of the auris wherein said damage is caused by a toxin. Multiple drugs are known to be ototoxic. Often ototoxicity is dose-dependent. It may be permanent or reversible upon withdrawal of the drug.

Known ototoxic drugs include, but are not limited to, the aminoglycoside class of antibiotics (e.g. gentamicin, and amikacin), some members of the macrolide class of antibiotics (e.g. erythromycin), some members of the glycopeptide class of antibiotics (e.g. vancomycin), salicylic acid, nicotine, some chemotherapeutic agents (e.g. actinomycin, bleomycin, cisplatin, carboplatin and vincristine), and some members of the loop diuretic family of drugs (e.g. furosemide).

Cisplatin, and the aminoglycoside class of antibiotics induce the production of reactive oxygen species (ROS). ROS can damage cells directly, and thus induce apoptosis, by damaging DNA, proteins, and/or lipids. ROS are also involved in the signaling cascade which results in apoptosis. In certain instances, antioxidants prevent damage by ROS by preventing their formation or scavenging free radicals before they can damage the cell. Accordingly, some embodiments incorporate the use of antioxidants. In certain instances, nitrones act synergistically with antioxidants to prevent acute acoustic noise-induced hearing loss. In certain instances, nitrones trap free radicals. In some embodiments, a nitrone (e.g. alpha-phenyl-tert-butylnitrone (PBN), allpurinol) is co-administered with an antioxidant. Both cisplatin and the aminoglycoside class of antibiotics are also thought to damage the ear by binding melanin in the stria vascularis of the inner ear.

Salicylic acid is classified as ototoxic as it inhibits the function of the protein prestin. Prestin mediates outer otic hair cell motility by controlling the exchange of chloride and carbonate across the plasma membrane of outer otic hair cells. It is only found in the outer otic hair cells, not the inner otic hair cells.

Otic and/or vestibular disorders, including auris interna disorders and auris media disorders, produce symptoms which include but are not limited to hearing loss, nystagmus, vertigo, tinnitus, inflammation, swelling, infection and congestion. These disorders may have many causes, such as infection, injury, inflammation, tumors and adverse response to drugs or other chemical agents.

### Trauma

Trauma refers to physical damage to an otic structure resulting from the application of an outside force. In some embodiments, trauma results from exposure to loud noise (e.g., firecrackers, or loud music). In some embodiments, trauma to an otic structure results from the implantation of a medical device, otic surgery, injections, cannulations, or the like. Implants include auris-interna or auris-media medical devices, examples of which include cochlear implants, hearing sparing devices, hearing-improvement devices, short electrodes, micro-prostheses or piston-like prostheses; needles; stem cell transplants; drug delivery devices; a cell-based therapeutic; or the like. Otic surgery includes middle ear surgery, inner ear surgery, typanostomy, cochleostomy, labyrinthotomy, mastoidectomy, stapedectomy, stapedotomy, endolymphatic sacculotomy or the like. Injections include intratympanic injections, intracochlear injections, injections across the round window membrane or the like. Cannulations include intratympanic, intracochlear, endolymphatic, perilymphatic or vestibular cannulations or the like.

In some embodiments, administration of an anti-apoptotic agent (e.g., AM-111) composition or device as described herein in following acoustic trauma delays or prevents damage to auris structures, e.g., irritation, cell death osteoneogeneis and/or further neuronal degeneration, caused by the acoustic trauma.

In some embodiments, administration of an anti-apoptotic agent (e.g., AM-111) composition or device as described herein in combination with the implantation of an exogenous material (e.g., a medical device implant or a stem cell transplant) delays or prevents damage to auris structures, e.g., irritation, cell death osteoneogeneis and/or further neuronal degeneration, caused by installation of an external device and/or a plurality cells (e.g., stem cells) in the ear. In some embodiments, administration of anti-apoptotic agent (e.g., AM-111) composition or device as described herein in combination with an implant allows for a more effective restoration of hearing loss compared to an implant alone.

In some embodiments, administration of an anti-apoptotic agent (e.g., AM-111) composition or device as described herein reduces damage to auris structures caused by underlying conditions allowing for successful implantation. In some embodiments, administration of an anti-apoptotic agent (e.g., AM-111) composition or device as described herein, in conjunction surgery and/or with the implantation of an exogenous material reduces or prevents negative side-effects (e.g., cell death).

In some embodiments, administration of an anti-apoptotic agent (e.g., AM-111) composition or device as described herein in conjunction with the implantation of an exogenous material has a trophic effect (i.e., promotes healthy growth of cells and healing of tissue in the area of an implant or transplant). In some embodiments, a trophic effect is desirable during otic surgery or during intratympanic injection procedures. In some embodiments, a trophic effect is desirable after installation of a medical device or after a cell (e.g., stem cell) transplant. In some of such embodiments, an anti-apoptotic agent (e.g., AM-111) composition or device as described herein is administered via direct cochlear injection, through a chochleostomy or via deposition on the round window

In some embodiments, administration of an anti-apoptotic agent (e.g., AM-111) composition or device as described herein reduces inflammation and/or infections associated with otic surgery, or implantation of an exogenous material (e.g., a medical device or a plurality of cells (e.g., stem cells)). In some instances, perfusion of a surgical area with an anti-apoptotic agent (e.g., AM-111) composition or device as described herein reduces or eliminates post-surgical and/or post-implantation complications (e.g., inflammation, hair cell damage, neuronal degeneration, osteoneogenesis or the like). In some instances, perfusion of a surgical area with an anti-apoptotic agent (e.g., AM-111) composition or device as described herein reduces post-surgery or post-implantation recuperation time.

In one aspect, the formulations described herein, and modes of administration thereof, are applicable to methods of direct perfusion of the inner ear compartments. Thus, the formulations described herein are useful in combination with surgical procedures including, by way of non-limiting examples, cochleostomy, labyrinthotomy, mastoidectomy, stapedectomy, stapedotomy, endolymphatic sacculotomy or the like. In some embodiments, the inner ear compartments are perfused with an anti-apoptotic agent (e.g., AM-111) composition or device as described herein prior to otic surgery, during otic surgery, after otic surgery, or a combination thereof.

In some embodiments, AM-111 is administered to a subject to repair damage to an otic structure from trauma. In some embodiments, AM-111 is administered prior to otic surgery, during otic surgery, after otic surgery to reduce damage resulting from the surgery.

### Pharmaceutical Agents

Provided herein are apoptosis modulating compositions that protect the neurons and otic hair cells of the auris from apoptosis, and/or ameliorate the degeneration of sensory neurons and/or hair cells of the auris. Further provided herein are apoptosis modulating compositions that promote the growth and/or regeneration of neurons and/or hair cells of the auris. Also provided herein are apoptosis modulating compositions that induce apoptosis of neurons and/or hair cells of the auris. Otic disorders have causes and symptoms that are responsive to the pharmaceutical agents disclosed herein, or other pharmaceutical agents. Anti-apoptotic agent or pro-apoptotic agents which are not disclosed herein but which are useful for the amelioration or eradication of otic and/or vestibular disorders are expressly included and intended within the scope of the embodiments presented.

Moreover, pharmaceutical agents which have been previously shown to be toxic, harmful or non-effective during systemic or localized application in other organ systems, for example through toxic metabolites formed after hepatic processing, toxicity of the drug in particular organs, tissues or systems, through high levels needed to achieve efficacy, through the inability to be released through systemic pathways or through poor pK characteristics, are useful in some embodiments herein. For example, known side effects of minocycline, an anti-apoptotic agent or pro-apoptotic agent, includes diarrhea, headache, vomiting, fever, jaundice, intracranial hypertension and autoimmune disorders, such as lupus. Accordingly, pharmaceutical agents which have limited or no systemic release, systemic toxicity, poor pK characteristics or combinations thereof are contemplated within the scope of the embodiments disclosed herein.

The apoptosis modulating compositions disclosed herein are optionally targeted directly to otic structures where treatment is needed; for example, one embodiment contemplated is the direct application of the anti-apoptotic agent or pro-apoptotic agent formulations disclosed herein onto the round window membrane or the crista fenestrae cochlea of the auris interna, allowing direct access and treatment of the auris interna, or inner ear components. In other embodiments, the anti-apoptotic agent or pro-apoptotic agent formulation disclosed herein is applied directly to the oval window. In yet other embodiments, direct access is obtained through microinjection directly into the auris interna, for example, through cochlear microperfusion. Such embodiments also optionally comprise a drug delivery device, wherein the drug delivery device delivers the anti-apoptotic agent or pro-apoptotic agent formulations through use of a needle and syringe, a pump, a microinjection device or any combination thereof.

Some pharmaceutical agents, either alone or in combination, are ototoxic. For example, some chemotherapeutic agents, including actinomycin, bleomycin, cisplatin, carboplatin and vincristine; and antibiotics, including erythromycin, gentamicin, streptomycin, dihydrostreptomycin, tobramycin, netilmicin, amikacin, neomycin, kanamycin, etiomycin, vancomycin, metronidizole, capreomycin, are mildly to very toxic, and affect the vestibular and cochlear structures differentially. However, in some instances, the combination of an ototoxic drug, for example cisplatin, in combination with an otoprotectant is protective by lessening the ototoxic effects of the drug. Moreover, the localized application of the potentially ototoxic drug also lessens the toxic effects that otherwise occur through systemic application through the use of lower amounts with maintained efficacy, or the use of targeted amounts for a shorter period of time.

Moreover, some pharmaceutical excipients, diluents or carriers are potentially ototoxic. For example, benzalkonium chloride, a common preservative, is ototoxic and therefore potentially harmful if introduced into the vestibular or cochlear structures. In formulating a controlled-release anti-apoptotic agent or pro-apoptotic agent formulation, it is advised to avoid or combine the appropriate excipients, diluents or carriers to lessen or eliminate potential ototoxic components from the formulation, or to decrease the amount of such excipients, diluents or carriers. Optionally, a controlled-release anti-apoptotic agent or pro-apoptotic agent formulation includes otoprotective agents, such as antioxidants, alpha lipoic acid, calcium, fosfomycin or iron chelators, to counteract potential ototoxic effects that may arise from the use of specific therapeutic agents or excipients, diluents or carriers. In some embodiments, a nitrone (e.g. alpha-phenyl-tert-butylnitrone) is co-administered with an antioxidant.

Inhibitors of the MAPK/JNK Signaling Cascade

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

The MAPK/JNK cascade induces apoptosis in cells. Cellular stress (e.g. acoustic trauma, exposure to an ototoxic agent, ect.) activates the mitogen-activated protein kinases (MAPK). Activated MAPKs phosphorylate the Thr and Tyr residues on members of the c-Jun N-terminal kinases (JNKs), thus activating these kinases. The JNKs then phosphorylate c-Jun, a component of the AP-1 transcription factor complex. Activation of AP-1 induces the transcription of several pro-apoptotic members of the Bcl-2 family (e.g. Bax, BAD, Bak and Bok).

In some embodiments, the anti-apoptotic agent is an agent which inhibits (partially or fully) the activity of the MAPK/JNK signaling cascade. In some embodiments, the anti-apoptotic agent is minocycline; SB-203580 (4-(4-Fluorophenyl)-2-(4-methylsulfinyl phenyl)-5-(4-pyridyl) 1H-imidazole); PD 169316 (4-(4-Fluorophenyl)-2-(4-nitrophenyl)-5-(4-pyridyl)-1H-imidazole); SB 202190 (4-(4-Fluorophenyl)-2-(4-hydroxyphenyl)-5-(4-pyridyl) 1H-imidazole); RWJ 67657 (4-[4-(4-fluorophenyl)-1-(3-phenylpropyl)-5-(4-pyridinyl)-1H-imidazol-2-yl]-3-butyn-1-ol); SB 220025 (5-(2-Amino-4-pyrimidinyl)-4-(4-fluorophenyl)-1-(4-piperidinlyl)imidazole); or combinations thereof. Minocycline prevents the apoptosis of otic hair cells following treatment with the ototoxic antibiotic gentamicin by inhibiting the induction of p38 MAPK phosphorylation. In some embodiments, the agent which antagonizes the MAPK/JNK signaling cascade is D-JNKI-1 ((D)-hJIP₁₇₅₋₁₅₇-DPro-DPro-(D)-HIV-TAT₅₇₄₈), AM-111 (Auris), SP600125 (anthra[1,9-cd]pyrazol-6(2H)-one), JNK Inhibitor I ((L)-HIV-TAT₄₈₋₅₇-PP-JBD₂₀), JNK Inhibitor III ((L)-HIV-TAT₄₇₋₅₇-gaba-c-Junδ₃₃₋₅₇), AS601245 (1,3-benzothiazol-2-yl (2-[[2-(3-pyridinyl)ethyl]amino]-4 pyrimidinyl) acetonitrile), JNK Inhibitor VI (H₂N-RPKRPTTLNLF-NH₂), JNK Inhibitor VIII (N-(4-Amino-5-cyano-6-ethoxypyridin-2-yl)-2-(2,5-dimethoxyphenyl)acetamide), JNK Inhibitor IX (N-(3-Cyano-4,5,6,7-tetrahydro-1-benzothien-2-yl)-1-naphthamide), dicumarol (3,3′-Methylenebis(4-hydroxycoumarin)), SC-236 (4-[5-(4-chlorophenyl)-3-(trifluoromethyl)-1H-pyrazol-1-yl]benzene-sulfonamide), CEP-1347 (Cephalon), CEP-11004 (Cephalon); or combinations thereof. In some embodiments, the anti-apoptotic agent is AM-111 (Auris).

JAK (Janus Kinase) Inhibitors

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

In certain instances, JAK kinases phosphorylate and activate downstream proteins involved in type I and type II cytokine receptor signal transduction pathways. In certain instances, the activation of a JAK2 kinase induces apoptosis.

In some embodiments, the anti-apoptotic agent is an agent which inhibits (partially or fully) the activity of a Janus kinase (JAK). In some embodiments, the anti-apoptotic agent is an agent which inhibits (partially or fully) the activity of a Janus kinase 2 (JAK2). In some embodiments, the anti-apoptotic agent is VX-680, TG101348, TG101209, INCB018424, XL019, CEP-701, AT9283, or combinations thereof.

Bcl-2 Family

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

In some embodiments, the anti-apoptotic agent is Bcl-2. In certain instance, Bcl-2 partially or fully inhibits activation of a caspase (e.g. caspase 3 and caspase-6). In certain instances, treatment with, or agonism of, Bcl-2 (or a splice variant thereof) ameliorates ischemic damage to neural cells. In certain instances, treatment with, or agonism of, Bcl-2 (or a splice variant thereof) ameliorates cisplatin-induced apoptosis. In certain instances, treatment with, or agonism of, Bcl-2 (or a splice variant thereof) ameliorates neomycin-induced apoptosis. In certain instances, treatment with, or agonism of, Bcl-2 (or a splice variant thereof) ameliorates acoustic trauma-induced apoptosis.

In some embodiments, the anti-apoptotic agent is an artificial protein comprising at least a portion of an apoptosis modulating Bcl-2 polypeptide. In some embodiments, the apoptosis modulating member of the Bcl-2 family is Basal cell lymphoma-extra large (Bcl-XL). Bcl-XL is an apoptosis modulating member of the Bcl-2 family which is often over-expressed in cancer cells. In some embodiments, the artificial protein derived from Bcl-x(L) is FNK. In some embodiments, a fusion protein is constructed comprising FNK and the transduction domain (TAT) of the HIV/TAT protein construct. For the cDNA sequence of the FNK-TAT construct see U.S. Pat. No. 7,253,269 which is incorporated herein by reference for such disclosure. Administration of the FNK-TAT protein construct inhibits the induction of apoptosis in neurons and otic hair cells damaged by trauma.

In some embodiments, the anti-apoptotic agent is an agent that inhibits (partially or fully) the activity of Bax, BAD, Bak, Bok, or combinations thereof. In certain instances, a Bax polypeptide (alone or in combination with another polypeptide) forms a pore in the outer membrane of a cell's mitochondria. In certain instances, a Bak polypeptide (alone or in combinations with another polypeptide) forms a pore in the outer membrane of a cell's mitochondria. The formation of a pore in the mitochondria of a cell partially or fully results in the release of cytochrome c and other pro-apoptotic factors from the mitochondria (“mitochondrial outer membrane permeabilization”). In certain instances, mitochondrial outer membrane permeabilization, partially or fully results in the activation of multiple caspases. In certain instances, the activation of a caspase partially or fully results in the induction of apoptosis. In certain instances, BAD polypeptides bind to Bcl-2 and Bcl-xL. In certain instances, the binding of a BAD polypeptide to a Bc1-2 or Bcl-xL polypeptide inactivates the Bc1-2 and/or Bcl-xL polypeptide. In certain instances, the inactivation of a Bc1-2 or Bcl-xL polypeptide facilitates apoptosis induced by Bax and/or Bak.

In some embodiments, the anti-apoptotic agent is Bax inhibiting peptide V5 (also known as Bax inhibitor peptide V5); Bax channel blocker ((±)-1-(3,6-Dibromocarbazol-9-yl)-3-piperazin-1-yl-propan-2-ol); Bax inhibiting peptide P5 (also known as Bax inhibitor peptide P5); or combinations thereof. In some embodiments, the anti-apoptotic agent inhibits (partially or fully) the activity of Bak. In some embodiments, the anti-apoptotic agent inhibits (partially or fully) the activity of BAD.

FAS

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

Fas (also known as CD95, Apo-1, and TNFRSf6) is a receptor. In certain instances, when bound by its ligand, Fas forms a death inducing signaling complex (DISC). A DISC is composed of a trimer of Fas receptors bound by their ligands and several other polypeptides including, but not limited to, FADD, and caspase 8. In certain instances, a DISC is internalized into a cell. In certain instances, the binding of caspase-8 polypeptide to a FADD results in the activation of a caspase 8 polypeptide. In certain instances active caspase-8 polypeptide is released from a DISC into the cytosol of a cell. In certain instances, an active caspase-8 polypeptide cleaves other effector caspases resulting in DNA degradation, membrane blebbing, cell shrinkage, chromatin condensation, and nuclear and cytoplasmic fragmentation.

In some embodiments, the anti-apoptotic agent is an agent that inhibits (partially or fully) the activity of FAS. In some embodiments, the anti-apoptotic agent is Kp7-6; FAIM(S) (Fas apoptosis inhibitory molecule-short); FAIM(L) (Fas apoptosis inhibitory molecule-long); Fas:Fc; FAP-1; or combinations thereof. In some embodiments, the anti-apoptotic agent is an anti-Fas ligand antibody; an anti-Fas antibody; or combinations thereof. In some embodiments, the anti-Fas ligand antibody is NOK2; F2051; F1926; F2928; or combinations thereof. In some embodiments, the anti-Fas antibody is ZB4; Fas M3 mAb; or combinations thereof.

Akt

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

In certain instances, Akt1 (also known as Aktα) inhibits (alone or in combination with other polypeptides) apoptosis. In certain instances, Akt binds to PIP3 and/or PIP2. In certain instances, after binding to PIP3, Akt is phosphorylated by PDPK1, mTORC2, and DNA-PK. In certain instances, Akt regulates (alone or in combination with other polypeptides) apoptosis by binding and regulating, amongst other polypeptides, Nuclear Factor-κB, the Bcl-2 family, MDM2, FOXO1, GSK-3, Raf-1, ASK, Chk1, Bad, and MDM2.

In some embodiments, the anti-apoptotic agent is an agent that inhibits (partially or fully) the activity of Akt1. In some embodiments, the anti-apoptotic agent is a growth factor. In some embodiments, the growth factor is EGF.

PI3 Kinases

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

In certain instances, phosphoinositide 3-kinase (PI3 kinase) phosphorylates the 3 position hydroxyl group of the inositol ring of a phosphatidylinositol (e.g. phosphatidylinositol (3,4,5)-trisphosphate). In certain instances, a PI3 (e.g. p110α, p110δ and p110γ) activates PIP3 which binds to an AKT. In certain instances, an AKT bound to a PIP3 is phosphorylated by PDPK1, mTORC2, and DNA-PK. In certain instances, a phosphorylated AKT inhibits apoptosis.

In some embodiments, the anti-apoptotic agent is an agent that inhibits (partially or fully) the activity of PI3 kinases. In some embodiments, the anti-apoptotic agent is 740 Y—P; SC 3036 (KKHTDDGYMPMSPGVA); PI 3-kinase Activator (Santa Cruz Biotechnology, Inc.); or combinations thereof.

NF-kB

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

In certain instances, an NF-kB (nuclear factor-kappa B) transcription factor is formed from the homo-dimerization and hetero-dimerization of several subunits. The subunits include, but not limited to, NF-κB 1 (p50); NF-κB2 (p52); RelA (p65); RelB; and c-Rel. In certain instances, NF-kB is composed of a heterodimer of p50 and p65, or a heterodimer of p52 and p65. p65 contains the transactivation domains. In certain instances, inactive NF-kB is found in the cytosol and is bound by regulatory proteins (e.g. IkBa and IkBb).

In certain instances, a member of the NF-κB family is activated in response to (amongst other triggers) cytokines, LPS, UV radiation, shock (e.g. heat, or osmotic), oxidative stress, or combinations thereof. In certain instances, exposure to the aforementioned triggers leads to the phosphorylation of an IkB by IKK. In certain instances, the phosphorylation of an IkB by IKK leads to the proteolytic degradation of IkB. In certain instances, the degradation of an IkB allows NF-kB to translocate to the nucleus where it binds to kB enhancer elements of target genes and induces transcription. In certain instances, an active NF-κB transcription factor inhibits apoptosis. In certain instances, an active NF-κB transcription factor inhibits the apoptosis modulating genes TRAF1 and TRAF2. In certain instances, an active NF-κB transcription factor promotes apoptosis.

Accordingly, some embodiments incorporate the use of agents that modulate an NF-kB transcription factor. In certain instances, the agent that modulates an NF-kB transcription factor is an antagonist, partial agonist, inverse agonist, neutral or competitive antagonist, allosteric antagonist, and/or orthosteric antagonist of NF-kB. In some embodiments, the agent that modulates an NF-kB transcription factor is an NF-kB transcription factor agonist, partial agonist, and/or positive allosteric modulator. In some embodiments, the NF-kB transcription factor agonist, partial agonist, and/or positive allosteric modulator is Pam₃Cys ((S)-(2,3-bis(palmitoyloxy)-(2RS)-propyl)-N-palmitoyl-(R)-Cys-(S)-Ser(S)-Lys4-OH, trihydrochloride); Act1 (NF-kB activator 1); or combinations thereof.

In some embodiments, the NF-kB agonist, partial agonist, and/or positive allosteric modulator is an IkB antagonist, partial agonist, inverse agonist, neutral or competitive antagonist, allosteric antagonist, and/or orthosteric antagonist. In some embodiments, the IkB antagonist, partial agonist, inverse agonist, neutral or competitive antagonist, allosteric antagonist, and/or orthosteric antagonist is an anti-IkB antibody.

In some embodiments, the agent that modulates an NF-kB transcription factor is an NF-kB transcription factor antagonist, partial agonist, inverse agonist, neutral or competitive antagonist, allosteric antagonist, and/or orthosteric antagonist. In some embodiments, the NF-kB transcription factor antagonist, partial agonist, inverse agonist, neutral or competitive antagonist, allosteric antagonist, and/or orthosteric antagonist is Acetyl-11-keto-b-Boswellic Acid; Andrographolide; Caffeic Acid Phenethyl Ester (CAPE); Gliotoxin; Isohelenin; NEMO-Binding Domain Binding Peptide (DRQIKIWFQNRRMKWKKTALDWSWLQTE); NF-kB Activation Inhibitor (6-Amino-4-(4-phenoxyphenylethylamino)quinazoline); NF-kB Activation Inhibitor II (4-Methyl-N1-(3-phenylpropyl)benzene-1,2-diamine); NF-kB Activation Inhibitor III (3-Chloro-4-nitro-N-(5-nitro-2-thiazolyl)-benzamide); NF-kB Activation Inhibitor IV ((E)-2-Fluoro-4′-methoxystilbene); NF-kB Activation Inhibitor V (5-Hydroxy-(2,6-diisopropylphenyl)-1H-isoindole-1,3-dione); NF-kB SN50 (AAVALLPAVLLALLAPVQRKRQKLMP); Oridonin; Parthenolide; PPM-18 (2-Benzoylamino-1,4-naphthoquinone); Ro106-9920; Sulfasalazine; TIRAP Inhibitor Peptide (RQIKIWFNRRMKWKKLQLRDAAPGGAIVS); Withaferin A; Wogonin; or combinations thereof.

In some embodiments, the agent that modulates an NF-kB transcription factor inhibits NF-kB activation by TNF. In some embodiments, the agent that modulates an NF-kB transcription factor is an antagonist, partial agonist, inverse agonist, neutral or competitive antagonist, allosteric antagonist, and/or orthosteric antagonist of TNF. In some embodiments, the agent that inhibits NF-kB activation by TNF is BAY 11-7082 ((E)3-[(4-Methylphenyl)sulfonyl]-2-propenenitrile); BAY 11-7085 ((E)3-[(4-t-Butylphenyl)sulfonyl]-2-propenenitrile); (E)-Capsaicin; or combinations thereof.

In some embodiments, the agent that modulates an NF-kB transcription factor is an IKK antagonist, partial agonist, inverse agonist, neutral or competitive antagonist, allosteric antagonist, and/or orthosteric antagonist. In some embodiments, the IKK antagonist, partial agonist, inverse agonist, neutral or competitive antagonist, allosteric antagonist, and/or orthosteric antagonist is Aurothiomalate (ATM or AuTM); Evodiamine; Hypoestoxide; IKK Inhibitor III (BMS-345541); IKK Inhibitor VII; IKK Inhibitor X; IKK Inhibitor II; IKK-2 Inhibitor IV; IKK-2 Inhibitor V; IKK-2 Inhibitor VI; IKK-2 Inhibitor (SC-514); IkB Kinase Inhibitor Peptide; IKK-3 Inhibitor IX; or combinations thereof.

In some embodiments, the NF-kB antagonist, partial agonist, inverse agonist, neutral or competitive antagonist, allosteric antagonist, and/or orthosteric antagonist is an IKK agonist, partial agonist, and/or positive allosteric modulator.

p38

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

In certain instances, p38 (a mitogen-activated protein kinases (MAPK)) induces (alone or in combination with other polypeptides) apoptosis in response to the presence of (amongst other triggers) cytokines, LPS, UV radiation, shock (e.g. heat, or osmotic), oxidative stress, or combinations thereof. In certain instances, an activated p38 polypeptide phosphorylates MAPKAP kinase 2, ATF-2, Mac, and MEF2. In certain instances, the phosphorylation of MAPKAP kinase 2, ATF-2, Mac, and/or MEF2 leads to apoptosis. In certain instances, apoptosis is decreased in a cochlear culture exposed to an ototoxic agent (e.g. neomycin and/or cisplatin) when the culture is first treated with the p38 inhibitor SB-203580.

Accordingly, some embodiments incorporate the use of agents that modulate p38. In some embodiments, the agent that modulates p38 is a p38 antagonist, partial agonist, inverse agonists, neutral or competitive antagonists, allosteric antagonists, and/or orthosteric antagonists. In some embodiments, the p38 antagonist, partial agonist, inverse agonists, neutral or competitive antagonist, allosteric antagonist, and/or orthosteric antagonist is ARRY-797 (Array BioPharma); SB-220025 (5-(2-Amino-4-pyrimidinyl)-4-(4-fluorophenyl)-1-(4-piperidinlyl)imidazole); SB-239063 (trans-4-[4-(4-Fluorophenyl)-5-(2-methoxy-4-pyrimidinyl)-1H-imidazol-1-yl]cyclohexanol); SB-202190 (4-(4-Fluorophenyl)-2-(4-hydroxyphenyl)-5-(4-pyridyl) 1H-imidazole); JX-401 (-[2-Methoxy-4-(methylthio)benzoyl]-4-(phenylmethyl)piperidine); PD-169316 (4-(4-Fluorophenyl)-2-(4-nitrophenyl)-5-(4-pyridyl)-1H-imidazole); SKF-86002 (6-(4-Fluorophenyl)-2,3-dihydro-5-(4-pyridinyl)imidazo[2,1-b]thiazole dihydrochloride); SB-200646 (N-(1-Methyl-1H-indol-5-yl)-N′-3-pyridinylurea); CMPD-1 (2′-Fluoro-N-(4-hydroxyphenyl)-[1,1′-biphenyl]-4-butanamide); EO-1428 ((2-Methylphenyl)-[4-[(2-amino-4-bromophenyl)amino]-2-chlorophenyl]methanone); SB-253080 (4-[5-(4-Fluorophenyl)-2-[4-(methylsulfonyl)phenyl]-1H-imidazol-4-yl]pyridine); SD-169 (1H-Indole-5-carboxamide); SB-203580 (4-(4-Fluorophenyl)-2-(4-methylsulfinyl phenyl)-5-(4-pyridyl) 1H-imidazole); or combinations thereof.

Ghrelin

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

In certain embodiments, ghrelin is a ligand that binds to the growth hormone secretagogue receptor (GHS-R). In certain instances, exposure to ghrelin inhibits apoptosis in cardiomyocytes, endothelial cells, adipocyte, adrenal zona glomerulosa cells, pancreatic β-cells, osteoblastic MC3T3-E1 cells, intestinal epithelial cells, and/or hypothalamic neurons. In certain instances, exposure to ghrelin leads to activation of ERK1/2. In certain instances, exposure to ghrelin decreases the production of reactive oxygen species. In certain instances, exposure to ghrelin stabilizes mitochondrial transmembrane potential. In addition, exposure to ghrelin-treated leads to an increase in Bcl-2/Bax ratio, and antagonism of caspase-3.

Accordingly, some embodiments incorporate the use of agents that modulate ghrelin. In certain instances, the agent that modulates ghrelin is an antagonist, partial agonist, inverse agonist, neutral or competitive antagonist, allosteric antagonist, and/or orthosteric antagonist of ghrelin. In some embodiments, the agent that modulates ghrelin is a ghrelin agonist, partial agonist, and/or positive allosteric modulator. In some embodiments, the ghrelin agonist, partial agonist, and/or positive allosteric modulator is TZP-101 (Tranzyme Pharma); TZP-102 (Tranzyme Pharma); GHRP-6 (growth hormone-releasing peptide-6); GHRP-2 (growth hormone-releasing peptide-2); EX-1314 (Elixir Pharmaceuticals); MK-677 (Merck); L-692,429 (Butanamide, 3-amino-3-methyl-N-(2,3,4,5-tetrahydro-2-oxo-1-((2′-(1H-tetrazol-5-yl)(1,1′-biphenyl)-4-yl)methyl)-1H-1-benzazepin-3-yl)-, (R)—); EP1572 (Aib-DTrp-DgTrp-CHO); diltiazem; metabolites of diltiazem; or combinations thereof.

BRE

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

Accordingly, some embodiments incorporate the use of apoptosis modulating polypeptides, agonist, partial agonist, and/or positive allosteric modulators of apoptosis modulating polypeptides, or combinations thereof. In some embodiments, the apoptosis modulating polypeptide is BRE (Brain and Reproductive organ-Expressed protein). In certain instances, BRE is a receptor antagonist. In certain instances, BRE is a TNF-R1 antagonist. In certain instances, BRE is a Fas antagonist. In certain instances, the antagonism of TNF-R1 and/or Fas by BRE partially or fully inhibits the downstream activation of a caspase. In certain instances, inhibiting the activation of a caspase partially or fully inhibits apoptosis. In certain instances, the binding of BRE to TNF-R1 and/or Fas partially or fully inhibits the mitochondrial apoptotic pathway.

Calcium Channel Blockers

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

Accordingly, some embodiments incorporate the use of agents which antagonize the opening of Ca²⁺ channels. The transcription of multiple pro-apoptotic genes and apoptosis modulating genes are controlled by Ca²⁺ ion levels. Additionally, Ca²⁺ ions are integral to the activation of multiple enzymes including, but not limited to, phospholipases, endonucleases, and proteases. If these enzymes are over-activated they damage the cytoskeleton, plasma membrane, mitochondria, and DNA of the neuron. If the damage is significant, the neuron will undergo apoptosis. In some embodiments, the anti-apoptotic agent or pro-apoptotic agent is an antagonist, partial agonist, inverse agonist, neutral or competitive antagonist, allosteric antagonist, and/or orthosteric antagonist of a Ca²⁺ channel blocker. In some embodiments, the Ca²⁺ channel blockers are verapamil, nimodipine, diltiazem, omega-conotoxin, GVIA, amlodipine, felodipine, lacidipine, mibefradil, NPPB (5-Nitro-2-(3-phenylpropylamino)benzoic Acid), flunarizine, or combinations thereof.

Apolipoproteins

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

In some embodiments, the anti-apoptotic agent is an agent that promotes the activity of an apolipoprotein. In some embodiments, the anti-apoptotic agent is ApoE, agonists of ApoE, mimics of ApoE, homologues of ApoE, or combinations thereof. In some embodiments, the anti-apoptotic agent is ApoA, agonists of ApoA, mimics of ApoA, homologues of ApoA, or combinations thereof. In some embodiments, the anti-apoptotic agent is ApoB, agonists of ApoB, mimics of ApoB, homologues of ApoB, or combinations thereof. In some embodiments, the anti-apoptotic agent is ApoC, agonists of ApoC, mimics of ApoC, homologues of ApoC, or combinations thereof. In some embodiments, the anti-apoptotic agent is ApoD, agonists of ApoD, mimics of ApoD, homologues of ApoD, or combinations thereof. In some embodiments, the anti-apoptotic agent is ApoH, agonists of ApoH, mimics of ApoH, homologues of ApoH, or combinations thereof.

Erythropoietin

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

Accordingly, some embodiments incorporate the use of agents which regulate the activity of anti-apoptosis genes. In some embodiments, the agent which regulates the activity of anti-apoptosis genes is erythropoietin (EPO). EPO is a glycoprotein that, upon binding to its receptor, activates the JAK2 cascade. This eventually results in the activation of multiple apoptosis modulating genes. EPO receptors are found in the cytoplasm of the inner and outer phalangeal cells, the inner sulcus cells, cells supporting the organ of Corti, and spiral ganglion neurons.

Treatment with exogenous EPO results in a decrease in the number of cells undergoing apoptosis. It also ameliorates the damage induced by acoustic trauma and ischemia, and it protects neurons from glutamate-induced excitotoxicity. Thus, EPO protects neurons and otic hair cells from apoptosis, and/or damage-induced apoptosis. Accordingly, in some embodiments, the anti-apoptotic agent or pro-apoptotic agent is an antagonist, partial agonist, inverse agonist, neutral or competitive antagonist, allosteric antagonist, and/or orthosteric antagonist of erythropoietin.

HO-1

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

The expression of HO-1 inhibits the induction of apoptosis. Accordingly, some embodiments incorporate the use of agents which modulate the activity of heme-oxygenase-1 (HO-1) activity. In some embodiments, the modulator of HO-1 is an antagonist, partial agonist, inverse agonist, neutral or competitive antagonist, allosteric antagonist, and/or orthosteric antagonist of HO-1. In some embodiments, the modulator of HO-1 is a HO-1 agonist, partial agonist, and/or positive allosteric modulator. In some embodiments, the agonist, partial agonist, and/or positive allosteric modulator of HO-1 is piperine, hemin, and/or brazilin.

Caspase

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

In some embodiments, an antagonist, partial agonist, inverse agonist, neutral or competitive antagonist, allosteric antagonist, and/or orthosteric antagonist of a caspase target, including but not limited to caspase-8 and/or caspase-9, is incorporated. Some embodiments incorporate the use of caspase inhibitors. Caspases are proteases some of which mediate apoptosis. Both caspase 8 and caspase 9 are found in hair cells subjected to acoustic trauma, aminoglycoside treatment, and cisplatin treatment. When vestibular hair cells are treated with a caspase antagonist, partial agonist, inverse agonists, neutral or competitive antagonists, allosteric antagonists, and/or orthosteric antagonist following treatment with neomycin cell survival is maintained. In some embodiments, the caspase inhibitor is z-VAD-FMK (Benzyloxycarbonyl-Val-Ala-Asp(OMe)-fluoromethylketone); z-LEHD-FMK (benzyloxycarbonyl-Leu-Glu(OMe)-His-Asp(OMe)-fluoromethylketone); B-D-FMK (boc-aspartyl(Ome)-fluoromethylketone); Ac-LEHD-CHO (N-acetyl-Leu-Glu-His-Asp-CHO); Ac-IETD-CHO (N-acetyl-Ile-Glu-Thr-Asp-CHO); z-IETD-FMK (benzyloxycarbonyl-Ile-Glu(OMe)-Thr-Asp(OMe)-fluoromethy lketone); FAM-LEHD-FMK (benzyloxycarbonyl Leu-Glu-His-Asp-fluoromethyl ketone); FAM-LETD-FMK (benzyloxycarbonyl Leu-Glu-Thr-Asp-fluoromethyl ketone); Q-VD-OPH (Quinoline-Val-Asp-CH₂—O-Ph); or combinations thereof.

Inhibitor of Apoptosis Protein (IAP)

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

In some embodiments, an antagonist, partial agonist, inverse agonist, neutral or competitive antagonist, allosteric antagonist, and/or orthosteric antagonist of an apoptosis modulating polypeptide is incorporated. Some embodiments incorporate the use of apoptosis modulating polypeptides, agonist, partial agonist, and/or positive allosteric modulators of apoptosis modulating polypeptides, or combinations thereof. In some embodiments, the apoptosis modulating polypeptides is a member of the Inhibitor of Apoptosis Protein (IAP) family (e.g. XIAP; cIAP-1; cIAP-2; ML-IAP; ILP-2; NAIP; Survivin; Bruce; and IAPL-3). In certain instances, treatment with, or agonism of, XIAP ameliorates the development and/or progression of presbycusis. In certain instances, treatment with, or agonism of, XIAP ameliorates the loss of hearing in high-frequency ranges. In certain instances, treatment with, or agonism of, XIAP ameliorates gentamicin-induced hearing loss.

In certain instances, a member of the IAP family antagonizes a caspase (e.g. caspase 3, caspase 7, caspase 8, and caspase 9). In certain instances, XIAP antagonizes caspase 3, caspase, 7, and caspase 9. In certain instances, cIAP-1 antagonizes caspase 3, and caspase, 7. In certain instances, cIAP-2 antagonizes caspase 3, and caspase, 7. In certain instances, ML-IAP antagonizes caspase 3, and caspase 9. In certain instances, ILP-2 antagonizes caspase 9. In certain instances, NIAP antagonizes caspase 3, and caspase, 7. In certain instances, survivin antagonizes caspase 9. In certain instances, the antagonism of a caspase partially or fully inhibits apoptosis. In certain instances, a member of the IAP family catalyzes ubiquination of a caspase. In certain instances, XIAP catalyzes the ubiquination of a caspase. In certain instances, cIAP-1 catalyzes the ubiquination of a caspase. In certain instances, cIAP-2 catalyzes the ubiquination of a caspase (e.g. caspase 3 and caspase 7).

In some embodiments, the member of the IAP family is XIAP(X-linked IAP); cIAP-1 (cellular IAP-1); cIAP-2 (cellular IAP-2); ML-IAP (melanoma IAP); ILP-2 (IAP-like protein); NAIP (neuronal apoptosis-inhibitory protein); Survivin; Bruce; IAPL-3; or combinations thereof. In some embodiments, the IAP is XIAP. In some embodiments, the IAP is administered before, after, or simultaneously with a second polypeptide. In some embodiments, Survivin is administered before, after, or simultaneously with hepatitis B X-interacting protein (HBXIP). In some embodiments, ILP-2 is administered with a binding partner. In certain instances, the binding partner stabilizes ILP-2.

Fortilin

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

Accordingly, some embodiments incorporate the use of an antagonist, partial agonist, inverse agonist, neutral or competitive antagonist, allosteric antagonist, and/or orthosteric antagonist of an apoptosis modulating polypeptide. In some embodiments an apoptosis modulating polypeptides, agonist, partial agonist, and/or positive allosteric modulators of apoptosis modulating polypeptides, or combinations thereof are employed. In some embodiments, the apoptosis modulating polypeptide is fortilin. In certain instances, fortilin binds Ca²⁺. In certain instances, Ca²⁺ mediates the transcription of multiple pro-apoptotic genes. In certain instances, the binding of Ca²⁺ by fortilin partially or fully inhibits Ca²⁺-mediated transcription of pro-apoptotic genes.

Calpain

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

Accordingly, some embodiments incorporate the use of one or more antagonists, partial agonists, inverse agonists, neutral or competitive antagonists, allosteric antagonists, and/or orthosteric antagonists of calpain. Calpains are calcium-dependent, non-lysosomal cystein proteases. They participate in the apoptosis of cells. Leupeptine, an inhibitor of calpain, protects neurons and otic hair cells from aminoglycoside ototoxicity. Further, calpains are often found in the neurons and/or hair cells of the auris after cisplatin treatment or acoustic trauma. In some embodiments, the inhibitor of calpains is leupeptine; PD-150606 (3-(4-Iodophenyl)-2-mercapto-(Z)-2-propenoic acid); MDL-28170 (Z-Val-Phe-CHO); calpeptin; acetyl-calpastatin; MG 132 (N-[(phenylmethoxy)carbonyl]-L-leucyl-N-[(1S)-1-formyl-3-methylbutyl]-L-leucinamide); MYODUR; BN 82270 (Ipsen); BN 2204 (Ipsen); or combinations thereof.

p53

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

Accordingly, some embodiments incorporate the use of one or more antagonist, partial agonist, inverse agonists, neutral or competitive antagonists, allosteric antagonists, and/or orthosteric antagonists of p53. p53 is a transcription factor which regulates cell cycle and initiates apoptosis in damaged cells. In some embodiments, the antagonist, partial agonist, inverse agonists, neutral or competitive antagonists, allosteric antagonists, and/or orthosteric antagonists of p53 is an siRNA molecule, AHLi-11 (Quark Pharmaceuticals), an mdm2 protein, pifithrin-α(1-(4-Methylphenyl)-2-(4,5,6,7-tetrahydro-2-imino-3 (2H)-benzothiazolyl)ethanone), analogs thereof, or combinations thereof.

Heat Shock Proteins

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

In some embodiments, the anti-apoptotic agent is an agent that promotes the activity of heat shock proteins. In some embodiments, the anti-apoptotic agent is a heat shock protein. In some embodiments, the anti-apoptotic agent is an Hsp, an agonist of an Hsp, or a homologue or mimic thereof. In some embodiments, the anti-apoptotic agent is Hsp70, Hsp72, BiP (or Grp78), mtHsp70 (or Grp75), Hsp70-1b, Hsp70-1L, Hsp70-2, Hsp70-4, Hsp70-6, Hsp70-7, Hsp70-12a, Hsp70-14, Hsp10, Hsp27, Hsp40, Hsp60, Hsp90, Hsp104, Hsp110, Grp94, or combinations thereof.

Trefoil Factors

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

In certain instances, a trefoil factor induces activation of NF-κB. In certain instances, the activation of NF-κB inhibits apoptosis.

In some embodiments, the anti-apoptotic agent is an agent that promotes the activity of trefoil factor. In some embodiments, the anti-apoptotic agent is a trefoil factor. In some embodiments, the anti-apoptotic agent is a trefoil factor, an agonist of a trefoil factor, or a homologue or mimic thereof. In some embodiments, the anti-apoptotic agent is TFF1, TFF2, TFF3 or combinations thereof.

Sirtuin Modulators

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

Accordingly, some embodiments incorporate the use of one or more antagonists, partial agonists, inverse agonists, neutral or competitive antagonists, allosteric antagonists, and/or orthosteric antagonists of sirtuins. The sirtuins (or Sir2 proteins) comprise class III of the histone deacetylases (HDACs). There are seven members of the family: Sirt1, Sirt2, Sirt3, Sirt4, Sirt5, Sirt6, and Sirt7. Agonism of Sirt1 can prevent apoptosis by deacetylating the pro-apoptotic genes p53 and Ku-70. In some embodiments, the agonist, partial agonist, and/or positive allosteric modulator of sirtuin activity is a stilbene, flavone, isoflavone, flavanone, catechin, free radical protective compound, isonicotinamide, dipyridamole, ZM 336372 (3-(dimethylamino)-N-[3-[(4-hydroxybenzoyl)-amino]-4-methylphenyl]benzamide), camptothecin, coumestrol, nordihydroguaiaretic acid, esculetin, SRT-1720 (Sirtris), SRT-1460 (Sirtris), SRT-2183 (Sirtris), analogs thereof, or combinations thereof.

In some embodiments, the agonist, partial agonist, and/or positive allosteric modulator of sirtuins is a stilbene. In some embodiments, the stilbene is trans-stilbene, cis-stilbene, resveratrol, piceatannol, rhapontin, deoxyrhapontin, butein, or combinations thereof.

In some embodiments, the agent which modulates sirtuin catalyzed deacetylation reactions is a chalcone. In some embodiments, the chalcone is chalcon; isoliquirtigen; butein; 4,2′,4′-trihydroxychalcone; 3,4,2′,4′,6′-pentahydroxychalcone; or combinations thereof.

In some embodiments, the agent which modulates sirtuin catalyzed deacetylation reactions is a flavone. In some embodiments, the flavone is flavone, morin, fisetin; luteolin; quercetin; kaempferol; apigenin; gossypetin; myricetin; 6-hydroxyapigenin; 5-hydroxyflavone; 5,7,3′,4′,5′-pentahydroxyflavone; 3,7,3′,4′,5′-pentahydroxyflavone; 3,6,3′,4′-tetrahydroxyflavone; 7,3′,4′,5′-tetrahydroxyflavone; 3,6,2′,4′-tetrahydroxyflavone; 7,4′-dihydroxyflavone; 7,8,3′,4′-tetrahydroxyflavone; 3,6,2′,3′-tetrahydroxyflavone; 4′-hydroxyflavone; 5-hydroxyflavone; 5,4′-dihydroxyflavone; 5,7-dihydroxyflavone; or combinations thereof.

In some embodiments, the agent which modulates sirtuin catalyzed deacetylation reactions is an isoflavone. In some embodiments, the isoflavone is daidzein, genistein, or combinations thereof.

In some embodiments, the agent which modulates sirtuin catalyzed deacetylation reactions is a flavanone. In some embodiments, the flavanone is naringenin; flavanone; 3,5,7,3′,4′-pentahydroxyflavanone; or combinations thereof.

In some embodiments, the agent which modulates sirtuin catalyzed deacetylation reactions is an anthocyanidin. In some embodiments, the anthocyanidin is pelargonidin chloride, cyanidin chloride, delphinidin chloride, or combinations thereof.

In some embodiments, the agent which modulates sirtuin catalyzed deacetylation reactions is a catechin. In some embodiments, the catechin is (−)-epicatechin (Hydroxy Sites: 3,5,7,3′,4′); (−)-catechin (Hydroxy Sites: 3,5,7,3′,4′); (−)-gallocatechin (Hydroxy Sites: 3,5,7,3′,4′,5′) (+)-catechin (Hydroxy Sites: 3,5,7,3′,4′); (+)-epicatechin (Hydroxy Sites: 3,5,7,3′,4′); or combinations thereof.

In some embodiments, the agent which modulates sirtuin catalyzed deacetylation reactions is a free radical protective compound. In some embodiments, the free radical protective compound is Hinokitiol (b-Thujaplicin; 2-hydroxy-4-isopropyl-2,4,6-cycloheptatrien-1-one); L-(+)-Ergothioneine ((S)-a-Carboxy-2,3-dihydro-N,N,N-trimethyl-2-thioxo-1H-imidazole4-ethanaminium inner salt); Caffeic Acid Phenyl Ester; MCI-186 (3-Methyl-1-phenyl-2-pyrazolin-5-one); HBED (N,N′-Di-(2-hydroxybenzyl)ethylenediamine-N,N′-diacetic acidH2O); Ambroxol (trans-4-(2-Amino-3,5-dibromobenzylamino)cyclohexane-HCl; and U-83836E ((−)-2-((4-(2,6-di-1-Pyrrolidinyl-4-pyrimidinyl)-1-piperzainyl)methyl)-3,4-dihydro-2,5,7,8-tetramethyl-2H-1-benzopyran-6-ol2HCl); or combinations thereof.

In some embodiments, the nicotinamide binding antagonist is isonicotinamide or an analog of isonicotinamide. In some embodiments, the analog of isonicotinamide is β-1′-5-methyl-nicotinamide-2′-deoxyribose; β-D-1′-5-methyl-nico-tinamide-2′-deoxyribofuranoside; β-1′-4,5-dimethyl-nicotinamide-2′-de-oxyribose; or β-D-1′-4,5-dimethyl-nicotinamide-2′-deoxyribofuranoside. For additional analogs of isonicotinamide see U.S. Pat. Nos. 5,985,848; 6,066,722; 6,228,847; 6,492,347; 6,803,455; and U.S. Patent Publication Nos. 2001/0019823; 2002/0061898; 2002/0132783; 2003/0149261; 2003/0229033; 2003/0096830; 2004/0053944; 2004/0110772; and 2004/0181063, which are hereby incorporated by reference for that disclosure.

Src

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

Inhibition of Src pp60^{c-src} modulates apoptosis. Accordingly, some embodiments incorporate the use of one or more antagonists, partial agonists, inverse agonists, neutral or competitive antagonists, allosteric antagonists, and/or orthosteric antagonists of the Src family of protein kinases. The Src family is a family of non-receptor protein kinases. Examples of Src kinases found in vertebrates include, but are not limited to, Src, Yes, Fgr, Yrk, Fyn, Lyn, Hck, Lck and Blk. They catalyze the phosphorylation of proteins by transferring a phosphate from ATP to the free hydroxyl group on a serine, threonine, or tyrosine. By way of non-limiting example, targets of Src kinase catalyzed phosphorylation include vinculin, cortactin, talin, paxillin, FAK, tensin, ezrin, p130cas, β- and γ-catenin, ZO-1, occludin, p120ctn, connexin 43, nectin-2 delta. The Src kinases consist of an N-terminal SH3 domain, a central SH2 domain, and the tyrosine kinase domain. The binding of a ligand to the SH2 and SH3 domains induces a conformational change which inhibits the activity of the Src kinase.

In some embodiments, the Src is pp60^{c-src}. In some embodiments, the Src antagonist, partial agonist, inverse agonist, neutral or competitive antagonist, allosteric antagonist, and/or orthosteric antagonist is 1-Naphthyl PP1(1-(1,1-Dimethylethyl)-3-(1-naphthalenyl)-1H-pyrazolo[3,4-d]pyrimidin-4-amine); Lavendustin A (5-[[(2,5-Dihydroxyphenyl)methyl][(2-hydroxyphenyl)methyl]amino]-2-hydroxybenzoic acid); MNS (3,4-Methylenedioxy-b-nitrostyrene); PP1(1-(1,1-Dimethylethyl)-1-(4-methylphenyl)-1H-pyrazolo[3,4-d]pyrimidin-4-amine); PP2 (3-(4-chlorophenyl) 1-(1,1-dimethylethyl)-1H-pyrazolo[3,4-d]pyrimidin-4-amine); KX1-004 (Kinex); KX1-005 (Kinex); KX1-136 (Kinex); KX1-174 (Kinex); KX1-141 (Kinex); KX2-328 (Kinex); KX1-306 (Kinex); KX1-329 (Kinex); KX2-391 (Kinex); KX2-377 (Kinex); ZD4190 (Astra Zeneca; N-(4-bromo-2-fluorophenyl)-6-methoxy-7-(2-(1H-1,2,3-triazol-1-yl)ethoxy)quinazolin-4-amine); AP22408 (Ariad Pharmaceuticals); AP23236 (Ariad Pharmaceuticals); AP23451 (Ariad Pharmaceuticals); AP23464 (Ariad Pharmaceuticals); AZD0530 (Astra Zeneca); AZM475271 (M475271; Astra Zeneca); Dasatinib (N-(2-chloro-6-methylphenyl)-2-(6-(4-(2-hydroxyethyl)-piperazin-1-yl)-2-methylpyrimidin-4-ylamino) thiazole-5-carboxamide); GN963 (trans-4-(6,7-dimethoxyquinoxalin-2-ylamino)cyclohexanol sulfate); Bosutinib (4-((2,4-dichloro-5-methoxyphenyl)amino)-6-methoxy-7-(3-(4-methyl-1-piperazinyl)propoxy)-3-quinolinecarbonitrile); or combinations thereof. For disclosure of additional antagonists, partial agonists, inverse agonists, neutral or competitive antagonists, allosteric antagonists, and/or orthosteric antagonists of the Src family of kinases, see U.S. Pub. No. 2006/0172971, which is hereby incorporated by reference for those disclosures.

RNAi

Contemplated for use with the formulations disclosed herein are agents that protect neurons and otic hair cells from apoptosis (i.e., an anti-apoptotic agent). Further, contemplated for use with the formulations disclosed herein are agents that induce apoptosis in neurons and otic hair cells (i.e., a pro-apoptotic agent). Accordingly, some embodiments incorporate the use of an anti-apoptotic agent. Alternatively, some embodiments incorporate the use of a pro-apoptotic agent.

In some embodiments, where inhibition or down-regulation of a target is desired (e.g. genes in the MAPK/JNK cascade, caspase genes, Src genes, calpain genes, Ca²⁺ channel genes), RNA interference may be utilized. In some embodiments, the agent that inhibits or down-regulates the target is an siRNA molecule. In certain instances, the siRNA molecule inhibits the transcription of a target by RNA interference (RNAi). In some embodiments, a double stranded RNA (dsRNA) molecule with sequences complementary to a target is generated (e.g., by PCR). In some embodiments, a 20-25 bp siRNA molecule with sequences complementary to a target is generated. In some embodiments, the 20-25 bp siRNA molecule has 2-5 bp overhangs on the 3′ end of each strand, and a 5′ phosphate terminus and a 3′ hydroxyl terminus. In some embodiments, the 20-25 bp siRNA molecule has blunt ends. For techniques for generating RNA sequences see Molecular Cloning: A Laboratory Manual, second edition (Sambrook et al., 1989) and Molecular Cloning: A Laboratory Manual, third edition (Sambrook and Russel, 2001), jointly referred to herein as “Sambrook”); Current Protocols in Molecular Biology (F. M. Ausubel et al., eds., 1987, including supplements through 2001); Current Protocols in Nucleic Acid Chemistry John Wiley & Sons, Inc., New York, 2000) which are hereby incorporated by reference for such disclosure.

In some embodiments, the dsRNA or siRNA molecule is incorporated into a controlled-release auris-acceptable microsphere or microparticle, hydrogel, liposome, or thermoreversible gel. In some embodiments, the auris-acceptable microsphere, hydrogel, liposome, paint, foam, in situ forming spongy material, nanocapsule or nanosphere or thermoreversible gel is injected into the inner ear. In some embodiments, the auris-acceptable microsphere or microparticle, hydrogel, liposome, or thermoreversible gel. In some embodiments, the auris-acceptable microsphere, hydrogel, liposome, paint, foam, in situ forming spongy material, nanocapsule or nanosphere or thermoreversible gel is injected into the cochlea, the organ of Corti, the vestibular labyrinth, or a combination thereof.

In certain instances, after administration of the dsRNA or siRNA molecule, cells at the site of administration (e.g. the cells of cochlea, organ of Corti, and/or the vestibular labyrinth) are transformed with the dsRNA or siRNA molecule. In certain instances following transformation, the dsRNA molecule is cleaved into multiple fragments of about 20-25 bp to yield siRNA molecules. In certain instances, the fragments have about 2 bp overhangs on the 3′ end of each strand.

In certain instances, a siRNA molecule is divided into two strands (the guide strand and the anti-guide strand) by an RNA-induced Silencing Complex (RISC). In certain instances, the guide strand is incorporated into the catalytic component of the RISC (i.e. argonaute). In certain instances, the guide strand binds to a complementary target mRNA sequence. In certain instances, the RISC cleaves the target mRNA. In certain instances, the expression of the target gene is down-regulated.

In some embodiments, a sequence complementary to a target is ligated into a vector. In some embodiments, the sequence is placed between two promoters. In some embodiments, the promoters are orientated in opposite directions. In some embodiments, the vector is contacted with a cell. In certain instances, a cell is transformed with the vector. In certain instances following transformation, sense and anti-sense strands of the sequence are generated. In certain instances, the sense and anti-sense strands hybridize to form a dsRNA molecule which is cleaved into siRNA molecules. In certain instances, the strands hybridize to form an siRNA molecule. In some embodiments, the vector is a plasmid (e.g., pSUPER; pSUPER.neo; pSUPER.neo+gfp).

In some embodiments, the vector is incorporated into a controlled-release auris-acceptable microsphere or microparticle, hydrogel, liposome, or thermoreversible gel. In some embodiments, the auris-acceptable microsphere, hydrogel, liposome, paint, foam, in situ forming spongy material, nanocapsule or nanosphere or thermoreversible gel is injected into the inner ear. In some embodiments, the auris-acceptable microsphere or microparticle, hydrogel, liposome, or thermoreversible gel. In some embodiments, the auris-acceptable microsphere, hydrogel, liposome, paint, foam, in situ forming spongy material, nanocapsule or nanosphere or thermoreversible gel is injected into the cochlea, the organ of Corti, the vestibular labyrinth, or a combination thereof.

In some embodiments, the compositions described herein have a concentration of active pharmaceutical ingredient, or pharmaceutically acceptable prodrug or salt thereof, between about 0.1 to about 70 mg/mL, between about 0.5 mg/mL to about 70 mg/mL, between about 0.5 mg/mL to about 50 mg/mL, between about 0.5 mg/mL to about 20 mg/mL, between about 1 mg to about 70 mg/mL, between about 1 mg to about 50 mg/mL, between about 1 mg/mL and about 20 mg/mL, between about 1 mg/mL to about 10 mg/mL, or between about 1 mg/mL to about 5 mg/mL, of the active agent, or pharmaceutically acceptable prodrug or salt thereof, by volume of the composition.

Antibodies

Contemplated for use with the formulations disclosed herein are agents that inhibit the growth of otic neoplasms. In some embodiments, the antibody inhibits the growth of blood vessels. In some embodiments, the antibody induces the death (e.g., apoptosis) of a neoplastic cell. In some embodiments, the agent is an antibody. In some embodiments, the antibody is an anti-CD-20 antibody, an anti-CD22 antibody, an anti-CD32b antibody, an anti-CD-33 antibody, an anti-CD40 antibody, an anti-CD52 antibody, an anti-EGFR antibody, an anti-VEGF antibody, an anti-HER2 receptor antibody, an anti-17-1A antibody, an anti-CCR4 antibody, an anti-IGF-IR antibody, an anti-CTLA-4 antibody, or combinations thereof. In some embodiments, the antibody is an anti-CD20 antibody. In some embodiments, the antibody is rituximab, tositumomab, ibritumomab, epratuzumab, alemtuzumab, ocrelizumab (PRO70769), veltuzumab (IMMU-106 or hA20), ofatumumab (HuMax-CD20 human IgG1 antibody or 2F2), HuMAB 7D8 (Genmab A/S), AME-133v (LY2469298, Applied Molecular Evolution), GA101 (R7159, Genentech), PRO131921 (Genentech), rhuMAb v114, Hex-hA20 (Immunomedics), BLX301 (BioLex), Bi20 (FBTA05, TRION Pharma), epratuzumab, lumiliximab, HuM195, alemtuzumab, cetuximab, panitumumab, bevacizumab, trastuzumab, edrecolomab, adecatumumab, KM2760, rhuCD40 mAb, Dacetuzumab (SGN40), CP-870,893 (Pfizer), HCD122 (Novartis/Xoma), CP-675,206 (Pfizer), CP-751,871 (Pfizer), or combinations thereof.

### Combination Therapy

In some embodiments, the compositions disclosed herein further comprise an additional therapeutic agent. In some embodiments, the additional therapeutic agent is an acidifying agent, an anesthetic, an analgesic, an antibiotic, antiemetic, an antifungal, an anti-microbial agent, an antipsychotic (especially those in the phenothiazine class), an antiseptic, an antiviral, an astringent, a chemotherapeutic agent, a collagen, a corticosteroid, a diuretic, a keratolytic agent, a nitric oxide synthase inhibitor, or combinations thereof.

### Acidifying Agents

Acidifying agents are optionally used in combination with the compositions disclosed herein. Acidifying agents lower the pH level of the vestibular environment making it unfavorable to most microbial growth. Acidifying agents include, but are not limited to, acetic acid.

### Anti-Emetic Agents

Anti-Emetic agents are optionally used in combination with the compositions disclosed herein. Anti-emetic agents include promethazine, prochlorperazine, trimethobenzamide, and triethylperazine. Other anti-emetic agents include 5HT3 antagonists such as dolasetron, granisetron, ondansetron, tropisetron, and palonosetron; and neuroleptics such as droperidol. Further anti-emetic agents include antihistamines, such as meclizine; phenothiazines such as perphenazine, and thiethyl perazine; dopamine antagonists, including domperidone, properidol, haloperidol, chlorpromazine, promethazine, prochlorperazine, metoclopramide or combinations thereof; cannabinoids, including dronabinol, nabilone, sativex, or combinations thereof; anticholinergics, including scopolamine; and steroids, including dexamethasone; trimethobenzamine, emetrol, propofol, muscimol, or combinations thereof.

### Antimicrobial Agents

Antimicrobial agents are also contemplated as useful with the compositions disclosed herein. Antimicrobial agents include agents that act to inhibit or eradicate microbes, including bacteria, fungi or parasites. Specific antimicrobial agents may be used to combat specific microbes. Accordingly, a skilled practitioner would know that antimicrobial agent would be relevant or useful depending on the microbe identified, or the symptoms displayed. Antimicrobial agents include antibiotics, antiviral agents, antifungal agents, and antiparasitic agents.

Antibiotics include, but are not limited to, amikacin, gentamicin, kanamycin, neomycin, netilmicin, streptomycin, tobramycin, paromomycin, geldanmycin, herbimycin, loracarbef, ertapenem, doripenem, imipenem, cilastatin, meropenem, cefadroxil, cefazolin, cefalotin, cefalexin, cefaclor, cefamandole, cefoxitin, defprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, teicoplanin, vancomycin, azithromycin, clarithromycin, dirithromycin, erythromycin, roxithromycin, troleandomycin, telithromycin, spectinomycin, aztreonam, amoxicillin, ampicillin, azlocillin, carbenicillin, cloxacillin, dicloxacillin, flucloxacillin, mezlocillin, meticillin, nafcillin, oxacillin, penicillin, piperacillin, ticarcillan, bacitracin, colistin, polymyxin B, ciprofloxacin, enoxacin, gatifloxacin, levofloxacin, lomefloxacin, moxifloxacin, norfloxacin, ofloxacin, trovfloxacin, mafenide, prontosil, sulfacetamide, sulfamethizole, sulfanimilimde, sulfsalazine, sulfsioxazole, trimethoprim, demeclocycline, doxycycline, minocycline, oxtetracycline, tetracycline, arsphenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinuspristin/dalfopristin, rifampin, timidazole, or combinations thereof.

Antiviral agents include, but are not limited to, acyclovir, famciclovir and valacyclovir. Other antiviral agents include abacavir, aciclovir, adfovir, amantadine, amprenavir, arbidol., atazanavir, artipla, brivudine, cidofovir, combivir, edoxudine, efavirenz, emtricitabine, enfuvirtide, entecavir, fomvirsen, fosamprenavir, foscarnet, fosfonet, ganciclovir, gardasil, ibacitabine, immunovir, idoxuridine, imiquimod, indinavir, inosine, integrase inhibitors, interferons, including interferon type III, interferon type II, interferon type I, lamivudine, lopinavir, loviride, MK-0518, maraviroc, moroxydine, nelfinavir, nevirapine, nexavir, nucleoside analogues, oseltamivir, penciclovir, peramivir, pleconaril, podophyllotoxin, protease inhibitors, reverse transcriptase inhibitors, ribavirin, rimantadine, ritonavir, saquinavir, stavudine, tenofovir, tenofovir disoproxil, tipranavir, trifluridine, trizivir, tromantadine, truvada, valganciclovir, vicriviroc, vidarabine, viramidine, zalcitabine, zanamivir, zidovudine, or combinations thereof.

Antifungal agents include, but are not limited to, ammolfine, utenafine, naftifine, terbinafine, flucytosine, fluconazole, itraconazole, ketoconazole, posaconazole, ravuconazole, voriconazole, clotrimazole, econazole, miconazole, oxiconazole, sulconazole, terconazole, tioconazole, nikkomycin Z, caspofungin, micafungin, anidulafungin, amphotericin B, liposomal nystastin, pimaricin, griseofulvin, ciclopirox olamine, haloprogin, tolnaftate, undecylenate, or combinations thereof. Antiparasitic agents may include amitraz, amoscanate, avermectin, carbadox, diethylcarbamizine, dimetridazole, diminazene, ivermectin, macrofilaricide, malathion, mitaban, oxamniquine, permethrin, praziquantel, prantel pamoate, selamectin, sodium stibogluconate, thiabendazole, or combinations thereof.

### Anti-Septic Agents

Anti-septic agents are also contemplated as useful with the compositions disclosed herein. Anti-septic agents include, but are not limited to, acetic acid, boric acid, gentian violet, hydrogen peroxide, carbamide peroxide, chlorhexidine, saline, mercurochrome, povidone iodine, polyhyroxine iodine, cresylate and aluminum acetate, and mixtures thereof.

### Astringents

Astringents are also contemplated as useful with the compositions disclosed herein. Astringents include, but are not limited to, isopropyl alcohol, ethanol and propylene glycol.

### Corticosteroids

Corticosteroids are also contemplated as useful with the compositions disclosed herein. Corticosteroids include, but are not limited to, hydrocortisone, prednisone, fluprednisolone, dexamethasone, betamethasone, betamethasone valerate, methylprednisolone, fluocinolone acetonide, flurandrenolone acetonide, fluorometholone, cortisone, prednisolone, alclometasone, amcinonide, betamethasone, clobetasol, clocortolone, desonide, desoximetasone, diflorasone, fluocinonide, flurandrenolide, fluticasone, halcinonide, halobetasol, mometasone, flumethasone, prednicarbate and triamcinolone, and mixtures thereof.

### Platelet Activating Factor Antagonists

Platelet activating factor antagonists are also contemplated for use in combination with the apoptosis modulating compositions disclosed herein. Platelet activating factor antagonists include, by way of example only, kadsurenone, phomactin G, ginsenosides, apafant (4-(2-chlorophenyl)-9-methyl-2 [3 (4-morpholinyl)-3-propanol-1-yl[6H-thieno[3,2-f[[1.2.4]triazolo]4,3-1]]1.4]diazepine), A-85783, BN-52063, BN-52021, BN-50730 (tetrahedra-4,7,8,10 methyl-1 (chloro-1 phenyl)-6 (methoxy-4 phenyl-carbamoyl)-9 pyrido[4′,3′-4,5]thieno[3,2-f]triazolo-1,2,4 [4,3-a]diazepine-1,4), BN 50739, SM-12502, RP-55778, Ro 24-4736, SR27417A, CV-6209, WEB 2086, WEB 2170, 14-deoxyandrographolide, CL 184005, CV-3988, TCV-309, PMS-601, TCV-309 or combinations thereof.

Presented below (Table 1) are examples of active agents contemplated for use with the compositions and devices disclosed herein. In some embodiments, one or more active agents disclosed in Table 1 are used in a composition or device described herein.

TABLE 1

| Auris Condition | Therapeutic Agent |
|---|---|
| Benign Paroxysmal | Diphenhydramine |
| Positional Vertigo | |
| Benign Paroxysmal | Lorazepam |
| Positional Vertigo | |
| Benign Paroxysmal | Meclizine |
| Positional Vertigo | |
| Benign Paroxysmal | Oldansetron |
| Positional Vertigo | |
| Hearing Loss | Estrogen |
| AIED | Etanercept (Enbrel) |
| AIED | GW3333 |
| AIED | Copaxone |
| Hearing Loss | Estrogen and progesterone |
| | (E + P) |
| Hearing Loss | Folic acid |
| Hearing Loss | Lactated Ringer's with |
| | 0.03% Ofloxacin |
| Hearing Loss | Methotrexate |
| Hearing Loss | N-acetyl cysteine |
| Meniere's Disease | Betahistine |
| Meniere's Disease | Sildenafil |
| Meniere's Disease | Tacrolimus |
| Middle Ear Effusion | Pneumonococcal vaccine |
| Otitis Externa | Diclofenac sodium; dexotc |
| Otitis Externa, Acute | AL-15469A/AL-38905 |
| Otitis Media | Amoxicillin/clavulanate |
| Otitis Media | Dornase alfa |
| Otitis Media | Echinacea purpurea |
| Otitis Media | Faropenem medoxomil |
| Otitis Media | Levofloxacin |
| Otitis Media | PNCRM9 |
| Otitis Media | Pneumococcal vaccine |
| Otitis Media | Telithromycin |
| Otitis Media | Zmax |
| Otitis Media with | Lansoprazole |
| Effusion | |
| Otitis Media, Acute | AL-15469A; AL-38905 |
| Otitis Media, Acute | Amoxicillin |
| Otitis Media, Acute | Amoxicillin-clavulanate |
| Otitis Media, Acute | Azithromycin |
| Otitis Media, Acute | Azithromycin SR |
| Otitis Media, Acute | Cefdinir |
| Otitis Media, Acute | Hyland's earache drops |
| Otitis Media, Acute | Montelukast |
| Otitis Media, Acute | Pneumonococcal vaccine |
| Otitis Media, Acute | AL-15469A/AL38905 |
| with Typanostomy | |
| Tubes | |
| Otitis Media, Chronic | Sulfamethoxazole- |
| | trimethoprim |
| Otitis Media, | Azithromycin |
| Suppurative | |
| Otitis Media, | Telithromycin |
| Suppurative | |
| Otosclerosis | Acetylcysteine |
| Ototoxicity | Aspirin |
| Tinnitus | Acamprosate |
| Tinnitus | Gabapentin |
| Tinnitus | Modafinil |
| Tinnitus | Neramexane |
| Tinnitus | Neramexane mesylate |
| Tinnitus | Piribedil |
| Tinnitus | Vardenafil |
| Tinnitus | Vestipitant + Paroxetine |
| Tinnitus | Vestiplitant |
| Tinnitus | Zinc sulfate |

In some embodiments, the additional therapeutic agent is an immediate release agent. In some embodiments, the additional therapeutic agent is a controlled-release agent.

### General Methods of Sterilization

Provided herein are otic compositions that ameliorate or lessen otic disorders described herein. Further provided herein, in some embodiments, are methods comprising the administration of said otic compositions. In some embodiments, the compositions or devices are sterilized. Included within the embodiments disclosed herein are means and processes for sterilization of a pharmaceutical composition or device disclosed herein for use in humans. The goal is to provide a safe pharmaceutical product, relatively free of infection causing micro-organisms. The U.S. Food and Drug Administration has provided regulatory guidance in the publication “Guidance for Industry: Sterile Drug Products Produced by Aseptic Processing” available at: http://www.fda.gov/cder/guidance/5882fnl.htm, which is incorporated herein by reference in its entirety.

As used herein, “sterilization” means a process used to destroy or remove microorganisms that are present in a product or packaging. Any suitable method available for sterilization of objects and compositions is contemplated for use with the compositions and devices disclosed herein. Available methods for the inactivation of microorganisms include, but are not limited to, the application of extreme heat, lethal chemicals, or gamma radiation. Disclosed herein, in some embodiments, are processes for the preparation of an otic therapeutic composition comprising subjecting the composition to a sterilization method selected from heat sterilization, chemical sterilization, radiation sterilization or filtration sterilization. The method used depends largely upon the nature of the device or composition to be sterilized. Detailed descriptions of many methods of sterilization are given in Chapter 40 of Remington: The Science and Practice of Pharmacy published by Lippincott, Williams & Wilkins, and is incorporated by reference with respect to this subject matter.

Sterilization by Heat

Many methods are available for sterilization by the application of extreme heat. One method is through the use of a saturated steam autoclave. In this method, saturated steam at a temperature of at least 121° C. is allowed to contact the object to be sterilized. The transfer of heat is either directly to the microorganism, in the case of an object to be sterilized, or indirectly to the microorganism by heating the bulk of an aqueous solution to be sterilized. This method is widely practiced as it allows flexibility, safety and economy in the sterilization process.

Dry heat sterilization is a method that is used to kill microorganisms and perform depyrogenation at elevated temperatures. This process takes place in an apparatus suitable for heating HEPA-filtered microorganism-free air to temperatures of at least 130-180° C. for the sterilization process and to temperatures of at least 230-250° C. for the depyrogenation process. Water to reconstitute concentrated or powdered compositions is also sterilized by autoclave. In some embodiments, the compositions described herein comprise micronized pharmaceutical that are sterilized by dry heating, e.g., heating for about 7-11 hours at internal powder temperatures of 130-140° C., or for 1-2 hours at internal temperatures of 150-180° C.

Chemical Sterilization

Chemical sterilization methods are an alternative for products that do not withstand the extremes of heat sterilization. In this method, a variety of gases and vapors with germicidal properties, such as ethylene oxide, chlorine dioxide, formaldehyde or ozone are used as the anti-apoptotic agent or pro-apoptotic agents. The germicidal activity of ethylene oxide, for example, arises from its ability to serve as a reactive alkylating agent. Thus, the sterilization process requires the ethylene oxide vapors to make direct contact with the product to be sterilized.

Radiation Sterilization

One advantage of radiation sterilization is the ability to sterilize many types of products without heat degradation or other damage. The radiation commonly employed is beta radiation or alternatively, gamma radiation from a ⁶⁰Co source. The penetrating ability of gamma radiation allows its use in the sterilization of many product types, including solutions, compositions and heterogeneous mixtures. The germicidal effects of irradiation arise from the interaction of gamma radiation with biological macromolecules. This interaction generates charged species and free radicals. Subsequent chemical reactions, such as rearrangements and cross-linking processes, result in the loss of normal function for these biological macromolecules. The compositions described herein are also optionally sterilized using beta irradiation.

Filtration

Filtration sterilization is a method used to remove but not destroy microorganisms from solutions. Membrane filters are used to filter heat-sensitive solutions. Such filters are thin, strong, homogenous polymers of mixed cellulosic esters (MCE), polyvinylidene fluoride (PVF; also known as PVDF), or polytetrafluoroethylene (PTFE) and have pore sizes ranging from 0.1 to 0.22 μm. Solutions of various characteristics are optionally filtered using different filter membranes. For example, PVF and PTFE membranes are well suited to filtering organic solvents while aqueous solutions are filtered through PVF or MCE membranes. Filter apparatus are available for use on many scales ranging from the single point-of-use disposable filter attached to a syringe up to commercial scale filters for use in manufacturing plants. The membrane filters are sterilized by autoclave or chemical sterilization. Validation of membrane filtration systems is performed following standardized protocols (Microbiological Evaluation of Filters for Sterilizing Liquids, Vol 4, No. 3. Washington, D.C.: Health Industry Manufacturers Association, 1981) and involve challenging the membrane filter with a known quantity (ca. 10^{7/}cm²) of unusually small microorganisms, such as Brevundimonas diminuta (ATCC 19146).

Pharmaceutical compositions are optionally sterilized by passing through membrane filters. Compositions comprising nanoparticles (U.S. Pat. No. 6,139,870) or multilamellar vesicles (Richard et al., International Journal of Pharmaceutics (2006), 312(1-2):144-50) are amenable to sterilization by filtration through 0.22 μm filters without destroying their organized structure.

In some embodiments, the methods disclosed herein comprise sterilizing the composition (or components thereof) by means of filtration sterilization. In another embodiment the auris-acceptable otic therapeutic agent composition comprises a particle wherein the particle composition is suitable for filtration sterilization. In a further embodiment said particle composition comprises particles of less than 300 nm in size, of less than 200 nm in size, of less than 100 nm in size. In another embodiment the auris-acceptable composition comprises a particle composition wherein the sterility of the particle is ensured by sterile filtration of the precursor component solutions. In another embodiment the auris-acceptable composition comprises a particle composition wherein the sterility of the particle composition is ensured by low temperature sterile filtration. In a further embodiment, low temperature sterile filtration is carried out at a temperature between 0 and 30° C., between 0 and 20° C., between 0 and 10° C., between 10 and 20° C., or between 20 and 30° C.

In another embodiment, is a process for the preparation of an auris-acceptable particle composition comprising: filtering the aqueous solution containing the particle composition at low temperature through a sterilization filter; lyophilizing the sterile solution; and reconstituting the particle composition with sterile water prior to administration. In some embodiments, a composition described herein is manufactured as a suspension in a single vial composition containing the micronized active pharmaceutical ingredient. A single vial composition is prepared by aseptically mixing a sterile poloxamer solution with sterile micronized active ingredient (e.g., PD98059) and transferring the composition to sterile pharmaceutical containers. In some embodiments, a single vial containing a composition described herein as a suspension is resuspended before dispensing and/or administration.

In specific embodiments, filtration and/or filling procedures are carried out at about 5° C. below the gel temperature (T_{gel}) of a composition described herein and with viscosity below a theoretical value of 100 cP to allow for filtration in a reasonable time using a peristaltic pump.

In another embodiment the auris-acceptable otic therapeutic agent composition comprises a nanoparticle composition wherein the nanoparticle composition is suitable for filtration sterilization. In a further embodiment the nanoparticle composition comprises nanoparticles of less than 300 nm in size, of less than 200 nm in size, or of less than 100 nm in size. In another embodiment the auris-acceptable composition comprises a microsphere composition wherein the sterility of the microsphere is ensured by sterile filtration of the precursor organic solution and aqueous solutions. In another embodiment the auris-acceptable composition comprises a thermoreversible gel composition wherein the sterility of the gel composition is ensured by low temperature sterile filtration. In a further embodiment, the low temperature sterile filtration occurs at a temperature between 0 and 30° C., or between 0 and 20° C., or between 0 and 10° C., or between 10 and 20° C., or between 20 and 30° C. In another embodiment, is a process for the preparation of an auris-acceptable thermoreversible gel composition comprising: filtering the aqueous solution containing the thermoreversible gel components at low temperature through a sterilization filter; lyophilizing the sterile solution; and reconstituting the thermoreversible gel composition with sterile water prior to administration.

In certain embodiments, the active ingredients are dissolved in a suitable vehicle (e.g. a buffer) and sterilized separately (e.g. by heat treatment, filtration, gamma radiation). In some instances, the active ingredients are sterilized separately in a dry state. In some instances, the active ingredients are sterilized as a suspension or as a colloidal suspension. The remaining excipients (e.g., fluid gel components present in auris compositions) are sterilized in a separate step by a suitable method (e.g. filtration and/or irradiation of a cooled mixture of excipients); the two solutions that are separately sterilized are then mixed aseptically to provide a final auris composition. In some instances, the final aseptic mixing is performed just prior to administration of a composition described herein.

In some instances, conventionally used methods of sterilization (e.g., heat treatment (e.g., in an autoclave), gamma irradiation, filtration) lead to irreversible degradation of polymeric components (e.g., thermosetting, gelling or mucoadhesive polymer components) and/or the active agent in the composition. In some instances, sterilization of an auris composition by filtration through membranes (e.g., 0.2 μM membranes) is not possible if the composition comprises thixotropic polymers that gel during the process of filtration.

Accordingly, provided herein are methods for sterilization of auris compositions that prevent degradation of polymeric components (e.g., thermosetting and/or gelling and/or mucoadhesive polymer components) and/or the active agent during the process of sterilization. In some embodiments, degradation of the active agent (e.g., any therapeutic otic agent described herein) is reduced or eliminated through the use of specific pH ranges for buffer components and specific proportions of gelling agents in the compositions. In some embodiments, the choice of an appropriate gelling agent and/or thermosetting polymer allows for sterilization of compositions described herein by filtration. In some embodiments, the use of an appropriate thermosetting polymer and an appropriate copolymer (e.g., a gelling agent) in combination with a specific pH range for the composition allows for high temperature sterilization of compositions described with substantially no degradation of the therapeutic agent or the polymeric excipients. An advantage of the methods of sterilization provided herein is that, in certain instances, the compositions are subjected to terminal sterilization via autoclaving without any loss of the active agent and/or excipients and/or polymeric components during the sterilization step and are rendered substantially free of microbes and/or pyrogens.

Microorganisms

Provided herein are auris-acceptable compositions or devices that ameliorate or lessen otic disorders described herein. Further provided herein, are methods comprising the administration of said otic compositions. In some embodiments, the compositions or devices are substantially free of microorganisms. Acceptable sterility levels are based on applicable standards that define therapeutically acceptable otic compositions, including but not limited to United States Pharmacopeia Chapters <1111> et seq. For example, acceptable sterility levels include about 10 colony forming units (cfu) per gram of composition, about 50 cfu per gram of composition, about 100 cfu per gram of composition, about 500 cfu per gram of composition or about 1000 cfu per gram of composition. In some embodiments, acceptable sterility levels for compositions include less than 10 cfu/mL, less that 50 cfu/mL, less than 500 cfu/mL or less than 1000 cfu/mL microbial agents. In addition, acceptable sterility levels include the exclusion of specified objectionable microbiological agents. By way of example, specified objectionable microbiological agents include but are not limited to Escherichia coli (E. coli), Salmonella sp., Pseudomonas aeruginosa (P. aeruginosa) and/or other specific microbial agents.

Sterility of the auris-acceptable otic therapeutic agent composition is confirmed through a sterility assurance program in accordance with United States Pharmacopeia Chapters <61>, <62> and <71>. A key component of the sterility assurance quality control, quality assurance and validation process is the method of sterility testing. Sterility testing, by way of example only, is performed by two methods. The first is direct inoculation wherein a sample of the composition to be tested is added to growth medium and incubated for a period of time up to 21 days. Turbidity of the growth medium indicates contamination. Drawbacks to this method include the small sampling size of bulk materials that reduces sensitivity, and detection of microorganism growth based on a visual observation. An alternative method is membrane filtration sterility testing. In this method, a volume of product is passed through a small membrane filter paper. The filter paper is then placed into media to promote the growth of microorganisms. This method has the advantage of greater sensitivity as the entire bulk product is sampled. The commercially available Millipore Steritest sterility testing system is optionally used for determinations by membrane filtration sterility testing. For the filtration testing of creams or ointments Steritest filter system No. TLHVSL210 are used. For the filtration testing of emulsions or viscous products Steritest filter system No. TLAREM210 or TDAREM210 are used. For the filtration testing of pre-filled syringes Steritest filter system No. TTHASY210 are used. For the filtration testing of material dispensed as an aerosol or foam Steritest filter system No. TTHVA210 are used. For the filtration testing of soluble powders in ampoules or vials Steritest filter system No. TTHADA210 or TTHADV210 are used.

Testing for E. coli and Salmonella includes the use of lactose broths incubated at 30-35° C. for 24-72 hours, incubation in MacConkey and/or EMB agars for 18-24 hours, and/or the use of Rappaport medium. Testing for the detection of P. aeruginosa includes the use of NAC agar. United States Pharmacopeia Chapter <62> further enumerates testing procedures for specified objectionable microorganisms.

In certain embodiments, any controlled-release composition described herein has less than about 60 colony forming units (CFU), less than about 50 colony forming units, less than about 40 colony forming units, or less than about 30 colony forming units of microbial agents per gram of composition. In certain embodiments, the otic compositions described herein are formulated to be isotonic with the endolymph and/or the perilymph.

Endotoxins

Provided herein are otic compositions that ameliorate or lessen otic disorders described herein. Further provided herein, are methods comprising the administration of said otic compositions. In some embodiments, the compositions or devices are substantially free of endotoxins. An additional aspect of the sterilization process is the removal of by-products from the killing of microorganisms (hereinafter, “Product”). The process of depyrogenation removes pyrogens from the sample. Pyrogens are endotoxins or exotoxins that induce an immune response. An example of an endotoxin is the lipopolysaccharide (LPS) molecule found in the cell wall of gram-negative bacteria. While sterilization procedures such as autoclaving or treatment with ethylene oxide kill the bacteria, the LPS residue induces a proinflammatory immune response, such as septic shock. Because the molecular size of endotoxins can vary widely, the presence of endotoxins is expressed in “endotoxin units” (EU). One EU is equivalent to 100 picograms of E. coli LPS. Humans can develop a response to as little as 5 EU/kg of body weight. The sterility is expressed in any units as recognized in the art. In certain embodiments, otic compositions described herein contain lower endotoxin levels (e.g. <4 EU/kg of body weight of a subject) when compared to conventionally acceptable endotoxin levels (e.g., 5 EU/kg of body weight of a subject). In some embodiments, the auris-acceptable otic therapeutic agent composition has less than about 5 EU/kg of body weight of a subject. In other embodiments, the auris-acceptable otic therapeutic agent composition has less than about 4 EU/kg of body weight of a subject. In additional embodiments, the auris-acceptable otic therapeutic agent composition has less than about 3 EU/kg of body weight of a subject. In additional embodiments, the auris-acceptable otic therapeutic agent composition has less than about 2 EU/kg of body weight of a subject.

In some embodiments, the auris-acceptable otic therapeutic agent composition or device has less than about 5 EU/kg of composition. In other embodiments, the auris-acceptable otic therapeutic agent composition has less than about 4 EU/kg of composition. In additional embodiments, the auris-acceptable otic therapeutic agent composition has less than about 3 EU/kg of composition. In some embodiments, the auris-acceptable otic therapeutic agent composition has less than about 5 EU/kg Product. In other embodiments, the auris-acceptable otic therapeutic agent composition has less than about 1 EU/kg Product. In additional embodiments, the auris-acceptable otic therapeutic agent composition has less than about 0.2 EU/kg Product. In some embodiments, the auris-acceptable otic therapeutic agent composition has less than about 5 EU/g of unit or Product. In other embodiments, the auris-acceptable otic therapeutic agent composition has less than about 4 EU/g of unit or Product. In additional embodiments, the auris-acceptable otic therapeutic agent composition has less than about 3 EU/g of unit or Product. In some embodiments, the auris-acceptable otic therapeutic agent composition has less than about 5 EU/mg of unit or Product. In other embodiments, the auris-acceptable otic therapeutic agent composition has less than about 4 EU/mg of unit or Product. In additional embodiments, the auris-acceptable otic therapeutic agent composition has less than about 3 EU/mg of unit or Product. In certain embodiments, otic compositions described herein contain from about 1 to about 5 EU/mL of composition. In certain embodiments, otic compositions described herein contain from about 2 to about 5 EU/mL of composition, from about 3 to about 5 EU/mL of composition, or from about 4 to about 5 EU/mL of composition.

In certain embodiments, otic compositions or devices described herein contain lower endotoxin levels (e.g. <0.5 EU/mL of composition) when compared to conventionally acceptable endotoxin levels (e.g., 0.5 EU/mL of composition). In some embodiments, the auris-acceptable otic therapeutic agent composition or device has less than about 0.5 EU/mL of composition. In other embodiments, the auris-acceptable otic therapeutic agent composition has less than about 0.4 EU/mL of composition. In additional embodiments, the auris-acceptable otic therapeutic agent composition has less than about 0.2 EU/mL of composition.

Pyrogen detection, by way of example only, is performed by several methods. Suitable tests for sterility include tests described in United States Pharmacopoeia (USP)<71> Sterility Tests (23rd edition, 1995). The rabbit pyrogen test and the Limulus amebocyte lysate test are both specified in the United States Pharmacopeia Chapters <85> and <151> (USP23/NF 18, Biological Tests, The United States Pharmacopeial Convention, Rockville, Md., 1995). Alternative pyrogen assays have been developed based upon the monocyte activation-cytokine assay. Uniform cell lines suitable for quality control applications have been developed and have demonstrated the ability to detect pyrogenicity in samples that have passed the rabbit pyrogen test and the Limulus amebocyte lysate test (Taktak et al, J. Pharm. Pharmacol. (1990), 43:578-82). In an additional embodiment, the auris-acceptable otic therapeutic agent composition is subject to depyrogenation. In a further embodiment, the process for the manufacture of the auris-acceptable otic therapeutic agent composition comprises testing the composition for pyrogenicity. In certain embodiments, the compositions described herein are substantially free of pyrogens.

### pH and Practical Osmolarity

As used herein, “practical osmolarity/osmolality” or “deliverable osmolarity/osmolality” means the osmolarity/osmolality of a composition as determined by measuring the osmolarity/osmolality of the active agent and all excipients except the gelling and/or the thickening agent (e.g., polyoxyethylene-polyooxypropylene copolymers, carboxymethylcellulose or the like). The practical osmolarity of a composition described herein is measured by any suitable method, e.g., a freezing point depression method as described in Viegas et. al., Int. J. Pharm., 1998, 160, 157-162. In some instances, the practical osmolarity of a composition described herein is measured by vapor pressure osmometry (e.g., vapor pressure depression method) that allows for determination of the osmolarity of a composition at higher temperatures. In some instances, vapor pressure depression method allows for determination of the osmolarity of a composition comprising a gelling agent (e.g., a thermoreversible polymer) at a higher temperature wherein the gelling agent is in the form of a gel. The practical osmolality of an otic composition described herein is from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some embodiments, the compositions described herein have a practical osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L.

In some embodiments, the osmolarity at a target site of action (e.g., the perilymph) is about the same as the delivered osmolarity (i.e., osmolarity of materials that cross or penetrate the round window membrane) of any composition described herein. In some embodiments, the compositions described herein have a deliverable osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L.

The main cation present in the endolymph is potassium. In addition the endolymph has a high concentration of positively charged amino acids. The main cation present in the perilymph is sodium. In certain instances, the ionic composition of the endolymph and perilymph regulate the electrochemical impulses of hair cells. In certain instances, any change in the ionic balance of the endolymph or perilymph results in a loss of hearing due to changes in the conduction of electrochemical impulses along otic hair cells. In some embodiments, a composition disclosed herein does not disrupt the ionic balance of the perilymph. In some embodiments, a composition disclosed herein has an ionic balance that is the same as or substantially the same as the perilymph. In some embodiments, a composition disclosed herein does not disrupt the ionic balance of the endolymph. In some embodiments, a composition disclosed herein has an ionic balance that is the same as or substantially the same as the endolymph. In some embodiments, an otic composition described herein is formulated to provide an ionic balance that is compatible with inner ear fluids (e.g., endolymph and/or perilymph).

The endolymph and the perilymph have a pH that is close to the physiological pH of blood. The endolymph has a pH range of about 7.2-7.9; the perilymph has a pH range of about 7.2-7.4. The in situ pH of the proximal endolymph is about 7.4 while the pH of distal endolymph is about 7.9.

In some embodiments, the pH of a composition described herein is adjusted (e.g., by use of a buffer) to an endolymph-compatible pH range of about 5.5 to 9.0. In specific embodiments, the pH of a composition described herein is adjusted to a perilymph-suitable pH range of about 5.5 to about 9.0. In some embodiments, the pH of a composition described herein is adjusted to a perilymph-suitable range of about 5.5 to about 8.0, about 6 to about 8.0 or about 6.6 to about 8.0. In some embodiments, the pH of a composition described herein is adjusted to a perilymph-suitable pH range of about 7.0-7.6.

In some embodiments, useful compositions also include one or more pH adjusting agents or buffering agents. Suitable pH adjusting agents or buffers include, but are not limited to acetate, bicarbonate, ammonium chloride, citrate, phosphate, pharmaceutically acceptable salts thereof or combinations or mixtures thereof.

In one embodiment, when one or more buffers are utilized in the compositions of the present disclosure, they are combined (e.g., with a pharmaceutically acceptable vehicle) and are present in the final composition (e.g., in an amount ranging from about 0.1% to about 20%, from about 0.5% to about 10%). In certain embodiments of the present disclosure, the amount of buffer included in the gel compositions are an amount such that the pH of the gel composition does not interfere with the body's natural buffering system.

In one embodiment, diluents are also used to stabilize compounds because they can provide a more stable environment. Salts dissolved in buffered solutions (that also can provide pH control or maintenance) are utilized as diluents in the art, including, but not limited to a phosphate buffered saline solution.

In some embodiments, any gel composition described herein has a pH that allows for sterilization (e.g., by filtration or aseptic mixing or heat treatment and/or autoclaving (e.g., terminal sterilization)) of a gel composition without degradation of the pharmaceutical agent or the polymers comprising the gel. In order to reduce hydrolysis and/or degradation of the otic agent and/or the gel polymer during sterilization, the buffer pH is designed to maintain pH of the composition in the 7-8 range during the process of sterilization (e.g., high temperature autoclaving).

In specific embodiments, any gel composition described herein has a pH that allows for terminal sterilization (e.g., by heat treatment and/or autoclaving) of a gel composition without degradation of the pharmaceutical agent or the polymers comprising the gel. For example, in order to reduce hydrolysis and/or degradation of the otic agent and/or the gel polymer during autoclaving, the buffer pH is designed to maintain pH of the composition in the 7-8 range at elevated temperatures. Any appropriate buffer is used depending on the otic agent used in the composition. In some instances, since pKₐ of TRIS decreases as temperature increases at approximately −0.03/° C. and pKₐ of PBS increases as temperature increases at approximately 0.003/° C., autoclaving at 250° F. (121° C.) results in a significant downward pH shift (i.e. more acidic) in the TRIS buffer whereas a relatively much less upward pH shift in the PBS buffer and therefore much increased hydrolysis and/or degradation of an otic agent in TRIS than in PBS. Degradation of an otic agent is reduced by the use of an appropriate combination of a buffer and polymeric additives (e.g. P407, CMC) as described herein.

In some embodiments, a composition pH of between about 5.0 and about 9.0, between about 5.5 and about 8.5, between about 6.0 and about 7.6, between about 7 and about 7.8, between about 7.0 and about 7.6, between about 7.2 and 7.6, or between about 7.2 and about 7.4 is suitable for sterilization (e.g., by filtration or aseptic mixing or heat treatment and/or autoclaving (e.g., terminal sterilization)) of auris compositions described herein. In specific embodiments a composition pH of about 6.0, about 6.5, about 7.0, about 7.1, about 7.2, about 7.3, about 7.4, about 7.5, or about 7.6 is suitable for sterilization (e.g., by filtration or aseptic mixing or heat treatment and/or autoclaving (e.g., terminal sterilization)) of any composition described herein.

In some embodiments, the compositions have a pH as described herein, and include a thickening agent (e.g., a viscosity enhancing agent) such as, by way of non-limiting example, a cellulose based thickening agent described herein. In some instances, the addition of a secondary polymer (e.g., a thickening agent) and a pH of composition as described herein, allows for sterilization of a composition described herein without any substantial degradation of the otic agent and/or the polymer components in the otic composition. In some embodiments, the ratio of a thermoreversible poloxamer to a thickening agent in a composition that has a pH as described herein, is about 40:1, about 35:1, about 30:1, about 25:1, about 20:1, about 15:1 about 10:1, or about 5:1. For example, in certain embodiments, a sustained and/or extended release composition described herein comprises a combination of poloxamer 407 (pluronic F 127) and carboxymethylcellulose (CMC) in a ratio of about 40:1, about 35:1, about 30:1, about 25:1, about 20:1, about 15:1, about 10:1 or about 5:1.

In some embodiments, the amount of thermoreversible polymer in any composition described herein is about 10%, about 15%, about 20%, about 25%, about 30%, about 35% or about 40% of the total weight of the composition. In some embodiments, the amount of thermoreversible polymer in any composition described herein is about 10%, about 11%, about 12%, about 13%, about 14%, about 15%, about 16%, about 17%, about 18%, about 19%, about 20%, about 21%, about 22%, about 23%, about 24% or about 25% of the total weight of the composition. In some embodiments, the amount of thermoreversible polymer (e.g., pluronic F127) in any composition described herein is about 7.5% of the total weight of the composition. In some embodiments, the amount of thermoreversible polymer (e.g., pluronic F127) in any composition described herein is about 10% of the total weight of the composition. In some embodiments, the amount of thermoreversible polymer (e.g., pluronic F127) in any composition described herein is about 11% of the total weight of the composition. In some embodiments, the amount of thermoreversible polymer (e.g., pluronic F127) in any composition described herein is about 12% of the total weight of the composition. In some embodiments, the amount of thermoreversible polymer (e.g., pluronic F127) in any composition described herein is about 13% of the total weight of the composition. In some embodiments, the amount of thermoreversible polymer (e.g., pluronic F127) in any composition described herein is about 14% of the total weight of the composition. In some embodiments, the amount of thermoreversible polymer (e.g., pluronic F127) in any composition described herein is about 15% of the total weight of the composition. In some embodiments, the amount of thermoreversible polymer (e.g., pluronic F127) in any composition described herein is about 16% of the total weight of the composition. In some embodiments, the amount of thermoreversible polymer (e.g., pluronic F127) in any composition described herein is about 17% of the total weight of the composition. In some embodiments, the amount of thermoreversible polymer (e.g., pluronic F127) in any composition described herein is about 18% of the total weight of the composition. In some embodiments, the amount of thermoreversible polymer (e.g., pluronic F127) in any composition described herein is about 19% of the total weight of the composition. In some embodiments, the amount of thermoreversible polymer (e.g., pluronic F127) in any composition described herein is about 20% of the total weight of the composition. In some embodiments, the amount of thermoreversible polymer (e.g., pluronic F127) in any composition described herein is about 21% of the total weight of the composition. In some embodiments, the amount of thermoreversible polymer (e.g., pluronic F127) in any composition described herein is about 23% of the total weight of the composition. In some embodiments, the amount of thermoreversible polymer (e.g., pluronic F127) in any composition described herein is about 25% of the total weight of the composition.

In some embodiments, the amount of thickening agent (e.g., a gelling agent) in any composition described herein is about 1%, about 5%, about 10%, or about 15% of the total weight of the composition. In some embodiments, the amount of thickening agent (e.g., a gelling agent) in any composition described herein is about 0.5%, about 1%, about 1.5%, about 2%, about 2.5%, about 3%, about 3.5%, about 4%, about 4.5%, or about 5% of the total weight of the composition.

In some embodiments, the pharmaceutical compositions described herein are stable with respect to pH over a period of any of at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 1 month, at least about 2 months, at least about 3 months, at least about 4 months, at least about 5 months, or at least about 6 months. In other embodiments, the compositions described herein are stable with respect to pH over a period of at least about 1 week. Also described herein are compositions that are stable with respect to pH over a period of at least about 1 month.

Tonicity Agents

In general, the endolymph has a higher osmolality than the perilymph. For example, the endolymph has an osmolality of about 304 mOsm/kg H₂O while the perilymph has an osmolality of about 294 mOsm/kg H₂O. In certain embodiments, tonicity agents are added to the compositions described herein in an amount as to provide a practical osmolality of an otic composition of about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 350 mOsm/kg or from about 280 mOsm/kg to about 320 mOsm/kg. In some embodiments, the compositions described herein have a practical osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 280 mOsm/L to about 320 mOsm/L or about 250 mOsm/L to about 320 mOsm/L.

In some embodiments, the deliverable osmolarity of any composition described herein is designed to be isotonic with the targeted otic structure (e.g., endolymph, perilymph or the like). In specific embodiments, auris compositions described herein are formulated to provide a delivered perilymph-suitable osmolarity at the target site of action of about 250 to about 320 mOsm/L (osmolality of about 250 to about 320 mOsm/kg H₂O); and preferably about 270 to about 320 mOsm/L (osmolality of about 270 to about 320 mOsm/kg H₂O). In specific embodiments, the deliverable osmolarity/osmolality of the compositions (i.e., the osmolarity/osmolality of the composition in the absence of gelling or thickening agents (e.g., thermoreversible gel polymers)) is adjusted, for example, by the use of appropriate salt concentrations (e.g., concentration of potassium or sodium salts) or the use of tonicity agents that renders the compositions endolymph-compatible and/or perilymph-compatible (i.e. isotonic with the endolymph and/or perilymph) upon delivery at the target site. The osmolarity of a composition comprising a thermoreversible gel polymer is an unreliable measure due to the association of varying amounts of water with the monomeric units of the polymer. The practical osmolarity of a composition is a reliable measure and is measured by any suitable method (e.g., freezing point depression method, vapor depression method). In some instances, the compositions described herein provide a deliverable osmolarity (e.g., at a target site (e.g., perilymph)) that causes minimal disturbance to the environment of the inner ear and causes minimum discomfort (e.g., vertigo and/or nausea) to a mammal upon administration.

In some embodiments, any composition described herein is isotonic with the perilymph and/or endolymph. Isotonic compositions are provided by the addition of a tonicity agent. Suitable tonicity agents include, but are not limited to any pharmaceutically acceptable sugar, salt or any combinations or mixtures thereof, such as, but not limited to dextrose, glycerin, mannitol, sorbitol, sodium chloride, and other electrolytes.

Useful auris compositions include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some embodiments, the compositions described herein have a pH and/or practical osmolarity as described herein, and have a concentration of active pharmaceutical ingredient up to about 1 μM and about 10 μM, up to about 1 mM and about 100 mM, up to about 0.1 mM and about 100 mM, up to about 0.1 mM and about 100 nM. In some embodiments, the compositions described herein have a pH and/or practical osmolarity as described herein, and have a concentration of active pharmaceutical ingredient up to about 0.01%-about 20%, up to about 0.01%-about 10%., up to about 0.01%-about 7.5%, up to about 0.01%-6%, up to about 0.01-5%, up to about 0.1-about 10%, or up to about 0.1-about 6% of the active ingredient by weight of the composition. In some embodiments, the compositions described herein have a pH and/or practical osmolarity as described herein, and have a concentration of active pharmaceutical ingredient up to about 0.1 and about 70 mg, up to about 1 mg and about 70 mg/mL, up to about 1 mg and about 50 mg/mL, up to about 1 mg/mL and about 20 mg/mL, up to about 1 mg/mL to about 10 mg/mL, up to about 1 mg/mL to about 5 mg/mL, or up to about 0.5 mg/mL to about 5 mg/mL of the active agent by volume of the composition. In some embodiments, the compositions described herein have a pH and/or practical osmolarity as described herein, and have a concentration of active pharmaceutical ingredient up to about 1 μg/mL and about 500 μg/mL, up to about 1 μg/mL and about 250 μg/mL, up to about 1 μg and about 100 μg/mL, up to about 1 μg/mL and about 50 μg/mL, or up to about 1 μg/mL and about 20 μg/mL of the active agent by volume of the composition.

### Particle Size

Size reduction is used to increase surface area and/or modulate composition dissolution properties. It is also used to maintain a consistent average particle size distribution (PSD) (e.g., micrometer-sized particles, nanometer-sized particles or the like) for any composition described herein. In some embodiments, any composition described herein is multiparticulate (i.e., comprises a plurality of particle sizes (e.g., micronized particles, nano-sized particles, non-sized particles, colloidal particles)). In some embodiments, any composition described herein comprises one or more multiparticulate (e.g., micronized) therapeutic agents. Micronization is a process of reducing the average diameter of particles of a solid material. Micronized particles are from about micrometer-sized in diameter to about nanometer-sized in diameter. In some embodiments, the average diameter of particles in a micronized solid is from about 0.5 μm to about 500 μm. In some embodiments, the average diameter of particles in a micronized solid is from about 1 μm to about 200 μm. In some embodiments, the average diameter of particles in a micronized solid is from about 2 μm to about 100 μm. In some embodiments, the average diameter of particles in a micronized solid is from about 3 μm to about 50 μm. In some embodiments, a particulate micronized solid comprises particle sizes of less than about 5 microns, less than about 20 microns and/or less than about 100 microns. In some embodiments, the use of particulates (e.g., micronized particles) of an anti-apoptotic agent or pro-apoptotic agent allows for extended and/or sustained release of the anti-apoptotic agent or pro-apoptotic agent from any composition described herein compared to a composition comprising non-multiparticulate (e.g., non-micronized) anti-apoptotic agent or pro-apoptotic agent. In some instances, compositions containing multiparticulate (e.g. micronized) anti-apoptotic agent or pro-apoptotic agent are ejected from a 1 mL syringe adapted with a 27G needle without any plugging or clogging.

In some instances, any particle in any composition described herein is a coated particle (e.g., a coated micronized particle, nano-particle) and/or a microsphere and/or a liposomal particle. Particle size reduction techniques include, by way of example, grinding, milling (e.g., air-attrition milling (jet milling), ball milling), coacervation, complex coacervation, high pressure homogenization, spray drying and/or supercritical fluid crystallization. In some instances, particles are sized by mechanical impact (e.g., by hammer mills, ball mill and/or pin mills). In some instances, particles are sized via fluid energy (e.g., by spiral jet mills, loop jet mills, and/or fluidized bed jet mills). In some embodiments, compositions described herein comprise crystalline particles and/or isotropic particles. In some embodiments, compositions described herein comprise amorphous particles and/or anisotropic particles. In some embodiments, compositions described herein comprise therapeutic agent particles wherein the therapeutic agent is a neutral molecule, a free acid, a free base, or a salt, or a prodrug of a therapeutic agent, or any combination thereof.

In some embodiments, a composition described herein comprises one or more anti-apoptotic agent or pro-apoptotic agents wherein the anti-apoptotic agent or pro-apoptotic agent comprises nanoparticulates. In some embodiments, a composition described herein comprises anti-apoptotic agent or pro-apoptotic agent beads (e.g., tacrolimus beads) that are optionally coated with controlled-release excipients. In some embodiments, a composition described herein comprises an anti-apoptotic agent or pro-apoptotic agent that is granulated and/or reduced in size and coated with controlled-release excipients; the granulated coated anti-apoptotic agent or pro-apoptotic agent particulates are then optionally micronized and/or formulated in any of the compositions described herein.

In some instances, a combination of an anti-apoptotic agent or pro-apoptotic agent as a neutral molecule, a free acid, a free base and a salt of the anti-apoptotic agent or pro-apoptotic agent is used to prepare pulsed release otic agent compositions using the procedures described herein. In some compositions, a combination of a micronized anti-apoptotic agent or pro-apoptotic agent (and/or salt or prodrug thereof) and coated particles (e.g., nanoparticles, liposomes, microspheres) is used to prepare pulsed release otic agent compositions using any procedure described herein. Alternatively, a pulsed release profile is achieved by solubilizing up to 20% of the delivered dose of the anti-apoptotic agent or pro-apoptotic agent (e.g., micronized anti-apoptotic agent or pro-apoptotic agent, a neutral molecule, free base, free acid or salt or prodrug thereof; multiparticulate anti-apoptotic agent or pro-apoptotic agent, a neutral molecule, a free base, free acid or salt or prodrug thereof) with the aid of cyclodextrins, surfactants (e.g., poloxamers (407, 338, 188), tween (80, 60, 20, 81), PEG-hydrogenated castor oil, cosolvents like N-methyl-2-Pyrrolidone or the like and preparing pulsed release compositions using any procedure described herein.

In specific embodiments, any auris-compatible composition described herein comprises one or more micronized pharmaceutical agents (e.g., anti-apoptotic agent or pro-apoptotic agents). In some of such embodiments, a micronized pharmaceutical agent comprises micronized particles, coated (e.g., with an extended release coat) micronized particles, or a combination thereof. In some of such embodiments, a micronized pharmaceutical agent comprising micronized particles, coated micronized particles, or a combination thereof, comprises an anti-apoptotic agent or pro-apoptotic agent as a neutral molecule, a free acid, a free base, a salt, a prodrug or any combination thereof. In certain embodiments, a pharmaceutical composition described herein comprises an anti-apoptotic agent or pro-apoptotic agent as a micronized powder.

The multiparticulates and/or micronized anti-apoptotic agent or pro-apoptotic agents described herein are delivered to an auris structure (e.g., inner ear) by means of any type of matrix including solid, liquid or gel matrices. In some embodiments, the multiparticulates and/or micronized anti-apoptotic agent or pro-apoptotic agents described herein are delivered to an auris structure (e.g., inner ear) by means of any type of matrix including solid, liquid or gel matrices via intratympanic injection.

### Pharmaceutical Compositions

Provided herein are pharmaceutical compositions or devices that include at least one anti-apoptotic agent or pro-apoptotic agent and a pharmaceutically acceptable diluent(s), excipient(s), or carrier(s). In some embodiments, the pharmaceutical compositions include other medicinal or pharmaceutical agents, carriers, adjuvants, such as preserving, stabilizing, wetting or emulsifying agents, solution promoters, salts for regulating the osmotic pressure, and/or buffers. In other embodiments, the pharmaceutical compositions also contain other therapeutic substances.

Some pharmaceutical excipients, diluents or carriers are potentially ototoxic. For example, benzalkonium chloride, a common preservative, is ototoxic and therefore potentially harmful if introduced into the vestibular or cochlear structures. In formulating a controlled-release apoptosis modulating composition, it is advised to avoid or combine the appropriate excipients, diluents or carriers to lessen or eliminate potential ototoxic components from the composition, or to decrease the amount of such excipients, diluents or carriers. Optionally, a controlled-release apoptosis modulating composition includes otoprotective agents, such as antioxidants, alpha lipoic acid, calcium, fosfomycin or iron chelators, to counteract potential ototoxic effects that may arise from the use of specific therapeutic agents or excipients, diluents or carriers.

In some embodiments, the compositions or devices described herein include a dye to help enhance the visualization of the gel when applied. In some embodiments, dyes that are compatible with the auris-acceptable compositions or devices described herein include Evans blue (e.g., 0.5% of the total weight of an otic composition), Methylene blue (e.g., 1% of the total weight of an otic composition), Isosulfan blue (e.g., 1% of the total weight of an otic composition), Trypan blue (e.g., 0.15% of the total weight of an otic composition), and/or indocyanine green (e.g., 25 mg/vial). Other common dyes, e.g., FD&C red 40, FD&C red 3, FD&C yellow 5, FD&C yellow 6, FD&C blue 1, FD&C blue2, FD&C green 3, fluorescence dyes (e.g., Fluorescein isothiocyanate, rhodamine, Alexa Fluors, DyLight Fluors) and/or dyes that are visualizable in conjunction with non-invasive imaging techniques such as MRI, CAT scans, PET scans or the like. Gadolinium-based MRI dyes, iodine-base dyes, barium-based dyes or the like are also contemplated for use with any otic composition described herein. Other dyes that are compatible with any composition or composition described herein are listed in the Sigma-Aldrich catalog under dyes (that is included herein by reference for such disclosure).

Any pharmaceutical composition or device described herein is administered by locating the composition or device in contact with the crista fenestrae cochlea, the round window, the tympanic cavity, the tympanic membrane, the auris media or the auris externa.

In one specific embodiment of the auris-acceptable controlled-release anti-apoptotic agent or pro-apoptotic agent pharmaceutical compositions described herein, the anti-apoptotic agent or pro-apoptotic agent is provided in a gel matrix, also referred to herein as “auris acceptable gel compositions,” “auris interna-acceptable gel compositions,” “auris media-acceptable gel compositions,” “auris externa-acceptable gel compositions”, “auris gel compositions” or variations thereof. All of the components of the gel composition must be compatible with the targeted auris structure. Further, the gel compositions provide controlled-release of the anti-apoptotic agent or pro-apoptotic agent to the desired site within the targeted auris structure; in some embodiments, the gel composition also has an immediate or rapid release component for delivery of the anti-apoptotic agent or pro-apoptotic agent to the desired target site. In other embodiments, the gel composition has a sustained release component for delivery of the anti-apoptotic agent or pro-apoptotic agent. In some embodiments, the gel composition comprises a multiparticulate (e.g., micronized) anti-apoptotic agent or pro-apoptotic agent. In some embodiments, the auris gel compositions are biodegradable. In other embodiments, the auris gel compositions include a mucoadhesive excipient to allow adhesion to the external mucous layer of the round window membrane. In yet other embodiments, the auris gel compositions include a penetration enhancer excipient; in further embodiments, the auris gel composition contains a viscosity enhancing agent sufficient to provide a viscosity of between about 500 and 1,000,000 centipoise, between about 750 and 1,000,000 centipoise; between about 1000 and 1,000,000 centipoise; between about 1000 and 400,000 centipoise; between about 2000 and 100,000 centipoise; between about 3000 and 50,000 centipoise; between about 4000 and 25,000 centipoise; between about 5000 and 20,000 centipoise; or between about 6000 and 15,000 centipoise. In some embodiments, the auris gel composition contains a viscosity enhancing agent sufficient to provide a viscosity of between about 50,0000 and 1,000,000 centipoise.

In other embodiments, the auris interna pharmaceutical compositions described herein further provide an auris-acceptable hydrogel; in yet other embodiments, the auris pharmaceutical compositions provide an auris-acceptable microsphere or microparticle; in still other embodiments, the auris pharmaceutical compositions provide an auris-acceptable liposome. In some embodiments, the auris pharmaceutical compositions provide an auris-acceptable foam; in yet other embodiments, the auris pharmaceutical compositions provide an auris-acceptable paint; in still further embodiments, the auris pharmaceutical compositions provide an auris-acceptable in situ forming spongy material. In some embodiments, the auris pharmaceutical compositions provide an auris-acceptable solvent release gel. In some embodiments, the auris pharmaceutical compositions provide an actinic radiation curable gel. Further embodiments include a thermoreversible gel in the auris pharmaceutical composition, such that upon preparation of the gel at room temperature or below, the composition is a fluid, but upon application of the gel into or near the auris interna and/or auris media target site, including the tympanic cavity, round window membrane or the crista fenestrae cochleae, the auris-pharmaceutical composition stiffens or hardens into a gel-like substance.

In further or alternative embodiments, the auris gel compositions are capable of being administered on or near the round window membrane via intratympanic injection. In other embodiments, the auris gel compositions are administered on or near the round window or the crista fenestrae cochleae through entry via a post-auricular incision and surgical manipulation into or near the round window or the crista fenestrae cochleae area. Alternatively, the auris gel composition is applied via syringe and needle, wherein the needle is inserted through the tympanic membrane and guided to the area of the round window or crista fenestrae cochleae. The auris gel compositions are then deposited on or near the round window or crista fenestrae cochleae for localized treatment. In other embodiments, the auris gel compositions are applied via microcatheters implanted into the patient, and in yet further embodiments the compositions are administered via a pump device onto or near the round window membrane. In still further embodiments, the auris gel compositions are applied at or near the round window membrane via a microinjection device. In yet other embodiments, the auris gel compositions are applied in the tympanic cavity. In some embodiments, the auris gel compositions are applied on the tympanic membrane. In still other embodiments, the auris gel compositions are applied onto or in the auditory canal.

In further specific embodiments, any pharmaceutical composition or device described herein comprises a multiparticulate anti-apoptotic agent or pro-apoptotic agent in a liquid matrix (e.g., a liquid composition for intratympanic injection, or otic drops). In certain embodiments, any pharmaceutical composition described herein comprises a multiparticulate anti-apoptotic agent or pro-apoptotic agent in a solid matrix.

### Controlled-Release Compositions

In general, controlled-release drug compositions impart control over the release of drug with respect to site of release and time of release within the body. As discussed herein, controlled-release refers to immediate release, delayed release, sustained release, extended release, variable release, pulsatile release and bi-modal release. Many advantages are offered by controlled-release. First, controlled-release of a pharmaceutical agent allows less frequent dosing and thus minimizes repeated treatment. Second, controlled-release treatment results in more efficient drug utilization and less of the compound remains as a residue. Third, controlled-release offers the possibility of localized drug delivery by placement of a delivery device or composition at the site of disease. Still further, controlled-release offers the opportunity to administer and release two or more different drugs, each having a unique release profile, or to release the same drug at different rates or for different durations, by means of a single dosage unit.

Accordingly, one aspect of the embodiments disclosed herein is to provide a controlled-release apoptosis modulating auris-acceptable composition or. The controlled-release aspect of the compositions and/or compositions and/or devices disclosed herein is imparted through a variety of agents, including but not limited to excipients, agents or materials that are acceptable for use in the auris interna or other otic structure. By way of example only, such excipients, agents or materials include an auris-acceptable polymer, an auris-acceptable viscosity enhancing agent, an auris-acceptable gel, an auris-acceptable paint, an auris-acceptable foam, an auris-acceptable xerogel, an auris-acceptable microsphere or microparticle, an auris-acceptable hydrogel, an auris-acceptable in situ forming spongy material, an auris-acceptable actinic radiation curable gel, an auris-acceptable solvent release gel, an auris-acceptable liposome, an auris-acceptable nanocapsule or nanosphere, an auris-acceptable thermoreversible gel, or combinations thereof.

### Auris-Acceptable Gels

Gels, sometimes referred to as jellies, have been defined in various ways. For example, the United States Pharmacopoeia defines gels as semisolid systems consisting of either suspensions made up of small inorganic particles or large organic molecules interpenetrated by a liquid. Gels include a single-phase or a two-phase system. A single-phase gel consists of organic macromolecules distributed uniformly throughout a liquid in such a manner that no apparent boundaries exist between the dispersed macromolecules and the liquid. Some single-phase gels are prepared from synthetic macromolecules (e.g., carbomer) or from natural gums, (e.g., tragacanth). In some embodiments, single-phase gels are generally aqueous, but will also be made using alcohols and oils. Two-phase gels consist of a network of small discrete particles.

Gels can also be classified as being hydrophobic or hydrophilic. In certain embodiments, the base of a hydrophobic gel consists of liquid paraffin with polyethylene or fatty oils gelled with colloidal silica, or aluminum or zinc soaps. In contrast, the base of hydrophobic gels usually consists of water, glycerol, or propylene glycol gelled with a suitable gelling agent (e.g., tragacanth, starch, cellulose derivatives, carboxyvinylpolymers, and magnesium-aluminum silicates). In certain embodiments, the rheology of the compositions or devices disclosed herein is pseudo plastic, plastic, thixotropic, or dilatant.

In one embodiment the enhanced viscosity auris-acceptable composition described herein is not a liquid at room temperature. In certain embodiments, the enhanced viscosity composition is characterized by a phase transition between room temperature and body temperature (including an individual with a serious fever, e.g., up to about 42° C.). In some embodiments, the phase transition occurs at 1° C. below body temperature, at 2° C. below body temperature, at 3° C. below body temperature, at 4° C. below body temperature, at 6° C. below body temperature, at 8° C. below body temperature, or at 10° C. below body temperature. In some embodiments, the phase transition occurs at about 15° C. below body temperature, at about 20° C. below body temperature or at about 25° C. below body temperature. In specific embodiments, the gelation temperature (Tgel) of a composition described herein is about 20° C., about 25° C., or about 30° C. In certain embodiments, the gelation temperature (Tgel) of a composition described herein is about 35° C., or about 40° C. In one embodiment, administration of any composition described herein at about body temperature reduces or inhibits vertigo associated with intratympanic administration of otic compositions. Included within the definition of body temperature is the body temperature of a healthy individual, or an unhealthy individual, including an individual with a fever (up to ˜42° C.).). In some embodiments, the pharmaceutical compositions or devices described herein are liquids at about room temperature and are administered at or about room temperature, reducing or ameliorating side effects such as, for example, vertigo.

Polymers composed of polyoxypropylene and polyoxyethylene form thermoreversible gels when incorporated into aqueous solutions. These polymers have the ability to change from the liquid state to the gel state at temperatures close to body temperature, therefore allowing useful compositions that are applied to the targeted auris structure(s). The liquid state-to-gel state phase transition is dependent on the polymer concentration and the ingredients in the solution.

Poloxamer 407 (PF-127) is a nonionic surfactant composed of polyoxyethylene-polyoxypropylene copolymers. Other poloxamers include 188 (F-68 grade), 237 (F-87 grade), 338 (F-108 grade). Aqueous solutions of poloxamers are stable in the presence of acids, alkalis, and metal ions. PF-127 is a commercially available polyoxyethylene-polyoxypropylene triblock copolymer of general formula E106 P70 E106, with an average molar mass of 13,000. The polymer can be further purified by suitable methods that will enhance gelation properties of the polymer. It contains approximately 70% ethylene oxide, which accounts for its hydrophilicity. It is one of the series of poloxamer ABA block copolymers, whose members share the chemical formula shown below.

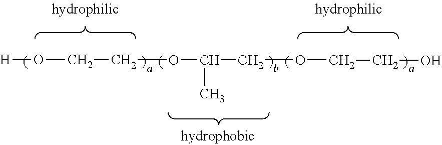

PF-127 is of particular interest since concentrated solutions (>20% w/w) of the copolymer are transformed from low viscosity transparent solutions to solid gels on heating to body temperature. This phenomenon, therefore, suggests that when placed in contact with the body, the gel preparation will form a semi-solid structure and a sustained release depot. Furthermore, PF-127 has good solubilizing capacity, low toxicity and is, therefore, considered a good medium for drug delivery systems.

In an alternative embodiment, the thermogel is a PEG-PLGA-PEG triblock copolymer (Jeong et al, Nature (1997), 388:860-2; Jeong et al, J. Control. Release (2000), 63:155-63; Jeong et al, Adv. Drug Delivery Rev. (2002), 54:37-51). The polymer exhibits sol-gel behavior over a concentration of about 5% w/w to about 40% w/w. Depending on the properties desired, the lactide/glycolide molar ratio in the PLGA copolymer ranges from about 1:1 to about 20:1. The resulting copolymers are soluble in water and form a free-flowing liquid at room temperature, but form a hydrogel at body temperature. A commercially available PEG-PLGA-PEG triblock copolymer is RESOMER RGP t50106 manufactured by Boehringer Ingelheim. This material is composed of a PGLA copolymer of 50:50 poly(DL-lactide-co-glycolide) and is 10% w/w of PEG and has a molecular weight of about 6000.

ReGel® is a tradename of MacroMed Incorporated for a class of low molecular weight, biodegradable block copolymers having reverse thermal gelation properties as described in U.S. Pat. Nos. 6,004,573, 6,117,949, 6,201,072, and 6,287,588. It also includes biodegradable polymeric drug carriers disclosed in pending U.S. patent application Ser. Nos. 09/906,041, 09/559,799 and 10/919,603. The biodegradable drug carrier comprises ABA-type or BAB-type triblock copolymers or mixtures thereof, wherein the A-blocks are relatively hydrophobic and comprise biodegradable polyesters or poly(orthoester)s, and the B-blocks are relatively hydrophilic and comprise polyethylene glycol (PEG), said copolymers having a hydrophobic content of between 50.1 to 83% by weight and a hydrophilic content of between 17 to 49.9% by weight, and an overall block copolymer molecular weight of between 2000 and 8000 Daltons. The drug carriers exhibit water solubility at temperatures below normal mammalian body temperatures and undergo reversible thermal gelation to then exist as a gel at temperatures equal to physiological mammalian body temperatures. The biodegradable, hydrophobic A polymer block comprises a polyester or poly(ortho ester), in that the polyester is synthesized from monomers selected from the group consisting of D,L-lactide, D-lactide, L-lactide, D,L-lactic acid, D-lactic acid, L-lactic acid, glycolide, glycolic acid, ε-caprolactone, ε-hydroxyhexanoic acid, γ-butyrolactone, γ-hydroxybutyric acid, δ-valerolactone, δ-hydroxyvaleric acid, hydroxybutyric acids, malic acid, and copolymers thereof and having an average molecular weight of between about 600 and 3000 Daltons. The hydrophilic B-block segment is preferably polyethylene glycol (PEG) having an average molecular weight of between about 500 and 2200 Daltons.

Additional biodegradable thermoplastic polyesters include AtriGel®(provided by Atrix Laboratories, Inc.) and/or those disclosed, e.g., in U.S. Pat. Nos. 5,324,519; 4,938,763; 5,702,716; 5,744,153; and 5,990,194; wherein the suitable biodegradable thermoplastic polyester is disclosed as a thermoplastic polymer. Examples of suitable biodegradable thermoplastic polyesters include polylactides, polyglycolides, polycaprolactones, copolymers thereof, terpolymers thereof, and any combinations thereof. In some such embodiments, the suitable biodegradable thermoplastic polyester is a polylactide, a polyglycolide, a copolymer thereof, a terpolymer thereof, or a combination thereof. In one embodiment, the biodegradable thermoplastic polyester is 50/50 poly(DL-lactide-co-glycolide) having a carboxy terminal group; is present in about 30 wt. % to about 40 wt. % of the composition; and has an average molecular weight of about 23,000 to about 45,000. Alternatively, in another embodiment, the biodegradable thermoplastic polyester is 75/25 poly (DL-lactide-co-glycolide) without a carboxy terminal group; is present in about 40 wt. % to about 50 wt. % of the composition; and has an average molecular weight of about 15,000 to about 24,000. In further or alternative embodiments, the terminal groups of the poly(DL-lactide-co-glycolide) are either hydroxyl, carboxyl, or ester depending upon the method of polymerization. Polycondensation of lactic or glycolic acid provides a polymer with terminal hydroxyl and carboxyl groups. Ring-opening polymerization of the cyclic lactide or glycolide monomers with water, lactic acid, or glycolic acid provides polymers with the same terminal groups. However, ring-opening of the cyclic monomers with a monofunctional alcohol such as methanol, ethanol, or 1-dodecanol provides a polymer with one hydroxyl group and one ester terminal groups. Ring-opening polymerization of the cyclic monomers with a diol such as 1,6-hexanediol or polyethylene glycol provides a polymer with only hydroxyl terminal groups.

Since the polymer systems of thermoreversible gels dissolve more completely at reduced temperatures, methods of solubilization include adding the required amount of polymer to the amount of water to be used at reduced temperatures. Generally after wetting the polymer by shaking, the mixture is capped and placed in a cold chamber or in a thermostatic container at about 0-10° C. in order to dissolve the polymer. The mixture is stirred or shaken to bring about a more rapid dissolution of the thermoreversible gel polymer. The anti-apoptotic agent or pro-apoptotic agent and various additives such as buffers, salts, and preservatives are subsequently added and dissolved. In some instances the anti-apoptotic agent or pro-apoptotic agent and/or other pharmaceutically active agent is suspended if it is insoluble in water. The pH is modulated by the addition of appropriate buffering agents. round window membrane mucoadhesive characteristics are optionally imparted to a thermoreversible gel by incorporation of round window membrane mucoadhesive carbomers, such as Carbopol® 934P, to the composition (Majithiya et al, AAPS PharmSciTech (2006), 7(3), p. E1; EP0551626, both of that is incorporated herein by reference for such disclosure).

In one embodiment are auris-acceptable pharmaceutical gel compositions that do not require the use of an added viscosity enhancing agent. Such gel compositions incorporate at least one pharmaceutically acceptable buffer. In one aspect, is a gel composition comprising an anti-apoptotic agent or pro-apoptotic agent and a pharmaceutically acceptable buffer. In another embodiment, the pharmaceutically acceptable excipient or carrier is a gelling agent.

In other embodiments, useful anti-apoptotic agent or pro-apoptotic agent auris-acceptable pharmaceutical compositions also include one or more pH adjusting agents or buffering agents to provide an endolymph or perilymph suitable pH. Suitable pH adjusting agents or buffers include, but are not limited to acetate, bicarbonate, ammonium chloride, citrate, phosphate, pharmaceutically acceptable salts thereof or combinations or mixtures thereof. Such pH adjusting agents and buffers are included in an amount required to maintain pH of the composition between a pH of about 5 and about 9, in one embodiment a pH between about 6.5 to about 7.5, and in yet another embodiment at a pH of about 6.5, 6.6, 6.7, 6.8, 6.9, 7.0, 7.1, 7.2, 7.3, 7.4, 7.5. In one embodiment, when one or more buffers are utilized in the compositions of the present disclosure, they are combined, e.g., with a pharmaceutically acceptable vehicle and are present in the final composition, e.g., in an amount ranging from about 0.1% to about 20%, from about 0.5% to about 10%. In certain embodiments of the present disclosure, the amount of buffer included in the gel compositions is an amount such that the pH of the gel composition does not interfere with the natural buffering system of the auris media or auris interna, or does not interfere with the natural pH of the endolymph or perilymph: depending on where in the cochlea the anti-apoptotic agent or pro-apoptotic agent composition is targeted. In some embodiments, from about 10 μM to about 200 mM concentration of a buffer is present in the gel composition. In certain embodiments, from about a 5 mM to about a 200 mM concentration of a buffer is present. In certain embodiments, from about a 20 mM to about a 100 mM concentration of a buffer is present. In one embodiment is a buffer such as acetate or citrate at slightly acidic pH. In one embodiment the buffer is a sodium acetate buffer having a pH of about 4.5 to about 6.5. In one embodiment the buffer is a sodium citrate buffer having a pH of about 5.0 to about 8.0, or about 5.5 to about 7.0.

In an alternative embodiment, the buffer used is tris(hydroxymethyl)aminomethane, bicarbonate, carbonate or phosphate at slightly basic pH. In one embodiment, the buffer is a sodium bicarbonate buffer having a pH of about 6.5 to about 8.5, or about 7.0 to about 8.0. In another embodiment the buffer is a sodium phosphate dibasic buffer having a pH of about 6.0 to about 9.0.

Also described herein, are controlled-release compositions or devices comprising an anti-apoptotic agent or pro-apoptotic agent and a viscosity enhancing agent. Suitable viscosity-enhancing agents include by way of example only, gelling agents and suspending agents. In one embodiment, the enhanced viscosity composition does not include a buffer. In other embodiments, the enhanced viscosity composition includes a pharmaceutically acceptable buffer. Sodium chloride or other tonicity agents are optionally used to adjust tonicity, if necessary.

By way of example only, the auris-acceptable viscosity agent includes hydroxypropyl methylcellulose, hydroxyethyl cellulose, polyvinylpyrrolidone, carboxymethyl cellulose, polyvinyl alcohol, sodium chondroitin sulfate, sodium hyaluronate. Other viscosity enhancing agents compatible with the targeted auris structure include, but are not limited to, acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose) or combinations thereof. In specific embodiments, the viscosity-enhancing excipient is a combination of MCC and CMC. In another embodiment, the viscosity-enhancing agent is a combination of carboxymethylated chitosan, or chitin, and alginate. The combination of chitin and alginate with the anti-apoptotic agent or pro-apoptotic agents disclosed herein acts as a controlled-release composition, restricting the diffusion of the anti-apoptotic agent or pro-apoptotic agents from the composition. Moreover, the combination of carboxymethylated chitosan and alginate is optionally used to assist in increasing the permeability of the anti-apoptotic agent or pro-apoptotic agents through the round window membrane.

In some embodiments, is an enhanced viscosity composition, comprising from about 0.1 mM and about 100 mM of an anti-apoptotic agent or pro-apoptotic agent, a pharmaceutically acceptable viscosity agent, and water for injection, the concentration of the viscosity agent in the water being sufficient to provide a enhanced viscosity composition with a final viscosity from about 100 to about 100,000 cP. In certain embodiments, the viscosity of the gel is in the range from about 100 to about 50,000 cP, about 100 cP to about 1,000 cP, about 500 cP to about 1500 cP, about 1000 cP to about 3000 cP, about 2000 cP to about 8,000 cP, about 4,000 cP to about 50,000 cP, about 10,000 cP to about 500,000 cP, about 15,000 cP to about 1,000,000 cP. In other embodiments, when an even more viscous medium is desired, the biocompatible gel comprises at least about 35%, at least about 45%, at least about 55%, at least about 65%, at least about 70%, at least about 75%, or even at least about 80% or so by weight of the anti-apoptotic agent or pro-apoptotic agent. In highly concentrated samples, the biocompatible enhanced viscosity composition comprises at least about 25%, at least about 35%, at least about 45%, at least about 55%, at least about 65%, at least about 75%, at least about 85%, at least about 90% or at least about 95% or more by weight of the anti-apoptotic agent or pro-apoptotic agent.

In some embodiments, the viscosity of the gel compositions presented herein are measured by any means described. For example, in some embodiments, an LVDV-II+CP Cone Plate Viscometer and a Cone Spindle CPE-40 is used to calculate the viscosity of the gel composition described herein. In other embodiments, a Brookfield (spindle and cup) viscometer is used to calculate the viscosity of the gel composition described herein. In some embodiments, the viscosity ranges referred to herein are measured at room temperature. In other embodiments, the viscosity ranges referred to herein are measured at body temperature (e.g., at the average body temperature of a healthy human).

In one embodiment, the pharmaceutically acceptable enhanced viscosity auris-acceptable composition comprises at least one anti-apoptotic agent or pro-apoptotic agent and at least one gelling agent. Suitable gelling agents for use in preparation of the gel composition include, but are not limited to, celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum and any combinations or mixtures thereof. In some other embodiments, hydroxypropylmethylcellulose (Methocel®) is utilized as the gelling agent. In certain embodiments, the viscosity enhancing agents described herein are also utilized as the gelling agent for the gel compositions presented herein.

In some embodiments, the otic therapeutic agents disclosed herein are dispensed as an auris-acceptable paint. As used herein, paints (also known as film formers) are solutions comprised of a solvent, a monomer or polymer, an active agent, and optionally one or more pharmaceutically-acceptable excipients. After application to a tissue, the solvent evaporates leaving behind a thin coating comprised of the monomers or polymers, and the active agent. The coating protects active agents and maintains them in an immobilized state at the site of application. This decreases the amount of active agent that may be lost and correspondingly increases the amount delivered to the subject. By way of non-limiting example, paints include collodions (e.g. Flexible Collodion, USP), and solutions comprising saccharide siloxane copolymers and a cross-linking agent. Collodions are ethyl ether/ethanol solutions containing pyroxylin (a nitrocellulose). After application, the ethyl ether/ethanol solution evaporates leaving behind a thin film of pyroxylin. In solutions comprising saccharide siloxane copolymers, the saccharide siloxane copolymers form the coating after evaporation of the solvent initiates the cross-linking of the saccharide siloxane copolymers. For additional disclosures regarding paints, see Remington: The Science and Practice of Pharmacy that is hereby incorporated with respect to this subject matter. The paints contemplated for use herein, are flexible such that they do not interfere with the propagation of pressure waves through the ear. Further, the paints may be applied as a liquid (i.e. solution, suspension, or emulsion), a semisolid (i.e. a gel, foam, paste, or jelly) or an aerosol.

In some embodiments, an otic therapeutic agent disclosed herein is dispensed as a controlled-release foam. Examples of suitable foamable carriers for use in the compositions disclosed herein include, but are not limited to, alginate and derivatives thereof, carboxymethylcellulose and derivatives thereof, collagen, polysaccharides, including, for example, dextran, dextran derivatives, pectin, starch, modified starches such as starches having additional carboxyl and/or carboxamide groups and/or having hydrophilic side-chains, cellulose and derivatives thereof, agar and derivatives thereof, such as agar stabilized with polyacrylamide, polyethylene oxides, glycol methacrylates, gelatin, gums such as xanthum, guar, karaya, gellan, arabic, tragacanth and locust bean gum, or combinations thereof. Also suitable are the salts of the aforementioned carriers, for example, sodium alginate. The composition optionally further comprises a foaming agent, which promotes the formation of the foam, including a surfactant or external propellant. Examples of suitable foaming agents include cetrimide, lecithin, soaps, silicones and the like. Commercially available surfactants such as Tween® are also suitable.

In some embodiments, other gel compositions are useful depending upon the particular anti-apoptotic agent or pro-apoptotic agent, other pharmaceutical agent or excipients/additives used, and as such are considered to fall within the scope of the present disclosure. For example, other commercially-available glycerin-based gels, glycerin-derived compounds, conjugated, or crosslinked gels, matrices, hydrogels, and polymers, as well as gelatins and their derivatives, alginates, and alginate-based gels, and even various native and synthetic hydrogel and hydrogel-derived compounds are all expected to be useful in the anti-apoptotic agent or pro-apoptotic agent compositions described herein. In some embodiments, auris-acceptable gels include, but are not limited to, alginate hydrogels SAF®-Gel (ConvaTec, Princeton, N.J.), Duoderm® Hydroactive Gel (ConvaTec), Nu-gel® (Johnson & Johnson Medical, Arlington, Tex.); Carrasyn® (V) Acemannan Hydrogel (Carrington Laboratories, Inc., Irving, Tex.); glycerin gels Elta® Hydrogel (Swiss-American Products, Inc., Dallas, Tex.) and K-Y® Sterile (Johnson & Johnson). In further embodiments, biodegradable biocompatible gels also represent compounds present in auris-acceptable compositions disclosed and described herein.

In some compositions developed for administration to a mammal, and for compositions formulated for human administration, the auris-acceptable gel comprises substantially all of the weight of the composition. In other embodiments, the auris-acceptable gel comprises as much as about 98% or about 99% of the composition by weight. This is desirous when a substantially non-fluid, or substantially viscous composition is needed. In a further embodiment, when slightly less viscous, or slightly more fluid auris-acceptable pharmaceutical gel compositions are desired, the biocompatible gel portion of the composition comprises at least about 50% by weight, at least about 60% by weight, at least about 70% by weight, or even at least about 80% or 90% by weight of the compound. All intermediate integers within these ranges are contemplated to fall within the scope of this disclosure, and in some alternative embodiments, even more fluid (and consequently less viscous) auris-acceptable gel compositions are formulated, such as for example, those in that the gel or matrix component of the mixture comprises not more than about 50% by weight, not more than about 40% by weight, not more than about 30% by weight, or even those than comprise not more than about 15% or about 20% by weight of the composition.

Auris-Acceptable Suspending Agents

In one embodiment, at least one anti-apoptotic agent or pro-apoptotic agent is included in a pharmaceutically acceptable enhanced viscosity composition wherein the composition further comprises at least one suspending agent, wherein the suspending agent assists in imparting controlled-release characteristics to the composition. In some embodiments, suspending agents also serve to increase the viscosity of the auris-acceptable apoptosis modulating compositions and compositions.

Suspending agents include, by way of example only, compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), sodium carboxymethylcellulose, methylcellulose, hydroxypropylmethylcellulose (hypromellose), hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like. In some embodiments, useful aqueous suspensions also contain one or more polymers as suspending agents. Useful polymers include water-soluble polymers such as cellulosic polymers, e.g., hydroxypropyl methylcellulose, and water-insoluble polymers such as cross-linked carboxyl-containing polymers.

In one embodiment, the present disclosure provides auris-acceptable gel compositions comprising a therapeutically effective amount of an anti-apoptotic agent or pro-apoptotic agent in a hydroxyethyl cellulose gel. Hydroxyethyl cellulose (HEC) is obtained as a dry powder that is reconstituted in water or an aqueous buffer solution to give the desired viscosity (generally about 200 cps to about 30,000 cps, corresponding to about 0.2 to about 10% HEC). In one embodiment the concentration of HEC is between about 1% and about 15%, about 1% and about 2%, or about 1.5% to about 2%.

In other embodiments, the auris-acceptable compositions, including gel compositions and viscosity-enhanced compositions, further include excipients, other medicinal or pharmaceutical agents, carriers, adjuvants, such as preserving, stabilizing, wetting or emulsifying agents, solution promoters, salts, solubilizers, an antifoaming agent, an antioxidant, a dispersing agent, a wetting agent, a surfactant, or combinations thereof.

Auris-Acceptable Actinic Radiation Curable Gel

In other embodiments, the gel is an actinic radiation curable gel, such that following administration to or near the targeted auris structure, use of actinic radiation (or light, including UV light, visible light, or infrared light) the desired gel properties are formed. By way of example only, fiber optics are used to provide the actinic radiation so as to form the desired gel properties. In some embodiments, the fiber optics and the gel administration device form a single unit. In other embodiments, the fiber optics and the gel administration device are provided separately.

Auris-Acceptable Solvent Release Gel

In some embodiments, the gel is a solvent release gel such that the desired gel properties are formed after administration to or near the targeted auris structure, which is, as the solvent in the injected gel composition diffuses out the gel, a gel having the desired gel properties is formed. For example, a composition that comprises sucrose acetate isobutyrate, a pharmaceutically acceptable solvent, one or more additives, and the anti-apoptotic agent or pro-apoptotic agent is administered at or near the round window membrane: diffusion of the solvent out of the injected composition provides a depot having the desired gel properties. For example, use of a water soluble solvent provides a high viscosity depot when the solvent diffuses rapidly out of the injected composition. On the other hand, use of a hydrophobic solvent (e.g., benzyl benzoate) provides a less viscous depot. One example of an auris-acceptable solvent release gel composition is the SABER® Delivery System marketed by DURECT Corporation.

Auris-Acceptable In Situ Forming Spongy Material

Also contemplated within the scope of the embodiments is the use of a spongy material, formed in situ in the auris interna or auris media. In some embodiments, the spongy material is formed from hyaluronic acid or its derivatives. The spongy material is impregnated with a desired anti-apoptotic agent or pro-apoptotic agent and placed within the auris media so as to provide controlled-release of the anti-apoptotic agent or pro-apoptotic agent within the auris media, or in contact with the round window membrane so as to provide controlled-release of the anti-apoptotic agent or pro-apoptotic agent into the auris interna. In some embodiments, the spongy material is biodegradable.

Round Window Membrane Mucoadhesives

Also contemplated within the scope of the embodiments is the addition of a round window membrane mucoadhesive with the anti-apoptotic agent or pro-apoptotic agent compositions and compositions and devices disclosed herein. The term ‘mucoadhesion’ is used for materials that bind to the mucin layer of a biological membrane, such as the external membrane of the 3-layered round window membrane. To serve as round window membrane mucoadhesive polymers, the polymers possess some general physiochemical features such as predominantly anionic hydrophilicity with numerous hydrogen bond forming groups, suitable surface property for wetting mucus/mucosal tissue surfaces or sufficient flexibility to penetrate the mucus network.

Round window membrane mucoadhesive agents that are used with the auris-acceptable compositions include, but are not limited to, at least one soluble polyvinylpyrrolidone polymer (PVP); a water-swellable, but water-insoluble, fibrous, cross-linked carboxy-functional polymer; a crosslinked poly(acrylic acid) (e.g. Carbopol® 947P); a carbomer homopolymer; a carbomer copolymer; a hydrophilic polysaccharide gum, maltodextrin, a cross-linked alignate gum gel, a water-dispersible polycarboxylated vinyl polymer, at least two particulate components selected from the group consisting of titanium dioxide, silicon dioxide, and clay, or a mixture thereof. The round window membrane mucoadhesive agent is optionally used in combination with an auris-acceptable viscosity increasing excipient, or used alone to increase the interaction of the composition with the mucosal layer target otic component. In one non-limiting example, the mucoadhesive agent is maltodextrin and/or an alginate gum. When used, the round window membrane mucoadhesive character imparted to the composition is at a level that is sufficient to deliver an effective amount of the anti-apoptotic agent or pro-apoptotic agent composition to, for example, the mucosal layer of round window membrane or the crista fenestrae cochleae in an amount that coats the mucosal membrane, and thereafter deliver the composition to the affected areas, including by way of example only, the vestibular and/or cochlear structures of the auris interna. When used, the mucoadhesive characteristics of the compositions provided herein are determined, and using this information (along with the other teachings provided herein), the appropriate amounts are determined. One method for determining sufficient mucoadhesiveness includes monitoring changes in the interaction of the composition with a mucosal layer, including but not limited to measuring changes in residence or retention time of the composition in the absence and presence of the mucoadhesive excipient.

Mucoadhesive agents have been described, for example, in U.S. Pat. Nos. 6,638,521, 6,562,363, 6,509,028, 6,348,502, 6,319,513, 6,306,789, 5,814,330, and 4,900,552, each of that is hereby incorporated by reference for such disclosure.

In another non-limiting example, a mucoadhesive agent is, for example, at least two particulate components selected from titanium dioxide, silicon dioxide, and clay, wherein the composition is not further diluted with any liquid prior to administration and the level of silicon dioxide, if present, is from about 3% to about 15%, by weight of the composition. Silicon dioxide, if present, includes fumed silicon dioxide, precipitated silicon dioxide, coacervated silicon dioxide, gel silicon dioxide, and mixtures thereof. Clay, if present, includes kaolin minerals, serpentine minerals, smectites, illite or a mixture thereof. For example, clay includes laponite, bentonite, hectorite, saponite, montmorillonites or a mixture thereof.

In one non-limiting example, the round window membrane mucoadhesive agent is maltodextrin. Maltodextrin is a carbohydrate produced by the hydrolysis of starch that is optionally derived from corn, potato, wheat or other plant products. Maltodextrin is optionally used either alone or in combination with other round window membrane mucoadhesive agents to impart mucoadhesive characteristics on the compositions disclosed herein. In one embodiment, a combination of maltodextrin and a carbopol polymer are used to increase the round window membrane mucoadhesive characteristics of the compositions or devices disclosed herein.

In another embodiment, the round window membrane mucoadhesive agent is an alkyl-glycoside and/or a saccharide alkyl ester. As used herein, an “alkyl-glycoside” means a compound comprising any hydrophilic saccharide (e.g. sucrose, maltose, or glucose) linked to a hydrophobic alkyl. In some embodiments, the round window membrane mucoadhesive agent is an alkyl-glycoside wherein the alkyl-glycoside comprises a sugar linked to a hydrophobic alkyl (e.g., an alkyl comprising about 6 to about 25 carbon atoms) by an amide linkage, an amine linkage, a carbamate linkage, an ether linkage, a thioether linkage, an ester linkage, a thioester linkage, a glycosidic linkage, a thioglycosidic linkage, and/or a ureide linkage. In some embodiments, the round window membrane mucoadhesive agent is a hexyl-, heptyl-, octyl-, nonyl-, decyl-, undecyl-, dodecyl-, tridecyl-, tetradecyl, pentadecyl-, hexadecyl-, heptadecyl-, and octadecyl α- or β-D-maltoside; hexyl-, heptyl-, octyl-, nonyl-, decyl-, undecyl-, dodecyl-, tridecyl-, tetradecyl, pentadecyl-, hexadecyl-, heptadecyl-, and octadecyl α- or β-D-glucoside; hexyl-, heptyl-, octyl-, nonyl-, decyl-, undecyl-, dodecyl-, tridecyl-, tetradecyl, pentadecyl-, hexadecyl-, heptadecyl-, and octadecyl α- or β-D-sucroside; hexyl-, heptyl-, octyl-, dodecyl-, tridecyl-, and tetradecyl-β-D-thiomaltoside; heptyl- or octyl-1-thio-α- or β-D-glucopyranoside; alkyl thiosucroses; alkyl maltotriosides; long chain aliphatic carbonic acid amides of sucrose β-amino-alkyl ethers; derivatives of palatinose or isomaltamine linked by an amide linkage to an alkyl chain and derivatives of isomaltamine linked by urea to an alkyl chain; long chain aliphatic carbonic acid ureides of sucrose β-amino-alkyl ethers and long chain aliphatic carbonic acid amides of sucrose β-amino-alkyl ethers. In some embodiments, the round window membrane mucoadhesive agent is an alkyl-glycoside wherein the alkyl glycoside is maltose, sucrose, glucose, or a combination thereof linked by a glycosidic linkage to an alkyl chain of 9-16 carbon atoms (e.g., nonyl-, decyl-, dodecyl- and tetradecyl sucroside; nonyl-, decyl-, dodecyl- and tetradecyl glucoside; and nonyl-, decyl-, dodecyl- and tetradecyl maltoside). In some embodiments, the round window membrane mucoadhesive agent is an alkyl-glycoside wherein the alkyl glycoside is dodecylmaltoside, tridecylmaltoside, and tetradecylmaltoside.

In some embodiments, the round window membrane mucoadhesive agent is an alkyl-glycoside wherein the alkyl-glycoside is a disaccharide with at least one glucose. In some embodiments, the auris acceptable penetration enhancer is a surfactant comprising α-D-glucopyranosyl-β-glycopyranoside, n-Dodecyl-4-O-α-D-glucopyranosyl-β-glycopyranoside, and/or n-tetradecyl-4-O-α-D-glucopyranosyl-β-glycopyranoside. In some embodiments, the round window membrane mucoadhesive agent is an alkyl-glycoside wherein the alkyl-glycoside has a critical miscelle concentration (CMC) of less than about 1 mM in pure water or in aqueous solutions. In some embodiments, the round window membrane mucoadhesive agent is an alkyl-glycoside wherein an oxygen atom within the alkyl-glycoside is substituted with a sulfur atom. In some embodiments, the round window membrane mucoadhesive agent is an alkyl-glycoside wherein the alkylglycoside is the β anomer. In some embodiments, the round window membrane mucoadhesive agent is an alkyl-glycoside wherein the alkylglycoside comprises 90%, 91%, 92%, 93%, 94%, 95%, 96%, 97%, 98%, 99%, 99.1%, 99.5%, or 99.9% of the β anomer.

Auris-Acceptable Controlled-Release Particles

anti-apoptotic agent or pro-apoptotic agents and/or other pharmaceutical agents disclosed herein are optionally incorporated within controlled-release particles, lipid complexes, liposomes, nanoparticles, microparticles, microspheres, coacervates, nanocapsules or other agents that enhance or facilitate the localized delivery of the anti-apoptotic agent or pro-apoptotic agent. In some embodiments, a single enhanced viscosity composition is used, in that at least one anti-apoptotic agent or pro-apoptotic agent is present, while in other embodiments, a pharmaceutical composition that comprises a mixture of two or more distinct enhanced viscosity compositions is used, in that at least one anti-apoptotic agent or pro-apoptotic agent is present. In some embodiments, combinations of sols, gels and/or biocompatible matrices is also employed to provide desirable characteristics of the controlled-release apoptosis modulating compositions or compositions. In certain embodiments, the controlled-release apoptosis modulating compositions or compositions are cross-linked by one or more agents to alter or improve the properties of the composition.

Examples of microspheres relevant to the pharmaceutical compositions disclosed herein include: Luzzi, L. A., J. Pharm. Psy. 59:1367 (1970); U.S. Pat. No. 4,530,840; Lewis, D. H., “Controlled-release of Bioactive Agents from Lactides/Glycolide Polymers” in Biodegradable Polymers as Drug Delivery Systems, Chasin, M. and Langer, R., eds., Marcel Decker (1990); U.S. Pat. No. 4,675,189; Beck et al., “Poly(lactic acid) and Poly(lactic acid-co-glycolic acid) Contraceptive Delivery Systems,” in Long Acting Steroid Contraception, Mishell, D. R., ed., Raven Press (1983); U.S. Pat. No. 4,758,435; U.S. Pat. No. 3,773,919; U.S. Pat. No. 4,474,572. Examples of protein therapeutics formulated as microspheres include: U.S. Pat. No. 6,458,387; U.S. Pat. No. 6,268,053; U.S. Pat. No. 6,090,925; U.S. Pat. No. 5,981,719; and U.S. Pat. No. 5,578,709, and are herein incorporated by reference for such disclosure.

Microspheres usually have a spherical shape, although irregularly-shaped microparticles are possible. Microspheres may vary in size, ranging from submicron to 1000 micron diameters. Microspheres suitable for use with the auris-acceptable compositions disclosed herein are submicron to 250 micron diameter microspheres, allowing administration by injection with a standard gauge needle. The auris-acceptable microspheres are prepared by any method that produces microspheres in a size range acceptable for use in an injectable composition. Injection is optionally accomplished with standard gauge needles used for administering liquid compositions.

Suitable examples of polymeric matrix materials for use in the auris-acceptable controlled-release particles herein include poly(glycolic acid), poly-d,l-lactic acid, poly-l-lactic acid, copolymers of the foregoing, poly(aliphatic carboxylic acids), copolyoxalates, polycaprolactone, polydioxonene, poly(orthocarbonates), poly(acetals), poly(lactic acid-caprolactone), polyorthoesters, poly(glycolic acid-caprolactone), polydioxonene, polyanhydrides, polyphosphazines, and natural polymers including albumin, casein, and some waxes, such as, glycerol mono- and distearate, and the like. Various commercially available poly (lactide-co-glycolide) materials (PLGA) are optionally used in the method disclosed herein. For example, poly (d,l-lactic-co-glycolic acid) is commercially available from Boehringer-Ingelheim as RESOMER RG 503H. This product has a mole percent composition of 50% lactide and 50% glycolide. These copolymers are available in a wide range of molecular weights and ratios of lactic acid to glycolic acid. One embodiment includes the use of the polymer poly(d,l-lactide-co-glycolide). The molar ratio of lactide to glycolide in such a copolymer includes the range of from about 95:5 to about 50:50.

The molecular weight of the polymeric matrix material is of some importance. The molecular weight should be high enough so that it forms satisfactory polymer coatings, i.e., the polymer should be a good film former. Usually, a satisfactory molecular weight is in the range of 5,000 to 500,000 Daltons. The molecular weight of a polymer is also important from the point of view that molecular weight influences the biodegradation rate of the polymer. For a diffusional mechanism of drug release, the polymer should remain intact until all of the drug is released from the microparticles and then degrade. The drug is also released from the microparticles as the polymeric excipient bioerodes. By an appropriate selection of polymeric materials a microsphere composition is made such that the resulting microspheres exhibit both diffusional release and biodegradation release properties. This is useful in affording multiphasic release patterns.

A variety of methods are known by that compounds are encapsulated in microspheres. In these methods, the anti-apoptotic agent or pro-apoptotic agent is generally dispersed or emulsified, using stirrers, agitators, or other dynamic mixing techniques, in a solvent containing a wall-forming material. Solvent is then removed from the microspheres, and thereafter the microsphere product is obtained.

In one embodiment, controlled-release apoptosis modulating compositions are made through the incorporation of the anti-apoptotic agent or pro-apoptotic agents and/or other pharmaceutical agents into ethylene-vinyl acetate copolymer matrices. (See U.S. Pat. No. 6,083,534, incorporated herein for such disclosure). In another embodiment, anti-apoptotic agent or pro-apoptotic agents are incorporated into poly (lactic-glycolic acid) or poly-L-lactic acid microspheres. Id. In yet another embodiment, the anti-apoptotic agent or pro-apoptotic agents are encapsulated into alginate microspheres. (See U.S. Pat. No. 6,036,978, incorporated herein for such disclosure). Biocompatible methacrylate-based polymers to encapsulate the apoptosis modulating compounds or compositions are optionally used in the compositions and methods disclosed herein. A wide range of methacrylate-based polymer systems are commercially available, such as the EUDRAGIT polymers marketed by Evonik. One useful aspect of methacrylate polymers is that the properties of the composition are varied by incorporating various co-polymers. For example, poly(acrylic acid-co-methylmethacrylate) microparticles exhibit enhanced mucoadhesion properties as the carboxylic acid groups in the poly(acrylic acid) form hydrogen bonds with mucin (Park et al, Pharm. Res. (1987) 4(6):457-464). Variation of the ratio between acrylic acid and methylmethacrylate monomers serves to modulate the properties of the co-polymer. Methacrylate-based microparticles have also been used in protein therapeutic compositions (Naha et al, Journal of Microencapsulation 4 Feb. 2008 (online publication)). In one embodiment, the enhanced viscosity auris-acceptable compositions described herein comprise apoptosis modulating microspheres wherein the microspheres are formed from a methacrylate polymer or copolymer. In an additional embodiment, the enhanced viscosity composition described herein comprises apoptosis modulating microspheres wherein the microspheres are mucoadhesive. Other controlled-release systems, including incorporation or deposit of polymeric materials or matrices onto solid or hollow spheres containing anti-apoptotic agent or pro-apoptotic agents, are also explicitly contemplated within the embodiments disclosed herein. The types of controlled-release systems available without significantly losing activity of the anti-apoptotic agent or pro-apoptotic agent are determined using the teachings, examples, and principles disclosed herein

An example of a conventional microencapsulation process for pharmaceutical preparations is shown in U.S. Pat. No. 3,737,337, incorporated herein by reference for such disclosure. The apoptosis modulating substances to be encapsulated or embedded are dissolved or dispersed in the organic solution of the polymer (phase A), using conventional mixers, including (in the preparation of dispersion) vibrators and high-speed stirrers, etc. The dispersion of phase (A), containing the core material in solution or in suspension, is carried out in the aqueous phase (B), again using conventional mixers, such as high-speed mixers, vibration mixers, or even spray nozzles, in that case the particle size of the microspheres will be determined not only by the concentration of phase (A), but also by the emulsate or microsphere size. With conventional techniques for the microencapsulation of an anti-apoptotic agent or pro-apoptotic agents, the microspheres form when the solvent containing an active agent and a polymer is emulsified or dispersed in an immiscible solution by stirring, agitating, vibrating, or some other dynamic mixing technique, often for a relatively long period of time.

Methods for the construction of microspheres are also described in U.S. Pat. No. 4,389,330, and U.S. Pat. No. 4,530,840, incorporated herein by reference for such disclosure. The desired anti-apoptotic agent or pro-apoptotic agent is dissolved or dispersed in an appropriate solvent. To the agent-containing medium is added the polymeric matrix material in an amount relative to the active ingredient that gives a product of the desired loading of active agent. Optionally, all of the ingredients of the apoptosis modulating microsphere product can be blended in the solvent medium together. Suitable solvents for the agent and the polymeric matrix material include organic solvents such as acetone, halogenated hydrocarbons such as chloroform, methylene chloride and the like, aromatic hydrocarbon compounds, halogenated aromatic hydrocarbon compounds, cyclic ethers, alcohols, ethyl acetate and the like.

The mixture of ingredients in the solvent is emulsified in a continuous-phase processing medium; the continuous-phase medium being such that a dispersion of microdroplets containing the indicated ingredients is formed in the continuous-phase medium. Naturally, the continuous-phase processing medium and the organic solvent must be immiscible, and includes water although nonaqueous media such as xylene and toluene and synthetic oils and natural oils are optionally used. Optionally, a surfactant is added to the continuous-phase processing medium to prevent the microparticles from agglomerating and to control the size of the solvent microdroplets in the emulsion. A preferred surfactant-dispersing medium combination is a 1 to 10 wt. % poly (vinyl alcohol) in water mixture. The dispersion is formed by mechanical agitation of the mixed materials. An emulsion is optionally formed by adding small drops of the active agent-wall forming material solution to the continuous phase processing medium. The temperature during the formation of the emulsion is not especially critical but influences the size and quality of the microspheres and the solubility of the drug in the continuous phase. It is desirable to have as little of the agent in the continuous phase as possible. Moreover, depending on the solvent and continuous-phase processing medium employed, the temperature must not be too low or the solvent and processing medium will solidify or the processing medium will become too viscous for practical purposes, or too high that the processing medium will evaporate, or that the liquid processing medium will not be maintained. Moreover, the temperature of the medium cannot be so high that the stability of the particular agent being incorporated in the microspheres is adversely affected. Accordingly, the dispersion process is conducted at any temperature that maintains stable operating conditions, which preferred temperature being about 15° C. to 60° C., depending upon the drug and excipient selected.

The dispersion that is formed is a stable emulsion and from this dispersion the organic solvent immiscible fluid is optionally partially removed in the first step of the solvent removal process. The solvent is removed by techniques such as heating, the application of a reduced pressure or a combination of both. The temperature employed to evaporate solvent from the microdroplets is not critical, but should not be that high that it degrades the anti-apoptotic agent or pro-apoptotic agent employed in the preparation of a given microparticle, nor should it be so high as to evaporate solvent at such a rapid rate to cause defects in the wall forming material. Generally, from 5 to 75%, of the solvent is removed in the first solvent removal step.

After the first stage, the dispersed microparticles in the solvent immiscible fluid medium are isolated from the fluid medium by any convenient means of separation. Thus, for example, the fluid is decanted from the microsphere or the microsphere suspension is filtered. Still other, various combinations of separation techniques are used if desired.

Following the isolation of the microspheres from the continuous-phase processing medium, the remainder of the solvent in the microspheres is removed by extraction. In this step, the microspheres are suspended in the same continuous-phase processing medium used in step one, with or without surfactant, or in another liquid. The extraction medium removes the solvent from the microspheres and yet does not dissolve the microspheres. During the extraction, the extraction medium with dissolved solvent is optionally removed and replaced with fresh extraction medium. This is best done on a continual basis. The rate of extraction medium replenishment of a given process is a variable that is determined at the time the process is performed and, therefore, no precise limits for the rate must be predetermined. After the majority of the solvent has been removed from the microspheres, the microspheres are dried by exposure to air or by other conventional drying techniques such as vacuum drying, drying over a desiccant, or the like. This process is very efficient in encapsulating the anti-apoptotic agent or pro-apoptotic agent since core loadings of up to 80 wt. %, preferably up to 60 wt. % are obtained.

Alternatively, controlled-release microspheres containing an anti-apoptotic agent or pro-apoptotic agent is prepared through the use of static mixers. Static or motionless mixers consist of a conduit or tube in that is received a number of static mixing agents. Static mixers provide homogeneous mixing in a relatively short length of conduit, and in a relatively short period of time. With static mixers the fluid moves through the mixer rather than some part of the mixer, such as a blade, moving through the fluid.

A static mixer is optionally used to create an emulsion. When using a static mixer to form an emulsion, several factors determine emulsion particle size, including the density and viscosity of the various solutions or phases to be mixed, volume ratio of the phases, interfacial tension between the phases, static mixer parameters (conduit diameter; length of mixing element; number of mixing elements), and linear velocity through the static mixer. Temperature is a variable because it affects density, viscosity, and interfacial tension. The controlling variables are linear velocity, sheer rate, and pressure drop per unit length of static mixer.

In order to create microspheres containing an anti-apoptotic agent or pro-apoptotic agent using a static mixer process, an organic phase and an aqueous phase are combined. The organic and aqueous phases are largely or substantially immiscible, with the aqueous phase constituting the continuous phase of the emulsion. The organic phase includes an anti-apoptotic agent or pro-apoptotic agent as well as a wall-forming polymer or polymeric matrix material. The organic phase is prepared by dissolving an anti-apoptotic agent or pro-apoptotic agent in an organic or other suitable solvent, or by forming a dispersion or an emulsion containing the anti-apoptotic agent or pro-apoptotic agent. The organic phase and the aqueous phase are pumped so that the two phases flow simultaneously through a static mixer, thereby forming an emulsion that comprises microspheres containing the anti-apoptotic agent or pro-apoptotic agent encapsulated in the polymeric matrix material. The organic and aqueous phases are pumped through the static mixer into a large volume of quench liquid to extract or remove the organic solvent. Organic solvent is optionally removed from the microspheres while they are washing or being stirred in the quench liquid. After the microspheres are washed in a quench liquid, they are isolated, as through a sieve, and dried.

In one embodiment, microspheres are prepared using a static mixer. The process is not limited to the solvent extraction technique discussed above, but is used with other encapsulation techniques. For example, the process is optionally used with a phase separation encapsulation technique. To do so, an organic phase is prepared that comprises an anti-apoptotic agent or pro-apoptotic agent suspended or dispersed in a polymer solution. The non-solvent second phase is free from solvents for the polymer and active agent. A preferred non-solvent second phase is silicone oil. The organic phase and the non-solvent phase are pumped through a static mixer into a non-solvent quench liquid, such as heptane. The semi-solid particles are quenched for complete hardening and washing. The process of microencapsulation includes spray drying, solvent evaporation, a combination of evaporation and extraction, and melt extrusion.

In another embodiment, the microencapsulation process involves the use of a static mixer with a single solvent. This process is described in detail in U.S. application Ser. No. 08/338,805, herein incorporated by reference for such disclosure. An alternative process involves the use of a static mixer with co-solvents. In this process, biodegradable microspheres comprising a biodegradable polymeric binder and an anti-apoptotic agent or pro-apoptotic agent are prepared, which comprises a blend of at least two substantially non-toxic solvents, free of halogenated hydrocarbons to dissolve both the agent and the polymer. The solvent blend containing the dissolved agent and polymer is dispersed in an aqueous solution to form droplets. The resulting emulsion is then added to an aqueous extraction medium preferably containing at least one of the solvents of the blend, whereby the rate of extraction of each solvent is controlled, whereupon the biodegradable microspheres containing the pharmaceutically active agent are formed. This process has the advantage that less extraction medium is required because the solubility of one solvent in water is substantially independent of the other and solvent selection is increased, especially with solvents that are particularly difficult to extract.

Nanoparticles are also contemplated for use with the anti-apoptotic agent or pro-apoptotic agents disclosed herein. Nanoparticles are material structures of about 100 nm or less in size. One use of nanoparticles in pharmaceutical compositions is the formation of suspensions as the interaction of the particle surface with solvent is strong enough to overcome differences in density. Nanoparticle suspensions are sterilized as the nanoparticles are small enough to be subjected to sterilizing filtration (see, e.g., U.S. Pat. No. 6,139,870, herein incorporated by reference for such disclosure). Nanoparticles comprise at least one hydrophobic, water-insoluble and water-indispersible polymer or copolymer emulsified in a solution or aqueous dispersion of surfactants, phospholipids or fatty acids. The anti-apoptotic agent or pro-apoptotic agent is optionally introduced with the polymer or the copolymer into the nanoparticles.

Lipid nanocapsules as controlled-release structures, as well for penetrating the round window membrane and reaching auris interna and/or auris media targets, is also contemplated herein. Lipid nanocapsules are optionally formed by emulsifying capric and caprylic acid triglycerides (Labrafac WL 1349; avg. mw 512), soybean lecithin (LIPOID® S75-3; 69% phosphatidylcholine and other phospholipids), surfactant (for example, Solutol HS15), a mixture of polyethylene glycol 660 hydroxystearate and free polyethylene glycol 660; NaCl and water. The mixture is stirred at room temperature to obtain an oil emulsion in water. After progressive heating at a rate of 4° C./min under magnetic stirring, a short interval of transparency should occur close to 70° C., and the inverted phase (water droplets in oil) obtained at 85° C. Three cycles of cooling and heating is then applied between 85° C. and 60° C. at the rate of 4° C./min, and a fast dilution in cold water at a temperature close to 0° C. to produce a suspension of nanocapsules. To encapsulate the anti-apoptotic agent or pro-apoptotic agents, the agent is optionally added just prior to the dilution with cold water.

In some embodiments, anti-apoptotic agent or pro-apoptotic agents are inserted into the lipid nanocapsules by incubation for 90 minutes with an aqueous micellar solution of the auris active agent. The suspension is then vortexed every 15 minutes, and then quenched in an ice bath for 1 minute.

Suitable auris-acceptable surfactants are, by way of example, cholic acid or taurocholic acid salts. Taurocholic acid, the conjugate formed from cholic acid and taurine, is a fully metabolizable sulfonic acid surfactant. An analog of taurocholic acid, tauroursodeoxycholic acid (TUDCA), is a naturally occurring bile acid and is a conjugate of taurine and ursodeoxycholic acid (UDCA). Other naturally occurring anionic (e.g., galactocerebroside sulfate), neutral (e.g., lactosylceramide) or zwitterionic surfactants (e.g., sphingomyelin, phosphatidyl choline, palmitoyl carnitine) are optionally used to prepare nanoparticles.

The auris-acceptable phospholipids are chosen, by way of example, from natural, synthetic or semi-synthetic phospholipids; lecithins (phosphatidylcholine) such as, for example, purified egg or soya lecithins (lecithin E100, lecithin E80 and phospholipons, for example phospholipon 90), phosphatidylethanolamine, phosphatidylserine, phosphatidylinositol, phosphatidylglycerol, dipalmitoylphosphatidylcholine, dipalmitoylglycerophosphatidylcholine, dimyristoylphosphatidylcholine, distearoylphosphatidylcholine and phosphatidic acid or mixtures thereof are used more particularly.

Fatty acids for use with the auris-acceptable compositions are chosen from, by way of example, lauric acid, mysristic acid, palmitic acid, stearic acid, isostearic acid, arachidic acid, behenic acid, oleic acid, myristoleic acid, palmitoleic acid, linoleic acid, alpha-linoleic acid, arachidonic acid, eicosapentaenoic acid, erucic acid, docosahexaenoic acid, and the like.

Suitable auris-acceptable surfactants are selected from known organic and inorganic pharmaceutical excipients. Such excipients include various polymers, low molecular weight oligomers, natural products, and surfactants. Preferred surface modifiers include nonionic and ionic surfactants. Two or more surface modifiers are used in combination.

Representative examples of auris-acceptable surfactants include cetyl pyridinium chloride, gelatin, casein, lecithin (phosphatides), dextran, glycerol, gum acacia, cholesterol, tragacanth, stearic acid, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters; dodecyl trimethyl ammonium bromide, polyoxyethylenestearates, colloidal silicon dioxide, phosphates, sodium dodecylsulfate, carboxymethylcellulose calcium, hydroxypropyl cellulose (HPC, HPC-SL, and HPC-L), hydroxypropyl methylcellulose (HPMC), carboxymethylcellulose sodium, methylcellulose, hydroxyethylcellulose, hydroxypropylcellulose, hydroxypropylmethyl-cellulose phthalate, noncrystalline cellulose, magnesium aluminum silicate, triethanolamine, polyvinyl alcohol (PVA), polyvinylpyrrolidone (PVP), 4-(1,1,3,3-tetaamethylbutyl)-phenol polymer with ethylene oxide and formaldehyde (also known as tyloxapol, superione, and triton), poloxamers, poloxamines, a charged phospholipid such as dimyristoyl phophatidyl glycerol, dioctylsulfosuccinate (DOSS); Tetronic® 1508, dialkylesters of sodium sulfosuccinic acid, Duponol P, Tritons X-200, Crodestas F-110, p-isononylphenoxypoly-(glycidol), Crodestas SL-40 (Croda, Inc.); and SA9OHCO, which is C₁₈H₃₇CH₂ (CON(CH₃)—CH₂ (CHOH)₄ (CH₂OH)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; and the like. Most of these surfactants are known pharmaceutical excipients and are described in detail in the Handbook of Pharmaceutical Excipients, published jointly by the American Pharmaceutical Association and The Pharmaceutical Society of Great Britain (The Pharmaceutical Press, 1986), specifically incorporated by reference for such disclosure.

The hydrophobic, water-insoluble and water-indispersible polymer or copolymer may be chosen from biocompatible and biodegradable polymers, for example lactic or glycolic acid polymers and copolymers thereof, or polylactic/polyethylene (or polypropylene) oxide copolymers, preferably with molecular weights of between 1000 and 200,000, polyhydroxybutyric acid polymers, polylactones of fatty acids containing at least 12 carbon atoms, or polyanhydrides.

The nanoparticles may be obtained by coacervation, or the technique of evaporation of solvent, from an aqueous dispersion or solution of phospholipids and of an oleic acid salt into that is added an immiscible organic phase comprising the active principle and the hydrophobic, water-insoluble and water-indispersible polymer or copolymer. The mixture is pre-emulsified and then subjected to homogenization and evaporation of the organic solvent to obtain an aqueous suspension of very small-sized nanoparticles.

A variety of methods are optionally employed to fabricate the apoptosis modulating nanoparticles that are within the scope of the embodiments. These methods include vaporization methods, such as free jet expansion, laser vaporization, spark erosion, electro explosion and chemical vapor deposition; physical methods involving mechanical attrition (e.g., “pearlmilling” technology, Elan Nanosystems), super critical CO2 and interfacial deposition following solvent displacement. In one embodiment, the solvent displacement method is used. The size of nanoparticles produced by this method is sensitive to the concentration of polymer in the organic solvent; the rate of mixing; and to the surfactant employed in the process. Continuous flow mixers provide the necessary turbulence to ensure small particle size. One type of continuous flow mixing device that is optionally used to prepare nanoparticles has been described (Hansen et al J Phys Chem 92, 2189-96, 1988). In other embodiments, ultrasonic devices, flow through homogenizers or supercritical CO2 devices may be used to prepare nanoparticles.

If suitable nanoparticle homogeneity is not obtained on direct synthesis, then size-exclusion chromatography is used to produce highly uniform drug-containing particles that are freed of other components involved in their fabrication. Size-exclusion chromatography (SEC) techniques, such as gel-filtration chromatography, is used to separate particle-bound anti-apoptotic agent or pro-apoptotic agent or other pharmaceutical compound from free anti-apoptotic agent or pro-apoptotic agent or other pharmaceutical compound, or to select a suitable size range of apoptosis modulating-containing nanoparticles. Various SEC media, such as Superdex 200, Superose 6, Sephacryl 1000 are commercially available and are employed for the size-based fractionation of such mixtures. Additionally, nanoparticles are optionally purified by centrifugation, membrane filtration and by use of other molecular sieving devices, crosslinked gels/materials and membranes.

Auris-Acceptable Cyclodextrin and Other Stabilizing Compositions

In a specific embodiment, the auris-acceptable compositions alternatively comprise a cyclodextrin. Cyclodextrins are cyclic oligosaccharides containing 6, 7, or 8 glucopyranose units, referred to as α-cyclodextrin, β-cyclodextrin, or γ-cyclodextrin respectively. Cyclodextrins have a hydrophilic exterior, which enhances water-soluble, and a hydrophobic interior that forms a cavity. In an aqueous environment, hydrophobic portions of other molecules often enter the hydrophobic cavity of cyclodextrin to form inclusion compounds. Additionally, cyclodextrins are also capable of other types of nonbonding interactions with molecules that are not inside the hydrophobic cavity. Cyclodextrins have three free hydroxyl groups for each glucopyranose unit, or 18 hydroxyl groups on α-cyclodextrin, 21 hydroxyl groups on β-cyclodextrin, and 24 hydroxyl groups on γ-cyclodextrin. One or more of these hydroxyl groups can be reacted with any of a number of reagents to form a large variety of cyclodextrin derivatives, including hydroxypropyl ethers, sulfonates, and sulfoalkylethers. Shown below is the structure of β-cyclodextrin and the hydroxypropyl-β-cyclodextrin (HPβCD).

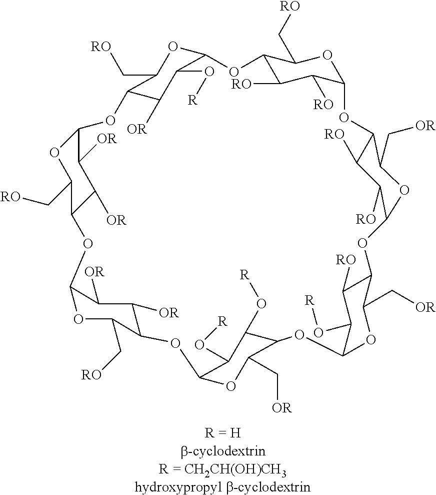

In some embodiments, the use of cyclodextrins in the pharmaceutical compositions described herein improves the solubility of the drug. Inclusion compounds are involved in many cases of enhanced solubility; however other interactions between cyclodextrins and insoluble compounds also improves solubility. Hydroxypropyl-β-cyclodextrin (HPβCD) is commercially available as a pyrogen free product. It is a nonhygroscopic white powder that readily dissolves in water. HPβCD is thermally stable and does not degrade at neutral pH. Thus, cyclodextrins improve the solubility of a therapeutic agent in a composition or composition. Accordingly, in some embodiments, cyclodextrins are included to increase the solubility of the auris-acceptable anti-apoptotic agent or pro-apoptotic agents within the compositions described herein. In other embodiments, cyclodextrins in addition serve as controlled-release excipients within the compositions described herein.

By way of example only, cyclodextrin derivatives for use include α-cyclodextrin, β-cyclodextrin, γ-cyclodextrin, hydroxyethyl β-cyclodextrin, hydroxypropyl γ-cyclodextrin, sulfated β-cyclodextrin, sulfated α-cyclodextrin, sulfobutyl ether β-cyclodextrin.

The concentration of the cyclodextrin used in the compositions and methods disclosed herein varies according to the physiochemical properties, pharmacokinetic properties, side effect or adverse events, composition considerations, or other factors associated with the therapeutically active agent, or a salt or prodrug thereof, or with the properties of other excipients in the composition. Thus, in certain circumstances, the concentration or amount of cyclodextrin used in accordance with the compositions and methods disclosed herein will vary, depending on the need. When used, the amount of cyclodextrins needed to increase solubility of the anti-apoptotic agent or pro-apoptotic agent and/or function as a controlled-release excipient in any of the compositions described herein is selected using the principles, examples, and teachings described herein.

Other stabilizers that are useful in the auris-acceptable compositions disclosed herein include, for example, fatty acids, fatty alcohols, alcohols, long chain fatty acid esters, long chain ethers, hydrophilic derivatives of fatty acids, polyvinyl pyrrolidones, polyvinyl ethers, polyvinyl alcohols, hydrocarbons, hydrophobic polymers, moisture-absorbing polymers, or combinations thereof. In some embodiments, amide analogues of stabilizers are also used. In further embodiments, the chosen stabilizer changes the hydrophobicity of the composition (e.g., oleic acid, waxes), or improves the mixing of various components in the composition (e.g., ethanol), controls the moisture level in the formula (e.g., PVP or polyvinyl pyrrolidone), controls the mobility of the phase (substances with melting points higher than room temperature such as long chain fatty acids, alcohols, esters, ethers, amides etc. or mixtures thereof; waxes), and/or improves the compatibility of the formula with encapsulating materials (e.g., oleic acid or wax). In another embodiment some of these stabilizers are used as solvents/co-solvents (e.g., ethanol). In other embodiments, stabilizers are present in sufficient amounts to inhibit the degradation of the anti-apoptotic agent or pro-apoptotic agent. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

Additional useful anti-apoptotic agent or pro-apoptotic agent auris-acceptable compositions include one or more anti-aggregation additives to enhance stability of an anti-apoptotic agent or pro-apoptotic agent compositions by reducing the rate of protein aggregation. The anti-aggregation additive selected depends upon the nature of the conditions to that the anti-apoptotic agent or pro-apoptotic agents, for example anti-apoptotic agent or pro-apoptotic agent antibodies are exposed. For example, certain compositions undergoing agitation and thermal stress require a different anti-aggregation additive than a composition undergoing lyophilization and reconstitution. Useful anti-aggregation additives include, by way of example only, urea, guanidinium chloride, simple amino acids such as glycine or arginine, sugars, polyalcohols, polysorbates, polymers such as polyethylene glycol and dextrans, alkyl saccharides, such as alkyl glycoside, and surfactants.

Other useful compositions optionally include one or more auris-acceptable antioxidants to enhance chemical stability where required. Suitable antioxidants include, by way of example only, ascorbic acid, methionine, sodium thiosulfate and sodium metabisulfite. In one embodiment, antioxidants are selected from metal chelating agents, thiol containing compounds and other general stabilizing agents.

Still other useful compositions include one or more auris-acceptable surfactants to enhance physical stability or for other purposes. Suitable nonionic surfactants include, but are not limited to, polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

In some embodiments, the auris-acceptable pharmaceutical compositions described herein are stable with respect to compound degradation over a period of any of at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 3 months, at least about 4 months, at least about 5 months, or at least about 6 months. In other embodiments, the compositions described herein are stable with respect to compound degradation over a period of at least about 1 week. Also described herein are compositions that are stable with respect to compound degradation over a period of at least about 1 month.

In other embodiments, an additional surfactant (co-surfactant) and/or buffering agent is combined with one or more of the pharmaceutically acceptable vehicles previously described herein so that the surfactant and/or buffering agent maintains the product at an optimal pH for stability. Suitable co-surfactants include, but are not limited to: a) natural and synthetic lipophilic agents, e.g., phospholipids, cholesterol, and cholesterol fatty acid esters and derivatives thereof; b) nonionic surfactants, which include for example, polyoxyethylene fatty alcohol esters, sorbitan fatty acid esters (Spans), polyoxyethylene sorbitan fatty acid esters (e.g., polyoxyethylene (20) sorbitan monooleate (Tween 80), polyoxyethylene (20) sorbitan monostearate (Tween 60), polyoxyethylene (20) sorbitan monolaurate (Tween 20) and other Tweens, sorbitan esters, glycerol esters, e.g., Myrj and glycerol triacetate (triacetin), polyethylene glycols, cetyl alcohol, cetostearyl alcohol, stearyl alcohol, polysorbate 80, poloxamers, poloxamines, polyoxyethylene castor oil derivatives (e.g., Cremophor® RH40, Cremphor A25, Cremphor A20, Cremophor® EL) and other Cremophors, sulfosuccinates, alkyl sulphates (SLS); PEG glyceryl fatty acid esters such as PEG-8 glyceryl caprylate/caprate (Labrasol), PEG-4 glyceryl caprylate/caprate (Labrafac Hydro WL 1219), PEG-32 glyceryl laurate (Gelucire 444/14), PEG-6 glyceryl mono oleate (Labrafil M 1944 CS), PEG-6 glyceryl linoleate (Labrafil M 2125 CS); propylene glycol mono- and di-fatty acid esters, such as propylene glycol laurate, propylene glycol caprylate/caprate; Brij® 700, ascorbyl-6-palmitate, stearylamine, sodium lauryl sulfate, polyoxethyleneglycerol triiricinoleate, and any combinations or mixtures thereof; c) anionic surfactants include, but are not limited to, calcium carboxymethylcellulose, sodium carboxymethylcellulose, sodium sulfosuccinate, dioctyl, sodium alginate, alkyl polyoxyethylene sulfates, sodium lauryl sulfate, triethanolamine stearate, potassium laurate, bile salts, and any combinations or mixtures thereof; and d) cationic surfactants such as cetyltrimethylammonium bromide, and lauryldimethylbenzyl-ammonium chloride.

In a further embodiment, when one or more co-surfactants are utilized in the auris-acceptable compositions of the present disclosure, they are combined, e.g., with a pharmaceutically acceptable vehicle and is present in the final composition, e.g., in an amount ranging from about 0.1% to about 20%, from about 0.5% to about 10%.

In one embodiment, the surfactant has an HLB value of 0 to 20. In additional embodiments, the surfactant has an HLB value of 0 to 3, of 4 to 6, of 7 to 9, of 8 to 18, of 13 to 15, of 10 to 18.

In one embodiment, diluents are also used to stabilize the anti-apoptotic agent or pro-apoptotic agent or other pharmaceutical compounds because they provide a more stable environment. Salts dissolved in buffered solutions (that also can provide pH control or maintenance) are utilized as diluents, including, but not limited to a phosphate buffered saline solution. In other embodiments, the gel composition is isotonic with the endolymph or the perilymph: depending on the portion of the cochlea that the anti-apoptotic agent or pro-apoptotic agent composition is targeted. Isotonic compositions are provided by the addition of a tonicity agent. Suitable tonicity agents include, but are not limited to any pharmaceutically acceptable sugar, salt or any combinations or mixtures thereof, such as, but not limited to dextrose and sodium chloride. In further embodiments, the tonicity agents are present in an amount from about 100 mOsm/kg to about 500 mOsm/kg. In some embodiments, the tonicity agent is present in an amount from about 200 mOsm/kg to about 400 mOsm/kg, from about 280 mOsm/kg to about 320 mOsm/kg. The amount of tonicity agents will depend on the target structure of the pharmaceutical composition, as described herein.

Useful tonicity compositions also include one or more salts in an amount required to bring osmolality of the composition into an acceptable range for the perilymph or the endolymph. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some embodiments, the auris-acceptable gel compositions disclosed herein alternatively or additionally contain preservatives to prevent microbial growth. Suitable auris-acceptable preservatives for use in the enhanced viscosity compositions described herein include, but are not limited to benzoic acid, boric acid, p-hydroxybenzoates, alcohols, quarternary compounds, stabilized chlorine dioxide, mercurials, such as merfen and thiomersal, mixtures of the foregoing and the like.

In a further embodiment, the preservative is, by way of example only, an antimicrobial agent, within the auris-acceptable compositions presented herein. In one embodiment, the composition includes a preservative such as by way of example only, methyl paraben, sodium bisulfite, sodium thiosulfate, ascorbate, chorobutanol, thimerosal, parabens, benzyl alcohol, phenylethanol and others. In another embodiment, the methyl paraben is at a concentration of about 0.05% to about 1.0%, about 0.1% to about 0.2%. In a further embodiment, the gel is prepared by mixing water, methylparaben, hydroxyethylcellulose and sodium citrate. In a further embodiment, the gel is prepared by mixing water, methylparaben, hydroxyethylcellulose and sodium acetate. In a further embodiment, the mixture is sterilized by autoclaving at 120° C. for about 20 minutes, and tested for pH, methylparaben concentration and viscosity before mixing with the appropriate amount of the anti-apoptotic agent or pro-apoptotic agent disclosed herein.

Suitable auris-acceptable water soluble preservatives that are employed in the drug delivery vehicle include sodium bisulfite, sodium thiosulfate, ascorbate, chorobutanol, thimerosal, parabens, benzyl alcohol, Butylated hydroxytoluene (BHT), phenylethanol and others. These agents are present, generally, in amounts of about 0.001% to about 5% by weight and, preferably, in the amount of about 0.01 to about 2% by weight. In some embodiments, auris-compatible compositions described herein are free of preservatives.

Round Window Membrane Penetration Enhancers

In another embodiment, the composition further comprises one or more round window membrane penetration enhancers. Penetration across the round window membrane is enhanced by the presence of round window membrane penetration enhancers. Round window membrane penetration enhancers are chemical entities that facilitate transport of coadministered substances across the round window membrane. Round window membrane penetration enhancers are grouped according to chemical structure. Surfactants, both ionic and non-ionic, such as sodium lauryl sulfate, sodium laurate, polyoxyethylene-20-cetyl ether, laureth-9, sodium dodecylsulfate, dioctyl sodium sulfosuccinate, polyoxyethylene-9-lauryl ether (PLE), Tween® 80, nonylphenoxypolyethylene NP—POE), polysorbates and the like, function as round window membrane penetration enhancers. Bile salts (such as sodium glycocholate, sodium deoxycholate, sodium taurocholate, sodium taurodihydrofusidate, sodium glycodihydrofusidate and the like), fatty acids and derivatives (such as oleic acid, caprylic acid, mono- and di-glycerides, lauric acids, acylcholines, caprylic acids, acylcarnitines, sodium caprates and the like), chelating agents (such as EDTA, citric acid, salicylates and the like), sulfoxides (such as dimethyl sulfoxide (DMSO), decylmethyl sulfoxide and the like), and alcohols (such as ethanol, isopropanol, glycerol, propanediol and the like) also function as round window membrane penetration enhancers.

In some embodiments, the auris acceptable penetration enhancer is a surfactant comprising an alkyl-glycoside wherein the alkyl glycoside is tetradecyl-β-D-maltoside. In some embodiments, the auris acceptable penetration enhancer is a surfactant comprising an alkyl-glycoside wherein the alkyl glycoside is dodecyl-maltoside. In certain instances, the penetration enhancing agent is a hyaluronidase. In certain instances, a hyaluronidase is a human or bovine hyaluronidase. In some instances, a hyaluronidase is a human hyaluronidase (e.g., hyaluronidase found in human sperm, PH20 (Halozyme), Hyelenex® (Baxter International, Inc.)). In some instances, a hyaluronidase is a bovine hyaluronidase (e.g., bovine testicular hyaluronidase, Amphadase® (Amphastar Pharmaceuticals), Hydase® (PrimaPharm, Inc). In some instances, a hyaluronidase is an ovine hyaluronidase, Vitrase® (ISTA Pharmaceuticals). In certain instances, a hyaluronidase described herein is a recombinant hyaluronidase. In some instances, a hyaluronidase described herein is a humanized recombinant hyaluronidase. In some instances, a hyaluronidase described herein is a pegylated hyaluronidase (e.g., PEGPH20 (Halozyme)). In addition, the peptide-like penetration enhancers described in U.S. Pat. Nos. 7,151,191, 6,221,367 and 5,714,167, herein incorporated by references for such disclosure, are contemplated as an additional embodiment. These penetration enhancers are amino-acid and peptide derivatives and enable drug absorption by passive transcellular diffusion without affecting the integrity of membranes or intercellular tight junctions.

Round Window Membrane Permeable Liposomes

Liposomes or lipid particles may also be employed to encapsulate the anti-apoptotic agent or pro-apoptotic agent compositions or compositions. Phospholipids that are gently dispersed in an aqueous medium form multilayer vesicles with areas of entrapped aqueous media separating the lipid layers. Sonication, or turbulent agitation, of these multilayer vesicles results in the formation of single layer vesicles, commonly referred to as liposomes, with sizes of about 10-1000 nm. These liposomes have many advantages as anti-apoptotic agent or pro-apoptotic agents or other pharmaceutical agent carriers. They are biologically inert, biodegradable, non-toxic and non-antigenic. Liposomes are formed in various sizes and with varying compositions and surface properties. Additionally, they are able to entrap a wide variety of agents and release the agent at the site of liposome collapse.

Suitable phospholipids for use in auris-acceptable liposomes here are, for example, phosphatidyl cholines, ethanolamines and serines, sphingomyelins, cardiolipins, plasmalogens, phosphatidic acids and cerebrosides, in particular those that are soluble together with the anti-apoptotic agent or pro-apoptotic agents herein in non-toxic, pharmaceutically acceptable organic solvents. Preferred phospholipids are, for example, phosphatidyl choline, phosphatidyl ethanolmine, phosphatidyl serine, phosphatidyl inositol, lysophosphatidyl choline, phosphatidyl glycerol and the like, and mixtures thereof especially lecithin, e.g. soya lecithin. The amount of phospholipid used in the present composition range from about 10 to about 30%, preferably from about 15 to about 25% and in particular is about 20%.

Lipophilic additives may be employed advantageously to modify selectively the characteristics of the liposomes. Examples of such additives include by way of example only, stearylamine, phosphatidic acid, tocopherol, cholesterol, cholesterol hemisuccinate and lanolin extracts. The amount of lipophilic additive used range from 0.5 to 8%, preferably from 1.5 to 4% and in particular is about 2%. Generally, the ratio of the amount of lipophilic additive to the amount of phospholipid ranges from about 1:8 to about 1:12 and in particular is about 1:10. Said phospholipid, lipophilic additive and the anti-apoptotic agent or pro-apoptotic agent and other pharmaceutical compounds are employed in conjunction with a non-toxic, pharmaceutically acceptable organic solvent system that dissolve said ingredients. Said solvent system not only must dissolve the anti-apoptotic agent or pro-apoptotic agent completely, but it also has to allow the composition of stable single bilayered liposomes. The solvent system comprises dimethylisosorbide and tetraglycol (glycofurol, tetrahydrofurfuryl alcohol polyethylene glycol ether) in an amount of about 8 to about 30%. In said solvent system, the ratio of the amount of dimethylisosorbide to the amount of tetraglycol range from about 2:1 to about 1:3, in particular from about 1:1 to about 1:2.5 and preferably is about 1:2. The amount of tetraglycol in the final composition thus varies from 5 to 20%, in particular from 5 to 15% and preferably is approximately 10%. The amount of dimethylisosorbide in the final composition thus ranges from 3 to 10%, in particular from 3 to 7% and preferably is approximately 5%.

The term “organic component” as used hereinafter refers to mixtures comprising said phospholipid, lipophilic additives and organic solvents. The anti-apoptotic agent or pro-apoptotic agent may be dissolved in the organic component, or other means to maintain full activity of the agent. The amount of an anti-apoptotic agent or pro-apoptotic agent in the final composition may range from 0.1 to 5.0%. In addition, other ingredients such as anti-oxidants may be added to the organic component. Examples include tocopherol, butylated hydroxyanisole, butylated hydroxytoluene, ascorbyl palmitate, ascorbyl oleate and the like.

Liposomal compositions are alternatively prepared, for anti-apoptotic agent or pro-apoptotic agents or other pharmaceutical agents that are moderately heat-resistant, by (a) heating the phospholipid and the organic solvent system to about 60-80° C. in a vessel, dissolving the active ingredient, then adding any additional formulating agents, and stirring the mixture until complete dissolution is obtained; (b) heating the aqueous solution to 90-95° C. in a second vessel and dissolving the preservatives therein, allowing the mixture to cool and then adding the remainder of the auxiliary formulating agents and the remainder of the water, and stirring the mixture until complete dissolution is obtained; thus preparing the aqueous component; (c) transferring the organic phase directly into the aqueous component, while homogenizing the combination with a high performance mixing apparatus, for example, a high-shear mixer; and (d) adding a viscosity enhancing agent to the resulting mixture while further homogenizing. The aqueous component is optionally placed in a suitable vessel that is equipped with a homogenizer and homogenization is effected by creating turbulence during the injection of the organic component. Any mixing means or homogenizer that exerts high shear forces on the mixture may be employed. Generally, a mixer capable of speeds from about 1,500 to 20,000 rpm, in particular from about 3,000 to about 6,000 rpm may be employed. Suitable viscosity enhancing agents for use in process step (d) are for example, xanthan gum, hydroxypropyl cellulose, hydroxypropyl methylcellulose or mixtures thereof. The amount of viscosity enhancing agent depends on the nature and the concentration of the other ingredients and in general ranges from about 0.5 to 2.0%, or approximately 1.5%. In order to prevent degradation of the materials used during the preparation of the liposomal composition, it is advantageous to purge all solutions with an inert gas such as nitrogen or argon, and to conduct all steps under an inert atmosphere. Liposomes prepared by the above described method usually contain most of the active ingredient bound in the lipid bilayer and separation of the liposomes from unencapsulated material is not required.

In other embodiments, the auris-acceptable compositions, including gel compositions and viscosity-enhanced compositions, further include excipients, other medicinal or pharmaceutical agents, carriers, adjuvants, such as preserving, stabilizing, wetting or emulsifying agents, solution promoters, salts, solubilizers, an antifoaming agent, an antioxidant, a dispersing agent, a wetting agent, a surfactant, or combinations thereof.

Suitable carriers for use in an auris-acceptable composition described herein include, but are not limited to, any pharmaceutically acceptable solvent compatible with the targeted auris structure's physiological environment. In other embodiments, the base is a combination of a pharmaceutically acceptable surfactant and solvent.

In some embodiments, other excipients include, sodium stearyl fumarate, diethanolamine cetyl sulfate, isostearate, polyethoxylated castor oil, nonoxyl 10, octoxynol 9, sodium lauryl sulfate, sorbitan esters (sorbitan monolaurate, sorbitan monooleate, sorbitan monopalmitate, sorbitan monostearate, sorbitan sesquioleate, sorbitan trioleate, sorbitan tristearate, sorbitan laurate, sorbitan oleate, sorbitan palmitate, sorbitan stearate, sorbitan dioleate, sorbitan sesqui-isostearate, sorbitan sesquistearate, sorbitan tri-isostearate), lecithin pharmaceutical acceptable salts thereof or combinations or mixtures thereof.

In other embodiments, the carrier is a polysorbate. Polysorbates are nonionic surfactants of sorbitan esters. Polysorbates useful in the present disclosure include, but are not limited to polysorbate 20, polysorbate 40, polysorbate 60, polysorbate 80 (Tween 80) and any combinations or mixtures thereof. In further embodiments, polysorbate 80 is utilized as the pharmaceutically acceptable carrier.

In one embodiment, water-soluble glycerin-based auris-acceptable enhanced viscosity compositions utilized in the preparation of pharmaceutical delivery vehicles comprise at least one anti-apoptotic agent or pro-apoptotic agent containing at least about 0.1% of the water-soluble glycerin compound or more. In some embodiments, the percentage of an anti-apoptotic agent or pro-apoptotic agent is varied between about 1% and about 95%, between about 5% and about 80%, between about 10% and about 60% or more of the weight or volume of the total pharmaceutical composition. In some embodiments, the amount of the compound(s) in each therapeutically useful anti-apoptotic agent or pro-apoptotic agent composition is prepared in such a way that a suitable dosage will be obtained in any given unit dose of the compound. Factors such as solubility, bioavailability, biological half-life, route of administration, product shelf life, as well as other pharmacological considerations are contemplated herein.

If desired, the auris-acceptable pharmaceutical gels also contain co-solvents, preservatives, cosolvents, ionic strength and osmolality adjustors and other excipients in addition to buffering agents. Suitable auris-acceptable water soluble buffering agents are alkali or alkaline earth metal carbonates, phosphates, bicarbonates, citrates, borates, acetates, succinates and the like, such as sodium phosphate, citrate, borate, acetate, bicarbonate, carbonate and tromethamine (TRIS). These agents are present in amounts sufficient to maintain the pH of the system at 7.4±0.2 and preferably, 7.4. As such, the buffering agent is as much as 5% on a weight basis of the total composition.

Cosolvents are used to enhance anti-apoptotic agent or pro-apoptotic agent solubility, however, some anti-apoptotic agent or pro-apoptotic agents or other pharmaceutical compounds are insoluble. These are often suspended in the polymer vehicle with the aid of suitable suspending or viscosity enhancing agents.

Moreover, some pharmaceutical excipients, diluents or carriers are potentially ototoxic. For example, benzalkonium chloride, a common preservative, is ototoxic and therefore potentially harmful if introduced into the vestibular or cochlear structures. In formulating a controlled-release anti-apoptotic agent or pro-apoptotic agent composition, it is advised to avoid or combine the appropriate excipients, diluents or carriers to lessen or eliminate potential ototoxic components from the composition, or to decrease the amount of such excipients, diluents or carriers. Optionally, a controlled-release anti-apoptotic agent or pro-apoptotic agent composition includes otoprotective agents, such as antioxidants, alpha lipoic acid, calcium, fosfomycin or iron chelators, to counteract potential ototoxic effects that may arise from the use of specific therapeutic agents or excipients, diluents or carriers.

The following are examples of therapeutically acceptable otic compositions:

| Example Composition | Example Characteristics |
|---|---|
| Chitosan glycerophosphate | tunable degradation of matrix in vitro |
| (CGP) | tunable TACE inhibitor release in vitro: e.g., ~50% of drug |
| | released after 24 hrs |
| | biodegradable |
| | compatible with drug delivery to the inner ear |
| | suitable for macromolecules and hydrophobic drugs |
| PEG-PLGA-PEG triblock | tunable high stability: e.g., maintains mechanical integrity >1 |
| polymers | month in vitro |
| | tunable fast release of hydrophilic drugs: e.g., ~50% of drug |
| | released after 24 hrs, and remainder released over ~5 days |
| | tunable slow release of hydrophobic drugs: e.g., ~80% |
| | released after 8 weeks |
| | biodegradable |
| | subcutaneous injection of solution: e.g., gel forms within |
| | seconds and is intact after 1 month |
| PEO-PPO-PEO triblock | Tunable sol-gel transition temperature: e.g., decreases with |
| copolymers (e.g., Pluronic | increasing F127 concentration |
| or Poloxameres) (e.g., | |
| F127) | |
| Chitosan glycerophosphate | CGP composition tolerates liposomes: e.g., up to 15 uM/ml |
| with drug-loaded liposomes | liposomes. |
| | liposomes tunably reduce drug release time (e.g., up to 2 |
| | weeks in vitro). |
| | increase in liposome diameter optionally reduces drug release |
| | kinetics (e.g., liposome size between 100 and 300 nm) |
| | release parameters are controlled by changing composition of |
| | liposomes |

The compositions disclosed herein alternatively encompass an otoprotectant agent in addition to the at least one active agent and/or excipients, including but not limited to such agents as antioxidants, alpha lipoic acid, calcium, fosfomycin or iron chelators, to counteract potential ototoxic effects that may arise from the use of specific therapeutic agents or excipients, diluents or carriers.

### Modes of Treatment

Dosing Methods and Schedules

Drugs delivered to the inner ear have been administered systemically via oral, intravenous or intramuscular routes. However, systemic administration for pathologies local to the inner ear increases the likelihood of systemic toxicities and adverse side effects and creates a non-productive distribution of drug in that high levels of drug are found in the serum and correspondingly lower levels are found at the inner ear.

Intratympanic injection of therapeutic agents is the technique of injecting a therapeutic agent behind the tympanic membrane into the middle and/or inner ear. In one embodiment, the compositions described herein are administered directly onto the round window membrane via transtympanic injection. In another embodiment, the anti-apoptotic agent or pro-apoptotic agent auris-acceptable compositions described herein are administered onto the round window membrane via a non-transtympanic approach to the inner ear. In additional embodiments, the composition described herein is administered onto the round window membrane via a surgical approach to the round window membrane comprising modification of the crista fenestrae cochleae.

In one embodiment the delivery system is a syringe and needle apparatus that is capable of piercing the tympanic membrane and directly accessing the round window membrane or crista fenestrae cochleae of the auris interna. In some embodiments, the needle on the syringe is wider than a 18 gauge needle. In another embodiment, the needle gauge is from 18 gauge to 31 gauge. In a further embodiment, the needle gauge is from 25 gauge to 30 gauge. Depending upon the thickness or viscosity of the anti-apoptotic agent or pro-apoptotic agent compositions or compositions, the gauge level of the syringe or hypodermic needle may be varied accordingly. In another embodiment, the internal diameter of the needle can be increased by reducing the wall thickness of the needle (commonly referred as thin wall or extra thin wall needles) to reduce the possibility of needle clogging while maintaining an adequate needle gauge.

In another embodiment, the needle is a hypodermic needle used for instant delivery of the gel composition. The hypodermic needle may be a single use needle or a disposable needle. In some embodiments, a syringe may be used for delivery of the pharmaceutically acceptable gel-based anti-apoptotic agent or pro-apoptotic agent-containing compositions as disclosed herein wherein the syringe has a press-fit (Luer) or twist-on (Luer-lock) fitting. In one embodiment, the syringe is a hypodermic syringe. In another embodiment, the syringe is made of plastic or glass. In yet another embodiment, the hypodermic syringe is a single use syringe. In a further embodiment, the glass syringe is capable of being sterilized. In yet a further embodiment, the sterilization occurs through an autoclave. In another embodiment, the syringe comprises a cylindrical syringe body wherein the gel composition is stored before use. In other embodiments, the syringe comprises a cylindrical syringe body wherein the anti-apoptotic agent or pro-apoptotic agent pharmaceutically acceptable gel-based compositions as disclosed herein is stored before use that conveniently allows for mixing with a suitable pharmaceutically acceptable buffer. In other embodiments, the syringe may contain other excipients, stabilizers, suspending agents, diluents or a combination thereof to stabilize or otherwise stably store the anti-apoptotic agent or pro-apoptotic agent or other pharmaceutical compounds contained therein.

In some embodiments, the syringe comprises a cylindrical syringe body wherein the body is compartmentalized in that each compartment is able to store at least one component of the auris-acceptable anti-apoptotic agent or pro-apoptotic agent gel composition. In a further embodiment, the syringe having a compartmentalized body allows for mixing of the components prior to injection into the auris media or auris interna. In other embodiments, the delivery system comprises multiple syringes, each syringe of the multiple syringes contains at least one component of the gel composition such that each component is pre-mixed prior to injection or is mixed subsequent to injection. In a further embodiment, the syringes disclosed herein comprise at least one reservoir wherein the at least one reservoir comprises an anti-apoptotic agent or pro-apoptotic agent, or a pharmaceutically acceptable buffer, or a viscosity enhancing agent, such as a gelling agent or a combination thereof. Commercially available injection devices are optionally employed in their simplest form as ready-to-use plastic syringes with a syringe barrel, needle assembly with a needle, plunger with a plunger rod, and holding flange, to perform an intratympanic injection.

In some embodiments, the delivery device is an apparatus designed for administration of therapeutic agents to the middle and/or inner ear. By way of example only: GYRUS Medical Gmbh offers micro-otoscopes for visualization of and drug delivery to the round window niche; Arenberg has described a medical treatment device to deliver fluids to inner ear structures in U.S. Pat. Nos. 5,421,818; 5,474,529; and 5,476,446, each of that is incorporated by reference herein for such disclosure. U.S. patent application Ser. No. 08/874,208, which is incorporated herein by reference for such disclosure, describes a surgical method for implanting a fluid transfer conduit to deliver therapeutic agents to the inner ear. U.S. Patent Application Publication 2007/0167918, which is incorporated herein by reference for such disclosure, further describes a combined otic aspirator and medication dispenser for intratympanic fluid sampling and medicament application.

The compositions described herein, and modes of administration thereof, are also applicable to methods of direct instillation or perfusion of the inner ear compartments. Thus, the compositions described herein are useful in surgical procedures including, by way of non-limiting examples, cochleostomy, labyrinthotomy, mastoidectomy, stapedectomy, endolymphatic sacculotomy or the like.

The auris-acceptable compositions or compositions containing the anti-apoptotic agent or pro-apoptotic agent compound(s) described herein are administered for prophylactic and/or therapeutic treatments. In therapeutic applications, the anti-apoptotic agent or pro-apoptotic agent compositions are administered to a patient already suffering from a disorder disclosed herein, in an amount sufficient to cure or at least partially arrest the symptoms of the disease, disorder or condition. Amounts effective for this use will depend on the severity and course of the disease, disorder or condition, previous therapy, the patient's health status and response to the drugs, and the judgment of the treating physician.

In the case wherein the patient's condition does not improve, upon the doctor's discretion the administration of the anti-apoptotic agent or pro-apoptotic agent compounds may be administered chronically, which is, for an extended period of time, including throughout the duration of the patient's life in order to ameliorate or otherwise control or limit the symptoms of the patient's disease or condition.

In the case wherein the patient's status does improve, upon the doctor's discretion the administration of the anti-apoptotic agent or pro-apoptotic agent compounds may be given continuously; alternatively, the dose of drug being administered may be temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday may be from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's otic conditions has occurred, a maintenance anti-apoptotic agent or pro-apoptotic agent dose is administered if necessary. Subsequently, the dosage or the frequency of administration, or both, is optionally reduced, as a function of the symptoms, to a level at that the improved disease, disorder or condition is retained. In certain embodiments, patients require intermittent treatment on a long-term basis upon any recurrence of symptoms.

The amount of an anti-apoptotic agent or pro-apoptotic agent that will correspond to such an amount will vary depending upon factors such as the particular compound, disease condition and its severity, according to the particular circumstances surrounding the case, including, e.g., the specific anti-apoptotic agent or pro-apoptotic agent being administered, the route of administration, the condition being treated, the target area being treated, and the subject or host being treated. In general, however, doses employed for adult human treatment will typically be in the range of 0.02-50 mg per administration, preferably 1-15 mg per administration. The desired dose is presented in a single dose or as divided doses administered simultaneously (or over a short period of time) or at appropriate intervals.

In some embodiments, the initial administration is a particular anti-apoptotic agent or pro-apoptotic agent and the subsequent administration a different composition or anti-apoptotic agent or pro-apoptotic agent.

Implants of Exogenous Materials

In some embodiments, the pharmaceutical formulations, compositions and devices described herein are used in combination with (e.g., implantation, short-term use, long-term use, or removal of) the implantation of an exogenous material (e.g., a medical device or a plurality of cells (e.g., stem cells)). As used herein, the term “exogenous material” includes auris-interna or auris-media medical devices (e.g., hearing sparing devices, hearing improving devices, short electrodes, micro-prostheses or piston-like prostheses); needles; drug delivery devices, and cells (e.g., stem cells). In some instances, the implants of exogenous materials are used in conjunction with a patient experiencing hearing loss. In some instances, the hearing loss is present at birth. In some instances, the hearing loss is associated with conditions that develop or progress after birth (e.g., Meniere's disease) resulting in osteoneogenesis, nerve damage, obliteration of cochlear structures, or combinations thereof.

In some instances, the exogenous material is a plurality of cells. In some instances, the exogenous material is a plurality of stem cells.

In some instances, the exogenous material is an electronic device. In some embodiments, the electronic device has an external portion placed behind the ear, and a second portion that is surgically placed under the skin that helps provide a sense of sound to a person who is profoundly deaf or severely hard-of-hearing. By way of example only, such medical device implants bypass damaged portions of the ear and directly stimulate the auditory nerve. In some instances cochlear implants are used in single sided deafness. In some instances cochlear implants are used for deafness in both ears.

In some embodiments, administration of an active agent described herein in combination with the implantation of an exogenous material (e.g., a medical device implant or a stem cell transplant) delays or prevents damage to auris structures, e.g., irritation, cell death osteoneogeneis and/or further neuronal degeneration, caused by installation of an external device and/or a plurality cells (e.g., stem cells) in the ear. In some embodiments, administration of a composition or device described herein in combination with an implant allows for a more effective restoration of hearing loss compared to an implant alone.

In some embodiments, administration of an active agent described herein reduces damage to auris structures caused by underlying conditions allowing for successful implantation. In some embodiments, administration of an active agent described herein, in conjunction surgery and/or with the implantation of an exogenous material reduces or prevents negative side-effects (e.g., cell death).

In some embodiments, administration of an active agent described herein in conjunction with the implantation of an exogenous material has a trophic effect (i.e., promotes healthy growth of cells and healing of tissue in the area of an implant or transplant). In some embodiments, a trophic effect is desirable during otic surgery or during intratympanic injection procedures. In some embodiments, a trophic effect is desirable after installation of a medical device or after a cell (e.g., stem cell) transplant. In some of such embodiments, the compositions or devices described herein are administered via direct cochlear injection, through a chochleostomy or via deposition on the round window

In some embodiments, administration of an active agent described herein reduces inflammation and/or infections associated with otic surgery, or implantation of an exogenous material (e.g., a medical device or a plurality of cells (e.g., stem cells)). In some instances, perfusion of a surgical area with a formulation described herein reduces or eliminates post-surgical and/or post-implantation complications (e.g., inflammation, hair cell damage, neuronal degeneration, osteoneogenesis or the like). In some instances, perfusion of a surgical area with a formulation described herein reduces post-surgery or post-implantation recuperation time.

In one aspect, the formulations described herein, and modes of administration thereof, are applicable to methods of direct perfusion of the inner ear compartments. Thus, the formulations described herein are useful in combination with surgical procedures including, by way of non-limiting examples, cochleostomy, labyrinthotomy, mastoidectomy, stapedectomy, stapedotomy, endolymphatic sacculotomy or the like. In some embodiments, the inner ear compartments are perfused with a formulation described herein prior to otic surgery, during otic surgery, after otic surgery, or a combination thereof. In some of such embodiments, the formulations described herein are substantially free of extended release components (e.g., gelling components such as polyoxyethylene-polyoxypropylene copolymers). In some of such embodiments, the formulations described herein contain less than 5% of the extended release components (e.g., gelling components such as polyoxyethylene-polyoxypropylene triblock copolymers) by weight of the formulation. In some of such embodiments, the formulations described herein contain less than 2% of the extended release components (e.g., gelling components such as polyoxyethylene-polyoxypropylene triblock copolymers) by weight of the formulation. In some of such embodiments, the formulations described herein contain less than 1% of the extended release components (e.g., gelling components such as polyoxyethylene-polyoxypropylene triblock copolymers) by weight of the formulation. In some of such embodiments, a composition described herein that is used for perfusion of a surgical area contains substantially no gelling component.

### Pharmacokinetics of Controlled-Release Composition or Devices

In one embodiment, a composition or device disclosed herein additionally provides an immediate release of an active agent from the composition or device, or within 1 minute, or within 5 minutes, or within 10 minutes, or within 15 minutes, or within 30 minutes, or within 60 minutes or within 90 minutes. In other embodiments, a therapeutically effective amount of an active agent is released from the composition or device immediately, or within 1 minute, or within 5 minutes, or within 10 minutes, or within 15 minutes, or within 30 minutes, or within 60 minutes or within 90 minutes. In certain embodiments the composition or device comprises an auris-pharmaceutically acceptable gel composition or device providing immediate release of active agent. Additional embodiments of the composition or device may also include an agent that enhances the viscosity of the composition or device.

In other or further embodiments, the composition or device provides an extended release composition or device of active agent. In certain embodiments, diffusion of an active agent from the composition or device occurs for a time period exceeding 5 minutes, or 15 minutes, or 30 minutes, or 1 hour, or 4 hours, or 6 hours, or 12 hours, or 18 hours, or 1 day, or 2 days, or 3 days, or 4 days, or 5 days, or 6 days, or 7 days, or 10 days, or 12 days, or 14 days, or 18 days, or 21 days, or 25 days, or 30 days, or 45 days, or 2 months or 3 months or 4 months or 5 months or 6 months or 9 months or 1 year. In other embodiments, a therapeutically effective amount of an active agent is released from the composition or device for a time period exceeding 5 minutes, or 15 minutes, or 30 minutes, or 1 hour, or 4 hours, or 6 hours, or 12 hours, or 18 hours, or 1 day, or 2 days, or 3 days, or 4 days, or 5 days, or 6 days, or 7 days, or 10 days, or 12 days, or 14 days, or 18 days, or 21 days, or 25 days, or 30 days, or 45 days, or 2 months or 3 months or 4 months or 5 months or 6 months or 9 months or 1 year.

In other embodiments, the composition or device provides both an immediate release and an extended release composition or device of active agent. In yet other embodiments, the composition or device contains a 0.25:1 ratio, or a 0.5:1 ratio, or a 1:1 ratio, or a 1:2 ratio, or a 1:3, or a 1:4 ratio, or a 1:5 ratio, or a 1:7 ratio, or a 1:10 ratio, or a 1:15 ratio, or a 1:20 ratio of immediate release and extended release composition or devices. In some embodiments, the composition or device provides an immediate release of a first active agent and an extended release of a second active agent or other active agent. In yet other embodiments, the composition or device provides an immediate release and extended release composition or device of active agent, and at least one active agent. In some embodiments, the composition or device provides a 0.25:1 ratio, or a 0.5:1 ratio, or a 1:1 ratio, or a 1:2 ratio, or a 1:3, or a 1:4 ratio, or a 1:5 ratio, or a 1:7 ratio, or a 1:10 ratio, or a 1:15 ratio, or a 1:20 ratio of immediate release and extended release composition or devices of a first active agent and second active agent, respectively.

In a specific embodiment the composition or device provides a therapeutically effective amount of an active agent at the site of disease with essentially no systemic exposure. In an additional embodiment the composition or device provides a therapeutically effective amount of an active agent at the site of disease with essentially no detectable systemic exposure. In other embodiments, the composition or device provides a therapeutically effective amount of an active agent at the site of disease with little or no detectable systemic exposure.

The combination of immediate release, delayed release and/or extended release auris compatible composition or devices may be combined with other pharmaceutical agents, as well as the excipients, diluents, stabilizers, tonicity agents and other components disclosed herein. As such, depending upon the active agent used, the thickness or viscosity desired, or the mode of delivery chosen, alternative aspects of the embodiments disclosed herein are combined with the immediate release, delayed release and/or extended release embodiments accordingly.

In certain embodiments, the pharmacokinetics of the auris compatible composition or devices described herein are determined by injecting the composition or device on or near the round window membrane of a test animal (including by way of example, a guinea pig or a chinchilla). At a determined period of time (e.g., 6 hours, 12 hours, 1 day, 2 days, 3 days, 4 days, 5 days, 6 days, and 7 days for testing the pharmacokinetics of a composition or device over a 1 week period), the test animal is euthanized and a 5 mL sample of the perilymph fluid is tested. The inner ear removed and tested for the presence of the active agent. As needed, the level of an active agent is measured in other organs. In addition, the systemic level of the active agent is measured by withdrawing a blood sample from the test animal. In order to determine whether the composition or device impedes hearing, the hearing of the test animal is optionally tested.

Alternatively, an inner ear is provided (as removed from a test animal) and the migration of the active agent is measured. As yet another alternative, an in vitro model of a round window membrane is provided and the migration of the active agent is measured.

As described herein, composition or devices comprising micronized active agents provide extended release over a longer period of time compared to composition or devices comprising non-micronized active agents. In some instances, the micronized active agent provides a steady supply (e.g., +/−20%) of active agent via slow degradation and serves as a depot for the active agent; such a depot effect increases residence time of the active agent in the ear. In specific embodiments, selection of an appropriate particle size of the active agent (e.g., micronized active agent) in combination with the amount of gelling agent in the composition or device provides tunable extended release characteristics that allow for release of an active agent over a period of hours, days, weeks or months.

In some embodiments, the viscosity of a composition or device described herein is designed to provide a suitable rate of release from an otic compatible gel. In some embodiments, the concentration of a thickening agent (e.g., gelling components such as polyoxyethylene-polyoxypropylene copolymers) allows for a tunable mean dissolution time (MDT). The MDT is inversely proportional to the release rate of an active agent from a composition or device described herein. Experimentally, the released active agent is optionally fitted to the Korsmeyer-Peppas equation

$$\frac{Q}{{Q}_{α}} = {kt}^{n} + b$$

where Q is the amount of active agent released at time t, Q_{α} is the overall released amount of active agent, k is a release constant of the nth order, n is a dimensionless number related to the dissolution mechanism and b is the axis intercept, characterizing the initial burst release mechanism wherein n=1 characterizes an erosion controlled mechanism. The mean dissolution time (MDT) is the sum of different periods of time the drug molecules stay in the matrix before release, divided by the total number of molecules and is optionally calculated by:

$$MDT = \frac{{nk}^{- 1 / n}}{n + 1}$$

For example, a linear relationship between the mean dissolution time (MDT) of a composition or device and the concentration of the gelling agent (e.g., poloxamer) indicates that the active agent is released due to the erosion of the polymer gel (e.g., poloxamer) and not via diffusion. In another example, a non-linear relationship indicates release of active agent via a combination of diffusion and/or polymer gel degradation. In another example, a faster gel elimination time course of a composition or device (a faster release of active agent) indicates lower mean dissolution time (MDT). The concentration of gelling components and/or active agent in a composition or device are tested to determine suitable parameters for MDT. In some embodiments, injection volumes are also tested to determine suitable parameters for preclinical and clinical studies. The gel strength and concentration of the active agent affects release kinetics of an active agent from the composition or device. At low poloxamer concentration, elimination rate is accelerated (MDT is lower). An increase in active agent concentration in the composition or device prolongs residence time and/or MDT of the active agent in the ear.

In some embodiments, the MDT for poloxamer from a composition or device described herein is at least 6 hours. In some embodiments, the MDT for poloxamer from a composition or device described herein is at least 10 hours.

In some embodiments, the MDT for an active agent from a composition or device described herein is from about 30 hours to about 48 hours. In some embodiments, the MDT for an active agent from a composition or device described herein is from about 30 hours to about 96 hours. In some embodiments, the MDT for an active agent from a composition or device described herein is from about 30 hours to about 1 week. In some embodiments, the MDT for a composition or device described herein is from about 1 week to about 6 weeks.

In certain embodiments, a controlled release otic composition or device described herein increases the exposure of an active agent and increases the Area Under the Curve (AUC) in otic fluids (e.g., endolymph and/or perilymph) by about 30%, about 40%, about 50%, about 60%, about 70%, about 80% or about 90% compared to a composition or device that is not a controlled release otic composition or device. In certain embodiments, a controlled release otic composition or device described herein increases the exposure time of an active agent and decreases the Cₘₐₓ in otic fluids (e.g., endolymph and/or perilymph) by about 40%, about 30%, about 20%, or about 10%, compared to a composition or device that is not a controlled release otic composition or device. In certain embodiments, a controlled release otic composition or device described herein alters (e.g. reduces) the ratio of Cₘₐₓ to Cₘᵢₙ compared to a composition or device that is not a controlled release otic composition or device. In certain embodiments, a controlled release otic composition or device described herein increases the exposure of an active agent and increases the length of time that the concentration of an active agent is above Cₘᵢₙ by about 30%, about 40%, about 50%, about 60%, about 70%, about 80% or about 90% compared to a composition or device that is not a controlled release otic composition or device. In certain instances, controlled release composition or devices described herein delay the time to Cₘₐₓ. In certain instances, the controlled steady release of a drug prolongs the time the concentration of the drug will stay above the Cₘᵢₙ. In some embodiments, the composition or devices described herein prolong the residence time of a drug in the inner ear and provide a stable drug exposure profile. In some instances, an increase in concentration of an active agent in the composition or device saturates the clearance process and allows for a more rapid and stable steady state to be reached.

In certain instances, once drug exposure (e.g., concentration in the endolymph or perilymph) of a drug reaches steady state, the concentration of the drug in the endolymph or perilymph stays at or about the therapeutic dose for an extended period of time (e.g., one day, 2 days, 3 days, 4 days, 5 days, 6 days, or 1 week, 3 weeks, 6 weeks, 2 months). In some embodiments, the steady state concentration of active agent released from a controlled release composition or device described herein is about 20 to about 50 times the steady state concentration of an active agent released from a composition or device that is not a controlled release composition or device.

The release of an active agent from a composition or device disclosed herein is optionally tunable to the desired release characteristics. In some embodiments, a composition or device disclosed herein is a solution that is substantially free of gelling components. In such instances, the composition or device provides essentially immediate release of an active agent. In some of such embodiments, the composition or device is useful in perfusion of otic structures, e.g., during surgery.

In some embodiments, a composition or device disclosed herein is a solution that is substantially free of gelling components and comprises micronized active agent. In some of such embodiments, the composition or device provides intermediate release of an active agent from about 2 day to about 4 days.

In some embodiments, a composition or device disclosed herein comprises a gelling agent (e.g., poloxamer 407) and provides release of an active agent over a period of from about 1 day to about 3 days. In some embodiments, a composition or device disclosed herein comprises a gelling agent (e.g., poloxamer 407) and provides release of an active agent over a period of from about 1 day to about 5 days. In some embodiments, a composition or device disclosed herein comprises a gelling agent (e.g., poloxamer 407) and provides release of an active agent over a period of from about 2 days to about 7 days.

In some embodiments, a composition or device disclosed herein comprises a gelling agent (e.g., poloxamer 407) in combination with micronized active agent and provides extended sustained release. In some embodiments, a composition or device disclosed herein comprises (a) about 14-17% of a gelling agent (e.g., poloxamer 407) and (b) a micronized active agent; and provides extended sustained release over a period of from about 1 week to about 3 weeks. In some embodiments, a composition or device disclosed herein comprises (a) about 16% of a gelling agent (e.g., poloxamer 407) and (b) a micronized active agent; and provides extended sustained release over a period of from about 3 weeks. In some embodiments, a composition or device disclosed herein comprises (a) about 18-21% of a gelling agent (e.g., poloxamer 407) and (b) a micronized active agent; and provides extended sustained release over a period of from about 3 weeks to about 6 weeks. In some embodiments, a composition or device disclosed herein comprises (a) about 20% of a gelling agent (e.g., poloxamer 407) and (b) a micronized active agent; and provides extended sustained release over a period of from about 6 weeks. In some embodiments, the amount of gelling agent in a composition or device, and the particle size of an active agent are tunable to the desired release profile of an active agent from the composition or device.

In specific embodiments, composition or devices comprising micronized active agents provide extended release over a longer period of time compared to composition or devices comprising non-micronized active agents. In specific embodiments, selection of an appropriate particle size of the active agent (e.g., micronized active agent) in combination with the amount of gelling agent in the composition or device provides tunable extended release characteristics that allow for release of an active agent over a period of hours, days, weeks or months.

### Kits/Articles of Manufacture

The disclosure also provides kits for preventing, treating or ameliorating the symptoms of a disease or disorder in a mammal. Such kits generally will comprise one or more of the anti-apoptotic agent or pro-apoptotic agent controlled-release compositions or devices disclosed herein, and instructions for using the kit. The disclosure also contemplates the use of one or more of the anti-apoptotic agent or pro-apoptotic agent controlled-release compositions, in the manufacture of medicaments for treating, abating, reducing, or ameliorating the symptoms of a disease, dysfunction, or disorder in a mammal, such as a human that has, is suspected of having, or at risk for developing an inner ear disorder.

In some embodiments, kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In other embodiments, the containers are formed from a variety of materials such as glass or plastic.

The articles of manufacture provided herein contain packaging materials. Packaging materials for use in packaging pharmaceutical products are also presented herein. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected composition and intended mode of administration and treatment. A wide array of an anti-apoptotic agent or pro-apoptotic agent compositions provided herein are contemplated as are a variety of treatments for any disease, disorder, or condition that would benefit by controlled-release administration of an anti-apoptotic agent or pro-apoptotic agent to the inner ear.

In some embodiments, a kit includes one or more additional containers, each with one or more of various materials (such as reagents, optionally in concentrated form, and/or devices) desirable from a commercial and user standpoint for use of a composition described herein. Non-limiting examples of such materials include, but not limited to, buffers, diluents, filters, needles, syringes; carrier, package, container, vial and/or tube labels listing contents and/or instructions for use and package inserts with instructions for use. A set of instructions is optionally included. In a further embodiment, a label is on or associated with the container. In yet a further embodiment, a label is on a container when letters, numbers or other characters forming the label are attached, molded or etched into the container itself; a label is associated with a container when it is present within a receptacle or carrier that also holds the container, e.g., as a package insert. In other embodiments a label is used to indicate that the contents are to be used for a specific therapeutic application. In yet another embodiment, a label also indicates directions for use of the contents, such as in the methods described herein.

In certain embodiments, the pharmaceutical compositions are presented in a pack or dispenser device that contains one or more unit dosage forms containing a compound provided herein. In another embodiment, the pack for example contains metal or plastic foil, such as a blister pack. In a further embodiment, the pack or dispenser device is accompanied by instructions for administration. In yet a further embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. In another embodiment, such notice, for example, is the labeling approved by the U.S. Food and Drug Administration for prescription drugs, or the approved product insert. In yet another embodiment, compositions containing a compound provided herein formulated in a compatible pharmaceutical carrier are also prepared, placed in an appropriate container, and labeled for treatment of an indicated condition.

### EXAMPLES

### Example 1

### Preparation of a Thermoreversible Gel XIAP Formulation

| | Quantity (mg/g of |
|---|---|
| Ingredient | formulation) |
| XIAP | 21.0 |
| methylparaben | 2.1 |
| Hypromellose | 21.0 |
| Poloxamer 407 | 378 |
| TRIS HCl buffer (0.1 M) | 1677.9 |

A 10-g batch of gel formulation containing 1.0% of XIAP is prepared by first suspending Poloxamer 407 (BASF Corp.) in TRIS HCl buffer (0.1 M). The Poloxamer 407 and TRIS are mixed under agitation overnight at 4° C. to ensure complete dissolution of the Poloxamer 407 in the TRIS. The hypromellose, methylparaben and additional TRIS HCl buffer (0.1 M) is added. The composition is stirred until dissolution is observed. A solution of XIAP is added and the composition is mixed until a homogenous gel is produced. The mixture is maintained below room temperature until use.

### Example 2

### Preparation of a Mucoadhesive, Thermoreversible Gel AM-111 Formulation

| | Quantity (mg/g of |
|---|---|
| Ingredient | formulation) |
| AM-111 | 25.5 |
| methylparaben | 2.55 |
| Hypromellose | 25.5 |
| Carbopol 934P | 5.1 |
| Poloxamer 407 | 459 |
| TRIS HCl buffer (0.1 M) | 2032.35 |

A 10-g batch of mucoadhesive gel formulation containing 1.0% of AM-111 is prepared by first suspending Poloxamer 407 (BASF Corp.) and Carbopol 934P in TRIS HCl buffer (0.1 M). The Poloxamer 407, Carbopol 934P and TRIS are mixed under agitation overnight at 4° C. to ensure complete dissolution of the Poloxamer 407 and Carbopol 934P in the TRIS. The hypromellose, methylparaben and additional TRIS HCl buffer (0.1 M) is added. The composition is stirred until dissolution is observed. The AM-111 solution is added and the composition is mixed until a homogenous gel is produced. The mixture is maintained below room temperature until use.

### Example 3

### Preparation of a Mucoadhesive, Thermoreversible Gel SB-203580 Formulation

| | Quantity (mg/g of |
|---|---|
| Ingredient | formulation) |
| SB-203580 | 25.5 |
| methylparaben | 2.55 |
| Hypromellose | 25.5 |
| Carbopol 934P | 5.1 |
| Poloxamer 407 | 459 |
| TRIS HCl buffer (0.1 M) | 2032.35 |

SB-203580 is supplied as a solid. It is rehydrated in water to a final molarity of 10 mM.

A 10-g batch of mucoadhesive gel formulation containing 1.0% of SB-203580 is prepared by first suspending Poloxamer 407 (BASF Corp.) and Carbopol 934P in TRIS HCl buffer (0.1 M). The Poloxamer 407, Carbopol 934P and TRIS are mixed under agitation overnight at 4° C. to ensure complete dissolution of the Poloxamer 407 and Carbopol 934P in the TRIS. The hypromellose, methylparaben and additional TRIS HCl buffer (0.1 M) is added. The composition is stirred until dissolution is observed. The SB-203580 solution is added and the composition is mixed until a homogenous gel is produced. The mixture is maintained below room temperature until use.

### Example 4

### Preparation of a Hydrogel-Based Leupeptine Formulation

| | Quantity (mg/g of |
|---|---|
| Ingredient | formulation) |
| Leupeptine | 10.0 |
| paraffin oil | 200.0 |
| trihydroxystearate | 10.0 |
| cetyl dimethicon copolyol | 30.0 |
| water | qs ad 1000 |
| phosphate buffer pH 7.4 | qs pH 7.4 |

The cream-type formulation is first prepared by gently mixing leupeptine with water until the leupeptine is dissolved. Then, the oil base is prepared by mixing paraffin oil, trihydroxystearate and cetyl dimethicon copolyol at temperatures up to 60° C. The oil base is cooled to room temperature and the leupeptine solution is added. The two phases are mixed until a homogenous, monophasic hydrogel is formed.

### Example 5

### Preparation of a Gel Minocycline Formulation

| | Quantity (mg/g of |
|---|---|
| Ingredient | formulation) |
| Minocycline | 16 |
| chitosan | 8 |
| Glycerophosphate disodium | 32 |
| water | 336 |

A 5 ml solution of acetic acid is titrated to a pH of about 4.0. The chitosan is added to achieve a pH of about 5.5. The minocycline is then dissolved in the chitosan solution. This solution is sterilized by filtration. A 5 ml aqueous solution of glycerophosphate disodium is also prepared and sterilized. The two solutions are mixed and within 2 h at 37° C., the desired gel is formed.

### Example 6

### Application of an Enhanced Viscosity SB-203580 Formulation onto the Round Window Membrane

A formulation according to Example 3 is prepared and loaded into 5 ml siliconized glass syringes attached to a 15-gauge luer lock disposable needle. SB-203580 is topically applied to the tympanic membrane, and a small incision made to allow visualization into the middle ear cavity. The needle tip is guided into place over the round window membrane, and the SB-203580 formulation applied directly onto the round-window membrane.

### Example 7

### Effect of pH on Degradation Products for Autoclaved 17% Poloxamer 407NF/2% Otic Agent in PBS Buffer

A stock solution of a 17% poloxamer 407/2% otic agent is prepared by dissolving 351.4 mg of sodium chloride (Fisher Scientific), 302.1 mg of sodium phosphate dibasic anhydrous (Fisher Scientific), 122.1 mg of sodium phosphate monobasic anhydrous (Fisher Scientific) and an appropriate amount of an otic agent with 79.3 g of sterile filtered DI water. The solution is cooled down in a ice chilled water bath and then 17.05 g of poloxamer 407NF (SPECTRUM CHEMICALS) is sprinkled into the cold solution while mixing. The mixture is further mixed until the poloxamer is completely dissolved. The pH for this solution is measured.

17% poloxamer 407/2% otic agent in PBS pH of 5.3. Take an aliquot (approximately 30 mL) of the above solution and adjust the pH to 5.3 by the addition of 1 M HCl.

17% poloxamer 407/2% otic agent in PBS pH of 8.0. Take an aliquot (approximately 30 mL) of the above stock solution and adjust the pH to 8.0 by the addition of 1 M NaOH.

A PBS buffer (pH 7.3) is prepared by dissolving 805.5 mg of sodium chloride (Fisher Scientific), 606 mg of sodium phosphate dibasic anhydrous (Fisher Scientific), 247 mg of sodium phosphate monobasic anhydrous (Fisher Scientific), then QS to 200 g with sterile filtered DI water.

A 2% solution of an otic agent in PBS pH 7.3 is prepared by dissolving an appropriate amount of the otic agent in the PBS buffer and QS to 10 g with PBS buffer.

One mL samples are individually placed in 3 mL screw cap glass vials (with rubber lining) and closed tightly. The vials are placed in a Market Forge-sterilmatic autoclave (settings, slow liquids) and sterilized at 250° F. for 15 minutes. After the autoclave the samples are left to cool down to room temperature and then placed in refrigerator. The samples are homogenized by mixing the vials while cold.

Appearance (e.g., discoloration and/or precipitation) is observed and recorded. HPLC analysis is performed using an Agilent 1200 equipped with a Luna C18(2) 3 μm, 100 Å, 250×4.6 mm column) using a 30-80 acetonitrile gradient (1-10 min) of (water-acetonitrile mixture containing 0.05% TFA), for a total run of 15 minutes. Samples are diluted by taking 30 μL of sample and dissolved with 1.5 mL of a 1:1 acetonitrile water mixture. Purity of the otic agent in the autoclaved samples is recorded.

In general the composition should not have any individual impurity (e.g., degradation product of otic agent) of more than 2% and more preferably not more than one percent. In addition, the composition should not precipitate during storage or change in color after manufacturing and storage.

Compositions comprising SB-203580, PD 169316, SB 202190, RWY 67657, AM-111, micronized SB-203580, or micronized AM-111, prepared according to the procedure in Example 6, are tested using the above procedure to determine the effect of pH on degradation during the autoclaving step.

### Example 8

### Effect of Autoclaving on the Release Profile and Viscosity of a 17% Poloxamer 407NF/2% Otic Agent in PBS

An aliquot of the sample from example 6 (autoclaved and not autoclaved) is evaluated for release profile and viscosity measurement to evaluate the impact of heat sterilization on the properties of the gel.

Dissolution is performed at 37° C. in snapwells (6.5 mm diameter polycarbonate membrane with a pore size of 0.4 μm). 0.2 mL of gel is placed into snapwell and left to harden, then 0.5 mL is placed into reservoir and shaken using a Labline orbit shaker at 70 rpm. Samples are taken every hour (0.1 mL withdrawn and replace with warm buffer). Samples are analyzed for poloxamer concentration by UV at 624 nm using the cobalt thiocyanate method, against an external calibration standard curve. In brief, 20 μL of the sample is mixed with 1980 μL of a 15 mM cobalt thiocyanate solution and absorbance measured at 625 nm, using a Evolution 160 U/V is spectrophotometer (Thermo Scientific).

The released otic agent is fitted to the Korsmeyer-Peppas equation

$$\frac{Q}{{Q}_{α}} = {kt}^{n} + b$$

where Q is the amount of otic agent released at time t, Q_{α} is the overall released amount of otic agent, k is a release constant of the nth order, n is a dimensionless number related to the dissolution mechanism and b is the axis intercept, characterizing the initial burst release mechanism wherein n=1 characterizes an erosion controlled mechanism. The mean dissolution time (MDT) is the sum of different periods of time the drug molecules stay in the matrix before release, divided by the total number of molecules and is calculated by:

$$MDT = \frac{{nk}^{- 1 / n}}{n + 1}$$

Viscosity measurements are performed using a Brookfield viscometer RVDV-II+P with a CPE-51 spindle rotated at 0.08 rpm (shear rate of 0.31 s⁻¹), equipped with a water jacketed temperature control unit (temperature ramped from 15-34° C. at 1.6° C./min). Tgel is defined as the inflection point of the curve where the increase in viscosity occurs due to the sol-gel transition.

Compositions comprising SB-203580, PD 169316, SB 202190, RWY 67657, AM-111, micronized SB-203580, or micronized AM-111 prepared according to the procedure in Example 6, are tested using the procedure described above to determine Tgel.

### Example 9

### Effect of Addition of a Secondary Polymer on the Degradation Products and Viscosity of a Composition Containing 2% Otic Agent and 17% Poloxamer 407NF after Heat Sterilization (Autoclaving)

Solution A. A solution of pH 7.0 comprising sodium carboxymethylcellulose (CMC) in PBS buffer is prepared by dissolving 178.35 mg of sodium chloride (Fisher Scientific), 300.5 mg of sodium phosphate dibasic anhydrous (Fisher Scientific), 126.6 mg of sodium phosphate monobasic anhydrous (Fisher Scientific) dissolved with 78.4 of sterile filtered DI water, then 1 g of Blanose 7M65 CMC (Hercules, viscosity of 5450 cP @ 2%) is sprinkled into the buffer solution and heated to aid dissolution, and the solution is then cooled down.

A solution of pH 7.0 comprising 17% poloxamer 407NF/1% CMC/2% otic agent in PBS buffer is made by cooling down 8.1 g of solution A in a ice chilled water bath and then adding an appropriate amount of an otic agent followed by mixing. 1.74 g of poloxamer 407NF (Spectrum Chemicals) is sprinkled into the cold solution while mixing. The mixture is further mixed until all the poloxamer is completely dissolved.

Two mL of the above sample is placed in a 3 mL screw cap glass vial (with rubber lining) and closed tightly. The vial is placed in a Market Forge-sterilmatic autoclave (settings, slow liquids) and sterilized at 250° F. for 25 minutes. After autoclaving the sample is left to cool down to room temperature and then placed in refrigerator. The sample is homogenized by mixing while the vials are cold.

Precipitation or discoloration are observed after autoclaving. HPLC analysis is performed using an Agilent 1200 equipped with a Luna C18(2) 3 μm, 100 Å, 250×4.6 mm column) using a 30-80 acetonitrile gradient (1-10 min) of (water-acetonitrile mixture containing 0.05% TFA), for a total run of 15 minutes. Samples are diluted by taking 30 μL of sample and dissolving with 1.5 mL of a 1:1 acetonitrile water mixture. Purity of the otic agent in the autoclaved samples is recorded.

Viscosity measurements are performed using a Brookfield viscometer RVDV-II+P with a CPE-51 spindle rotated at 0.08 rpm (shear rate of 0.31 s⁻¹), equipped with a water jacketed temperature control unit (temperature ramped from 15-34° C. at 1.6° C./min). Tgel is defined as the inflection point of the curve where the increase in viscosity occurs due to the sol-gel transition.

Dissolution is performed at 37° C. for the non-autoclaved sample in snapwells (6.5 mm diameter polycarbonate membrane with a pore size of 0.4 μm), 0.2 mL of gel is placed into snapwell and left to harden, then 0.5 mL is placed into reservoir and shaken using a Labline orbit shaker at 70 rpm. Samples are taken every hour (0.1 mL withdrawn and replaced with warm buffer). Samples are analyzed for otic agent concentration by UV at 245 nm, against an external calibration standard curve.

Compositions comprising SB-203580, PD 169316, SB 202190, RWY 67657, AM-111, micronized SB-203580, or micronized AM-111 are tested using the above procedure to determine the effect addition of a secondary polymer on the degradation products and viscosity of a composition containing 2% otic agent and 17% poloxamer 407NF after heat sterilization (autoclaving).

### Example 10

### Effect of Buffer Type on the Degradation Products for Compositions Containing Poloxamer 407NF after Heat Sterilization (Autoclaving)

A TRIS buffer is made by dissolving 377.8 mg of sodium chloride (Fisher Scientific), and 602.9 mg of Tromethamine (Sigma Chemical Co.) then QS to 100 g with sterile filtered DI water, pH is adjusted to 7.4 with 1M HCl.

Stock solution containing 25% Poloxamer 407 solution in TRIS buffer:

Weigh 45 g of TRIS buffer, chill in an ice chilled bath then sprinkle into the buffer, while mixing, 15 g of poloxamer 407 NF (Spectrum Chemicals). The mixture is further mixed until all the poloxamer is completely dissolved.

A series of compositions is prepared with the above stock solution. An appropriate amount of otic agent (or salt or prodrug thereof) and/or otic agent as micronized/coated/liposomal particles (or salt or prodrug thereof) is used for all experiments.

Stock solution (pH 7.3) containing 25% Poloxamer 407 solution in PBS buffer:

PBS buffer is prepared by dissolving 704 mg of sodium chloride (Fisher Scientific), 601.2 mg of sodium phosphate dibasic anhydrous (Fisher Scientific), 242.7 mg of sodium phosphate monobasic anhydrous (Fisher Scientific) with 140.4 g of sterile filtered DI water. The solution is cooled down in an ice chilled water bath and then 50 g of poloxamer 407NF (SPECTRUM CHEMICALS) is sprinkled into the cold solution while mixing. The mixture is further mixed until the poloxamer is completely dissolved.

A series of compositions is prepared with the above stock solution. An appropriate amount of otic agent (or salt or prodrug thereof) and/or otic agent as micronized/coated/liposomal particles (or salt or prodrug thereof) is used for all experiments.

Tables 2 and 3 list samples prepared using the procedures described above. An appropriate amount of otic agent is added to each sample to provide a final concentration of 2% otic agent in the sample.

TABLE 2
Preparation of samples containing TRIS buffer

| | | 25% Stock | TRIS |
|---|---|---|---|
| Sample | pH | Solution (g) | Buffer (g) |
| 20% P407/2 otic agent/TRIS | 7.45 | 8.01 | 1.82 |
| 18% P407/2 otic agent/TRIS | 7.45 | 7.22 | 2.61 |
| 16% P407/2 otic agent/TRIS | 7.45 | 6.47 | 3.42 |
| 18% P4072 otic agent/TRIS | 7.4 | 7.18 | 2.64 |
| 4% otic agent/TRIS | 7.5 | — | 9.7 |
| 2% otic agent/TRIS | 7.43 | — | 5 |
| 1% otic agent/TRIS | 7.35 | — | 5 |
| 2% otic agent/TRIS (suspension) | 7.4 | — | 4.9 |

TABLE 3
Preparation of samples containing PBS buffer (pH of 7.3)

| | 25% Stock Solution | PBS |
|---|---|---|
| Sample | in PBS (g) | Buffer (g) |
| 20% P407/2 otic agent/PBS | 8.03 | 1.82 |
| 18% P407/2 otic agent/PBS | 7.1 | 2.63 |
| 16% P407/2 otic agent/PBS | 6.45 | 3.44 |
| 18% P407/2 otic agent/PBS | — | 2.63 |
| 2% otic agent/PBS | — | 4.9 |

One mL samples are individually placed in 3 mL screw cap glass vials (with rubber lining) and closed tightly. The vials are placed in a Market Forge-sterilmatic autoclave (setting, slow liquids) and sterilized at 250° F. for 25 minutes. After the autoclaving the samples are left to cool down to room temperature. The vials are placed in the refrigerator and mixed while cold to homogenize the samples.

HPLC analysis is performed using an Agilent 1200 equipped with a Luna C18(2) 3 μm, 100 Å, 250×4.6 mm column) using a 30-80 acetonitrile gradient (1-10 min) of (water-acetonitrile mixture containing 0.05% TFA), for a total run of 15 minutes. Samples are diluted by taking 30 μL of sample and dissolving with 1.5 mL of a 1:1 acetonitrile water mixture. Purity of the otic agent in the autoclaved samples is recorded. The stability of compositions in TRIS and PBS buffers is compared.

Viscosity measurements are performed using a Brookfield viscometer RVDV-II+P with a CPE-51 spindle rotated at 0.08 rpm (shear rate of 0.31 s⁻¹), equipped with a water jacketed temperature control unit (temperature ramped from 15-34° C. at 1.6° C./min). Tgel is defined as the inflection point of the curve where the increase in viscosity occurs due to the sol-gel transition. Only compositions that show no change after autoclaving are analyzed.

Compositions comprising SB-203580, PD 169316, SB 202190, RWY 67657, AM-111, micronized SB-203580, or micronized AM-111 are tested using the above procedure to determine the effect addition of a secondary polymer on the degradation products and viscosity of a composition containing 2% otic agent and 17% poloxamer 407NF after heat sterilization (autoclaving). Stability of compositions containing micronized otic agent is compared to non-micronized otic agent composition counterparts.

### Example 11

### Pulsed Release Otic Compositions

Diazepam is used to prepare a pulsed release otic agent composition using the procedures described herein. A 17% poloxamer solution is prepared by dissolving 351.4 mg of sodium chloride (Fisher Scientific), 302.1 mg of sodium phosphate dibasic anhydrous (Fisher Scientific), 122.1 mg of sodium phosphate monobasic anhydrous (Fisher Scientific) and an appropriate amount of an otic agent with 79.3 g of sterile filtered DI water. The solution is cooled down in a ice chilled water bath and then 17.05 g of poloxamer 407NF (SPECTRUM CHEMICALS) is sprinkled into the cold solution while mixing. The mixture is further mixed until the poloxamer is completely dissolved. The pH for this solution is measured. 20% of the delivered dose of diazepam is solubilized in the 17% poloxamer solution with the aid of beta-cyclodextrins. The remaining 80% of the otic agent is then added to the mixture and the final composition is prepared using any procedure described herein.

Pulsed release compositions comprising SB-203580, PD 169316, SB 202190, RWY 67657, AM-111, micronized SB-203580, or micronized AM-111 prepared according to the procedures and examples described herein, are tested using procedures described herein to determine pulse release profiles.

### Example 12

### Preparation of A 17% Poloxamer 407/2% Otic Agent/78 Ppm Evans Blue in PBS

A Stock solution of Evans Blue (5.9 mg/mL) in PBS buffer is prepared by dissolving 5.9 mg of Evans Blue (Sigma Chemical Co) with 1 mL of PBS buffer. PBS buffer is prepared by dissolving 704 mg of sodium chloride (Fisher Scientific), 601.2 mg of sodium phosphate dibasic anhydrous (Fisher Scientific), 242.7 mg of sodium phosphate monobasic anhydrous (Fisher Scientific) with 140.4 g of sterile filtered DI water.

A Stock solution containing 25% Poloxamer 407 solution in PBS buffer (as in Example 9) is used in this study. An appropriate amount of an otic agent is added to the 25% Poloxamer 407 solution stock solution to prepare compositions comprising 2% of an otic agent (Table 4).

TABLE 4
Preparation of poloxamer 407 samples containing Evans Blue

| | 25% P407 in | PBS | Evans Blue |
|---|---|---|---|
| Sample ID | PBS (g) | Buffer (g) | Solution (μL) |
| 17% P407/2 otic agent/EB | 13.6 | 6 | 265 |
| 20% P407/2 otic agent/EB | 16.019 | 3.62 | 265 |
| 25% P407/2 otic agent/EB | 19.63 | — | 265 |

Compositions comprising SB-203580, PD 169316, SB 202190, RWY 67657, AM-111, micronized SB-203580, or micronized AM-111 are prepared according to the procedures in Example 12 and are sterile filtered through 0.22 μm PVDF syringe filters (Millipore corporation), and autoclaved.

The above compositions are dosed to guinea pigs in the middle ear by procedures described herein and the ability of compositions to gel upon contact and the location of the gel is identified after dosing and at 24 hours after dosing.

### Example 13

### Terminal Sterilization of Poloxamer 407 Compositions with and without a Visualization Dye

17% poloxamer 407/2% otic agent/in phosphate buffer, pH 7.3: Dissolve 709 mg of sodium chloride (Fisher Scientific), 742 mg of sodium phosphate dibasic dehydrate USP (Fisher Scientific), 251.1 mg of sodium phosphate monobasic monohydrate USP (Fisher Scientific) and an appropriate amount of an otic agent with 158.1 g of sterile filtered DI water. The solution is cooled down in an ice chilled water bath and then 34.13 g of poloxamer 407NF (Spectrum chemicals) is sprinkled into the cold solution while mixing. The mixture is further mixed until the poloxamer is completely dissolved.

17% poloxamer 407/2% otic agent/59 ppm Evans blue in phosphate buffer: Take two mL of the 17% poloxamer 407/2% otic agent/in phosphate buffer solution and add 2 mL of a 5.9 mg/mL Evans blue (Sigma-Aldrich chemical Co) solution in PBS buffer.

25% poloxamer 407/2% otic agent/in phosphate buffer: Dissolve 330.5 mg of sodium chloride (Fisher Scientific), 334.5 mg of sodium phosphate dibasic dehydrate USP (Fisher Scientific), 125.9 mg of sodium phosphate monobasic monohydrate USP (Fisher Scientific) and an appropriate amount of an otic agent with 70.5 g of sterile filtered DI water.

The solution is cooled down in an ice chilled water bath and then 25.1 g of poloxamer 407NF (Spectrum chemicals) is sprinkled into the cold solution while mixing. The mixture is further mixed until the poloxamer is completely dissolved.

25% poloxamer 407/2% otic agent/59 ppm Evans blue in phosphate buffer: Take two mL of the 25% poloxamer 407/2% otic agent/in phosphate buffer solution and add 2 mL of a 5.9 mg/mL Evans blue (Sigma-Aldrich chemical Co) solution in PBS buffer.

Place 2 mL of composition into a 2 mL glass vial (Wheaton serum glass vial) and seal with 13 mm butyl str (kimble stoppers) and crimp with a 13 mm aluminum seal. The vials are placed in a Market Forge-sterilmatic autoclave (settings, slow liquids) and sterilized at 250° F. for 25 minutes. After the autoclaving the samples are left to cool down to room temperature and then placed in refrigeration. The vials are placed in the refrigerator and mixed while cold to homogenize the samples. Sample discoloration or precipitation after autoclaving is recorded.

HPLC analysis is performed using an Agilent 1200 equipped with a Luna C18(2) 3 μm, 100 Å, 250×4.6 mm column) using a 30-95 methanol:acetate buffer pH 4 gradient (1-6 min), then isocratic for 11 minutes, for a total run of 22 minutes. Samples are diluted by taking 30 μL of sample and dissolved with 0.97 mL of water. The main peaks are recorded in the table below. Purity before autoclaving is always greater than 99% using this method.

Viscosity measurements are performed using a Brookfield viscometer RVDV-II+P with a CPE-51 spindle rotated at 0.08 rpm (shear rate of 0.31 s⁻¹), equipped with a water jacketed temperature control unit (temperature ramped from 15-34° C. at 1.6° C./min). Tgel is defined as the inflection point of the curve where the increase in viscosity occurs due to the sol-gel transition.

Compositions comprising SB-203580, PD 169316, SB 202190, RWY 67657, AM-111, micronized SB-203580, or micronized AM-111 prepared according to the procedure in Example 11, are tested using the above procedures to determine stability of the compositions.

### Example 14

### In vitro Comparison of Release Profile

Dissolution is performed at 37° C. in snapwells (6.5 mm diameter polycarbonate membrane with a pore size of 0.4 μm), 0.2 mL of a gel composition described herein is placed into snapwell and left to harden, then 0.5 mL buffer is placed into reservoir and shaken using a Labline orbit shaker at 70 rpm. Samples are taken every hour (0.1 mL withdrawn and replace with warm buffer). Samples are analyzed for otic agent concentration by UV at 245 nm against an external calibration standard curve. Pluronic concentration is analyzed at 624 nm using the cobalt thiocyanate method. Relative rank-order of mean dissolution time (MDT) as a function of % P407 is determined. A linear relationship between the compositions mean dissolution time (MDT) and the P407 concentration indicates that the otic agent is released due to the erosion of the polymer gel (poloxamer) and not via diffusion. A non-linear relationship indicates release of otic agent via a combination of diffusion and/or polymer gel degradation.

Alternatively, samples are analyzed using the method described by Li Xin-Yu paper [Acta Pharmaceutica Sinica 2008, 43(2):208-203] and Rank-order of mean dissolution time (MDT) as a function of % P407 is determined.

Compositions comprising SB-203580, PD 169316, SB 202190, RWY 67657, AM-111, micronized SB-203580, or micronized AM-111 prepared according to the procedures described herein, are tested using the above procedure to determine the release profile of the otic agents.

### Example 15

### In Vitro Comparison of Gelation Temperature

The effect of Poloxamer 188 and an otic agent on the gelation temperature and viscosity of Poloxamer 407 compositions is evaluated with the purpose of manipulating the gelation temperature.

A 25% Poloxamer 407 stock solution in PBS buffer (as in Example 9) and a PBS solution (as in Example 11) are used. Poloxamer 188NF from BASF is used. An appropriate amount of otic agent is added to the solutions described in Table 5 to provide a 2% composition of the otic agent.

TABLE 5
Preparation of samples containing poloxamer 407/poloxamer 188

| | 25% P407 Stock | Poloxamer 188 | PBS |
|---|---|---|---|
| Sample | Solution (g) | (mg) | Buffer (g) |
| 16% P407/10% P188 | 3.207 | 501 | 1.3036 |
| 17% P407/10% P188 | 3.4089 | 500 | 1.1056 |
| 18% P407/10% P188 | 3.6156 | 502 | 0.9072 |
| 19% P407/10% P188 | 3.8183 | 500 | 0.7050 |
| 20% P407/10% P188 | 4.008 | 501 | 0.5032 |
| 20% P407/5% P188 | 4.01 | 256 | 0.770 |

Mean dissolution time, viscosity and gel temperature of the above compositions are measured using procedures described herein.

An equation is fitted to the data obtained and can be utilized to estimate the gelation temperature of F127/F68 mixtures (for 17-20% F127 and 0-10% F68).

T_{gel}=−1.8(%F127)+1.3(%F68)+53

An equation is fitted to the data obtained and can be utilized to estimate the Mean Dissolution Time (hr) based on the gelation temperature of F127/F68 mixtures (for 17-25% F127 and 0-10% F68), using results obtained in example 13 and 15.

MDT=−0.2(T_{gel})+8

Compositions comprising SB-203580, PD 169316, SB 202190, RWY 67657, AM-111, micronized SB-203580, or micronized AM-111 are prepared by addition of an appropriate amount of otic agents to the solutions described in Table 5. The gel temperature of the compositions is determined using the procedure described above.

### Example 16

### Determination of Temperature Range for Sterile Filtration

The viscosity at low temperatures is measured to help guide the temperature range at that the sterile filtration needs to occur to reduce the possibility of clogging.

Viscosity measurements are performed using a Brookfield viscometer RVDV-II+P with a CPE-40 spindle rotated at 1, 5 and 10 rpm (shear rate of 7.5, 37.5 and 75 s⁻¹), equipped with a water jacketed temperature control unit (temperature ramped from 10-25° C. at 1.6° C./min).

The Tgel of a 17% Pluronic P407 is determined as a function of increasing concentration of otic agent. The increase in Tgel for a 17% pluronic composition is estimated by:

ΔT_{gel}=0.93[% otic agent]

Compositions comprising SB-203580, PD 169316, SB 202190, RWY 67657, AM-111, micronized SB-203580, or micronized AM-111 prepared according to procedures described herein, are tested using the above procedure to determine the temperature range for sterile filtration. The effect of addition of increased amounts of otic agent on the Tgel, and the apparent viscosity of the compositions is recorded.

### Example 17

### Determination of Manufacturing Conditions

TABLE 6
Viscosity of potential compositions at
manufacturing/filtration conditions.

| | Apparent Viscosity^{a} (cP) | | Temperature @ |
|---|---|---|---|
| Sample | 5° C. below Tgel | 20° C. | 100 cP |
| Placebo | 52 cP @ 17° C. | 120 cP | 19° C. |
| 17% P407/2% otic agent | 90 cP @ 18° C. | 147 cP | 18.5° C. |
| 17% P407/6% otic agent | 142 cP @ 22° C. | 105 cP | 19.7° C. |

^{a}Viscosity measured at a shear rate of 37.5 s⁻¹

An 8 liter batch of a 17% P407 placebo is manufactured to evaluate the manufacturing/filtration conditions. The placebo is manufactured by placing 6.4 liters of DI water in a 3 gallon SS pressure vessel, and left to cool down in the refrigerator overnight. The following morning the tank was taken out (water temperature 5° C., RT 18° C.) and 48 g of sodium chloride, 29.6 g of sodium phosphate dibasic dehydrate and 10 g of sodium phosphate monobasic monohydrate is added and dissolved with an overhead mixer (IKA RW20 @ 1720 rpm). Half hour later, once the buffer is dissolved (solution temperature 8° C., RT 18° C.), 1.36 kg of poloxamer 407 NF (spectrum chemicals) is slowly sprinkled into the buffer solution in a 15 minute interval (solution temperature 12° C., RT 18° C.), then speed is increased to 2430 rpm. After an additional one hour mixing, mixing speed is reduced to 1062 rpm (complete dissolution).

The temperature of the room is maintained below 25° C. to retain the temperature of the solution at below 19° C. The temperature of the solution is maintained at below 19° C. up to 3 hours of the initiation of the manufacturing, without the need to chill/cool the container.

Three different Sartoscale (Sartorius Stedim) filters with a surface area of 17.3 cm² are evaluated at 20 psi and 14° C. of solution

1) Sartopore 2, 0.2 μm 5445307HS-FF (PES), flow rate of 16 mL/min

2) Sartobran P, 0.2 μm 5235307HS-FF (cellulose ester), flow rate of 12 mL/min

3) Sartopore 2 XLI, 0.2 μm 54453071S-FF (PES), flow rate of 15 mL/min

Sartopore 2 filter 5441307H4-SS is used, filtration is carried out at the solution temperature using a 0.45, 0.2 μm Sartopore 2150 sterile capsule (Sartorius Stedim) with a surface area of 0.015 m² at a pressure of 16 psi. Flow rate is measured at approximately 100 mL/min at 16 psi, with no change in flow rate while the temperature is maintained in the 6.5-14° C. range. Decreasing pressure and increasing temperature of the solution causes a decrease in flow rate due to an increase in the viscosity of the solution. Discoloration of the solution is monitored during the process.

TABLE 7
Predicted filtration time for a 17% poloxamer 407 placebo at
a solution temperature range of 6.5-14° C. using Sartopore
2, 0.2 μm filters at a pressure of 16 psi of pressure.

| | | Estimated flow rate | Time to filter 8 L |
|---|---|---|---|
| Filter | Size (m²) | (mL/min) | (estimated) |
| Sartopore 2, size 4 | 0.015 | 100 mL/min | 80 min |
| Sartopore 2, size 7 | 0.05 | 330 mL/min | 24 min |
| Sartopore 2, size 8 | 0.1 | 670 mL/min | 12 min |

Viscosity, Tgel and UV/Vis absorption is check before filtration evaluation. Pluronic U/V is spectra are obtained by a Evolution 160 UV/Vis (Thermo Scientific). A peak in the range of 250-300 nm is attributed to BHT stabilizer present in the raw material (poloxamer). Table 8 lists physicochemical properties of the above solutions before and after filtration.

TABLE 8
Physicochemical properties of 17% poloxamer 407
placebo solution before and after filtration

| | | Viscosity^{a} @ | Absorbance @ |
|---|---|---|---|
| Sample | Tgel (° C.) | 19° C. (cP) | 274 nm |
| Before filtration | 22 | 100 | 0.3181 |
| After filtration | 22 | 100 | 0.3081 |

^{a}Viscosity measured at a shear rate of 37.5 s⁻¹

The above process is applicable for manufacture of 17% P407 compositions, and includes temperature analysis of the room conditions. Preferably, a maximum temperature of 19° C. reduces cost of cooling the container during manufacturing. In some instances, a jacketed container is used to further control the temperature of the solution to ease manufacturing concerns.

### Example 18

### In Vitro Release of Otic Agent from an Autoclaved Micronized Sample

17% poloxamer 407/1.5% otic agent in TRIS buffer: 250.8 mg of sodium chloride (Fisher Scientific), and 302.4 mg of Tromethamine (Sigma Chemical Co.) is dissolved in 39.3 g of sterile filtered DI water, pH is adjusted to 7.4 with 1M HCl. 4.9 g of the above solution is used and an appropriate amount of micronized otic agent is suspended and dispersed well. 2 mL of the composition is transferred into a 2 mL glass vial (Wheaton serum glass vial) and sealed with 13 mm butyl styrene (kimble stoppers) and crimped with a 13 mm aluminum seal. The vial is placed in a Market Forge-sterilmatic autoclave (settings, slow liquids) and sterilized at 250° F. for 25 minutes. After the autoclaving the sample is left to cool down to room temperature. The vial is placed in the refrigerator and mixed while cold to homogenize the sample. Sample discoloration or precipitation after autoclaving is recorded.

Dissolution is performed at 37° C. in snapwells (6.5 mm diameter polycarbonate membrane with a pore size of 0.4 μm), 0.2 mL of gel is placed into snapwell and left to harden, then 0.5 mL PBS buffer is placed into reservoir and shaken using a Labline orbit shaker at 70 rpm. Samples are taken every hour [0.1 mL withdrawn and replaced with warm PBS buffer containing 2% PEG-40 hydrogenated castor oil (BASF) to enhance otic agent solubility]. Samples are analyzed for otic agent concentration by UV at 245 nm against an external calibration standard curve. The release rate is compared to other compositions disclosed herein. MDT time is calculated for each sample.

Solubilization of otic agent in the 17% poloxamer system is evaluated by measuring the concentration of the otic agent in the supernatant after centrifuging samples at 15,000 rpm for 10 minutes using an eppendorf centrifuge 5424. Otic agent concentration in the supernatant is measured by UV at 245 nm against an external calibration standard curve.

Compositions comprising SB-203580, PD 169316, SB 202190, RWY 67657, AM-111, micronized SB-203580, or micronized AM-111 prepared according to the procedures described herein, are tested using the above procedures to determine release rate of the otic agent from each composition.

### Example 19

### Release Rate or MDT and Viscosity of Composition Containing Sodium Carboxymethyl Cellulose

17% poloxamer 407/2% otic agent/1% CMC (Hercules Blanose 7M): A sodium carboxymethylcellulose (CMC) solution (pH 7.0) in PBS buffer is prepared by dissolving 205.6 mg of sodium chloride (Fisher Scientific), 372.1 mg of sodium phosphate dibasic dihydrate (Fisher Scientific), 106.2 mg of sodium phosphate monobasic monohydrate (Fisher Scientific) in 78.1 g of sterile filtered DI water. 1 g of Blanose 7M CMC (Hercules, viscosity of 533 cP @ 2%) is sprinkled into the buffer solution and heated to ease solution, solution is then cooled down and 17.08 g poloxamer 407NF (Spectrum Chemicals) is sprinkled into the cold solution while mixing. A composition comprising 17% poloxamer 407NF/1% CMC/2% otic agent in PBS buffer is made adding/dissolving an appropriate amount of otic agent to 9.8 g of the above solution, and mixing until all the otic agent is completely dissolved.

17% poloxamer 407/2% otic agent/0.5% CMC (Blanose 7M65): A sodium carboxymethylcellulose (CMC) solution (pH 7.2) in PBS buffer is prepared by dissolving 257 mg of sodium chloride (Fisher Scientific), 375 mg of sodium phosphate dibasic dihydrate (Fisher Scientific), 108 mg of sodium phosphate monobasic monohydrate (Fisher Scientific) in 78.7 g of sterile filtered DI water. 0.502 g of Blanose 7M65 CMC (Hercules, viscosity of 5450 cP @ 2%) is sprinkled into the buffer solution and heated to ease solution, solution is then cooled down and 17.06 g poloxamer 407NF (Spectrum Chemicals) is sprinkled into the cold solution while mixing. A 17% poloxamer 407NF/1% CMC/2% otic agent solution in PBS buffer is made adding/dissolving an appropriate amount of otic agent to 9.8 g of the above solution, and mixing until the otic agent is completely dissolved.

17% poloxamer 407/2% otic agent/0.5% CMC (Blanose 7H9): A sodium carboxymethylcellulose (CMC) solution (pH 7.3) in PBS buffer is prepared by dissolving 256.5 mg of sodium chloride (Fisher Scientific), 374 mg of sodium phosphate dibasic dihydrate (Fisher Scientific), 107 mg of sodium phosphate monobasic monohydrate (Fisher Scientific) in 78.6 g of sterile filtered DI water, then 0.502 g of Blanose 7H9CMC (Hercules, viscosity of 5600 cP @ 1%) is sprinkled to the buffer solution and heated to ease solution, solution is then cooled down and 17.03 g poloxamer 407NF (Spectrum Chemicals) is sprinkled into the cold solution while mixing. A 17% poloxamer 407NF/1% CMC/2% otic agent solution in PBS buffer is made adding/dissolving an appropriate amount of otic agent to 9.8 of the above solution, and mixing until the otic agent is completely dissolved.

Viscosity measurements are performed using a Brookfield viscometer RVDV-II+P with a CPE-40 spindle rotated at 0.08 rpm (shear rate of 0.6 s⁻¹), equipped with a water jacketed temperature control unit (temperature ramped from 10-34° C. at 1.6° C./min). Tgel is defined as the inflection point of the curve where the increase in viscosity occurs due to the sol-gel transition.

Dissolution is performed at 37° C. in snapwells (6.5 mm diameter polycarbonate membrane with a pore size of 0.4 μm). 0.2 mL of gel is placed into snapwell and left to harden, then 0.5 mL PBS buffer is placed into reservoir and shaken using a Labline orbit shaker at 70 rpm. Samples are taken every hour, 0.1 mL withdrawn and replaced with warm PBS buffer. Samples are analyzed for otic agent concentration by UV at 245 nm against an external calibration standard curve. MDT time is calculated for each of the above compositions.

Compositions comprising SB-203580, PD 169316, SB 202190, RWY 67657, AM-111, micronized SB-203580, or micronized AM-111 prepared according to procedures described above, are tested using the above procedures to determine relationship between release rate and/or mean dissolution time and viscosity of composition containing sodium carboxymethyl cellulose. Any correlation between the mean dissolution time (MDT) and the apparent viscosity (measured at 2° C. below the gelation temperature) is recorded.

### Example 20

### Application of an Enhanced Viscosity Anti-Apoptotic Agent or Pro-Apoptotic Agent Composition onto the Round Window Membrane

A composition according to Example 2 is prepared and loaded into 5 ml siliconized glass syringes attached to a 15-gauge luer lock disposable needle. Lidocaine is topically applied to the tympanic membrane, and a small incision made to allow visualization into the middle ear cavity. The needle tip is guided into place over the round window membrane, and the composition applied directly onto the round-window membrane.

### Example 21

### In Vivo Testing of Intratympanic Injection of an Apoptosis Modulating Composition in a Guinea Pig

A cohort of 21 guinea pigs (Charles River, females weighing 200-300 g) is intratympanically injected with 50 mL of different P407-DSP compositions described herein, containing 0 to 6% of an otic agent. The gel elimination time course for each composition is determined. A faster gel elimination time course of a composition indicates lower mean dissolution time (MDT). Thus the injection volume and the concentration of an anti-apoptotic agent or pro-apoptotic agent in a composition are tested to determine optimal parameters for preclinical and clinical studies.

### Example 22

### In Vivo Extended Release Kinetics

A cohort of 21 guinea pigs (Charles River, females weighing 200-300 g) is intratympanically injected with 50 μL 17% Pluronic F-127 composition buffered at 280 mOsm/kg and containing 1.5% to 4.5% of an anti-apoptotic agent or pro-apoptotic agent by weight of the composition. Animals are dosed on day 1. The release profile for the compositions is determined based on analysis of the perilymph.

### Example 23

### Evaluation of Verapamil in an Acoustic Trauma Mouse Model

Induction of Ototoxicity

Twelve Harlan Sprague-Dawley mice weighing 20 to 24 g are used. Baseline auditory brainstem response (ABR) at 4-20 mHz is measured. The mice are anesthetized and exposed for 30 minutes to a continuous pure tone of 6 kHz at a loudness of 120 dB.

Treatment

The control group (n=10) are administered saline following acoustic trauma. The experimental group (n=10) are administered verapamil (2.0 mg/kg of body weight) following acoustic trauma.

Electrophysiologic Testing

The hearing threshold for the auditory brainstem response threshold (ABR) to click stimuli for each ear of each animal is initially measured and 1 week after the experimental procedure. The animals are placed in a single-walled acoustic booth (Industrial Acoustics Co, Bronx, N.Y., USA) on a heating pad. Subdermal electrodes (Astro-Med, Inc. Grass Instrument Division, West Warwick, R.I., USA) were inserted at the vertex (active electrode), the mastoid (reference), and the hind leg (ground). Click stimuli (0.1 millisecond) are computer generated and delivered to a Beyer DT 48, 200 Ohm speaker fitted with an ear speculum for placement in the external auditory meatus. The recorded ABR is amplified and digitized by a battery-operated preamplifier and input to a Tucker-Davis Technologies ABR recording system that provides computer control of the stimulus, recording, and averaging functions (Tucker Davis Technology, Gainesville, Fla., USA). Successively decreasing amplitude stimuli are presented in 5-dB steps to the animal, and the recorded stimulus-locked activity is averaged (n=512) and displayed. Threshold is defined as the stimulus level between the record with no visibly detectable response and a clearly identifiable response.

### Example 24

### Evaluation of AM-111 in an Acoustic Trauma Mouse Model

Induction of Ototoxicity

Twelve Harlan Sprague-Dawley mice weighing 20 to 24 g are used. Baseline auditory brainstem response (ABR) at 4-20 mHz is measured. The mice are anesthetized and exposed for 30 minutes to a continuous pure tone of 6 kHz at a loudness of 120 dB.

Treatment

The control group (n=10) are administered saline following acoustic trauma. The experimental group (n=10) are administered AM-111 (3.0 mg/kg of body weight) following acoustic trauma.

Electrophysiologic Testing

The hearing threshold for the auditory brainstem response threshold (ABR) to click stimuli for each ear of each animal is initially measured and 1 week after the experimental procedure. The animals are placed in a single-walled acoustic booth (Industrial Acoustics Co, Bronx, N.Y., USA) on a heating pad. Subdermal electrodes (Astro-Med, Inc. Grass Instrument Division, West Warwick, R.I., USA) were inserted at the vertex (active electrode), the mastoid (reference), and the hind leg (ground). Click stimuli (0.1 millisecond) are computer generated and delivered to a Beyer DT 48, 200 Ohm speaker fitted with an ear speculum for placement in the external auditory meatus. The recorded ABR is amplified and digitized by a battery-operated preamplifier and input to a Tucker-Davis Technologies ABR recording system that provides computer control of the stimulus, recording, and averaging functions (Tucker Davis Technology, Gainesville, Fla., USA). Successively decreasing amplitude stimuli are presented in 5-dB steps to the animal, and the recorded stimulus-locked activity is averaged (n=512) and displayed. Threshold is defined as the stimulus level between the record with no visibly detectable response and a clearly identifiable response.

### Example 25

### Application of an Enhanced Viscosity AM-111 Formulation onto the Round Window Membrane

A formulation according to Example 2 is prepared and loaded into 5 ml siliconized glass syringes attached to a 15-gauge luer lock disposable needle. AM-111 is topically applied to the tympanic membrane, and a small incision made to allow visualization into the middle ear cavity. The needle tip is guided into place over the round window membrane, and the AM-111 formulation applied directly onto the round-window membrane.

### Example 26

### Evaluation of Intratympanic Administration of AM-111 on Acute Acoustic Trauma

### Study Objective

The primary objective of this study will be to assess the safety and efficacy of intratympanic (IT) AM-111 treatment for acute acoustic trauma.

### Primary Outcome Measurements

Pure Tone Average (PTA) and Word Recognition as equally weighted endpoints; For Speech Discrimination Scoring, a 50-word monosyllable system will be employed; Greater than 20 dB improvement in PTA or over ALL or SOME of the frequencies where the deficiencies are greater than 30 dB, and/or a 20% or greater improvement in the WDS; In addition to absolute changes, recovery with respect to the contralateral ear will also be determined.
Complete Recovery—recovery to within 5% points of the contralateral speech discrimination score, or within 5 dB of the contralateral PTA.

### Study Design

This will be a multicentre, double-blind, randomized, placebo-controlled, parallel group study comparing intratympanic AM-111 to placebo in the treatment of acute acoustic trauma. Approximately 140 subjects will be enrolled in this study, and randomized (1:1) to 1 of 2 treatment groups based on a randomization sequence.
   a. Subjects in Group I will receive IT AM-111 (1 injection of 2 mg/mL of AM-111 in a thermoreversible gel delivery device; administered once a week for 1 month)
   b. Subjects in Group II will receive a placebo IT injections (1 injection of thermoreversible gel delivery device; administered once a week for 1 month)

### Hearing Assessments

Hearing assessments comprise:
a. Pure Tone Average (500 Hz, 1& 2 kHz; 4, 6 & 8 kHz).
   i. Two PTA values would then be determined: a low frequency value (500 Hz-2 kHz) and a high frequency value (4-8 kHz).
b. Stapedial Reflex
c. Tympanometry & tone decay
d. Speech Recognition Threshold
Before treatment begins hearing loss for each subject will be measured (twice prior to allocation to the study, and once prior to randomization). Hearing assessment at 1, 2, 4 & 8 weeks, 4& 6 months post start of treatment

### Main Criteria for Inclusion

Male or female patients aged between 18 and 75 years

Hearing loss of at least 30 dB developing within 1 month

### Exclusion Criteria

Greater than 10 days of prior oral steroid treatment for any reason within the preceding 30 days

5 or more days of prior oral steroid treatment for acute acoustic trauma within the preceding 14 days

History of fluctuating hearing in either ear.

While preferred embodiments of the present invention have been shown and described herein, such embodiments are provided by way of example only. Various alternatives to the embodiments described herein are optionally employed in practicing the inventions. It is intended that the following claims define the scope of the invention and that methods and structures within the scope of these claims and their equivalents be covered thereby.

We claim:

1. A composition or device for use in the treatment of an otic disease or condition characterized by the apoptosis of a plurality of otic cells, comprising: a therapeutically effective amount of an anti-apoptotic agent having substantially low degradation products; and wherein the delivery device comprises two or more characteristics selected from:
(i) between about 0.1% to about 10% by weight of the anti-apoptotic agent, or pharmaceutically acceptable prodrug or salt thereof,
(ii) between about 14% to about 21% by weight of a polyoxyethylene-polyoxypropylene triblock copolymer of general formula E106 P70 E106;
(iii) sterile water, q.s., buffered to provide a pH between about 5.5 and about 8.0;
(iv) multiparticulate anti-apoptotic agent;
(v) a gelation temperature between about 19° C. to about 42° C.;
(vi) less than about 50 colony forming units (cfu) of microbiological agents per gram of delivery device;
(vii) less than about 5 endotoxin units (EU) per kg of body weight of a subject;
(viii) a mean dissolution time of about 30 hours for the; and
(ix) an apparent viscosity of about 100,000 cP to about 500,000 cP.

2. The pharmaceutical composition or device of claim 1, wherein the composition or device provides a practical osmolarity between about 200 and 400 mOsm/L.

3. The pharmaceutical composition or device of claim 1, wherein the anti-apoptotic agent is released for a period of at least 3 days.

4. The pharmaceutical composition or device of claim 1, wherein the pharmaceutical composition is an auris-acceptable thermoreversible gel.

5. The pharmaceutical composition or device of claim 1, further comprising a dye.

6. The pharmaceutical composition or device of claim 1, wherein the anti-apoptotic agent is in the form of a neutral molecule, a free acid, free base, a salt, a prodrug, or a combination thereof.

7. The pharmaceutical composition or device of claim 1, wherein the anti-apoptotic agent is Akt, an agonist of Akt, or a homologue or mimic thereof; Bre, an agonist of Bre, or a homologue or mimic thereof; erythropoietin, an agonist of erythropoietin, or a homologue or mimic thereof; fortilin, an agonist of fortilin, or a homologue or mimic thereof; a recombinant FNK protein (e.g., a FNK-TAT fusion protein); ghrelin, an agonist of ghrelin, or a homologue or mimic thereof; IAP (inhibitor of apoptosis protein), an agonist of IAP, or a homologue or mimic thereof; a PI3 kinase, an agonist of a PI3 kinase, or a homologue or mimic thereof; sirtuin, an agonist of sirtuin, or a homologue or mimic thereof; an inhibitor of the MAPK/JNK signaling cascade; an inhibitor of a member of the Bcl-2 family; an inhibitor of Fas; an inhibitor of NF-kB; an inhibitor of P38; an inhibitor of Ca²⁺ channels; an inhibitor of HO-1; an inhibitor of a caspase; an inhibitor of a calpain; p53; an inhibitor of the Src family of protein kinases; a trefoil factor, an agonist of a trefoil factor, or a homologue or mimic thereof; a heat shock protein, an agonist of a heat shock protein, or a homologue or mimic thereof; an apolipoprotein, an agonist of an apolipoprotein, or a homologue or mimic thereof; or combinations thereof.

8. The composition of claim 1, wherein the anti-apoptotic agent is AM-111.

9. The composition or device of claim 1, further comprising the anti-apoptotic agent, or pharmaceutically acceptable salt thereof, prodrug or combination thereof as an immediate release agent.

10. The pharmaceutical composition or device of claim 1, wherein the anti-apoptotic agent comprises multiparticulates.

11. The pharmaceutical composition or device of claim 1, wherein the anti-apoptotic agent is essentially in the form of micronized particles.

12. The pharmaceutical composition or device of claim 1, wherein the pH of the composition or device is between about 6.0 to about 7.6.

13. The pharmaceutical composition or device of claim 1, wherein the otic disease or condition is excitotoxicity, ototoxicity, presbycusis or combinations thereof.

14. A method of treating an otic disease or condition characterized by the apoptosis of a plurality of otic cells comprising: administering to an individual in need thereof an intratympanic composition or device comprising a therapeutically effective amount of the anti-apoptotic agent, the composition or device comprising substantially low degradation products of the anti-apoptotic agent, the composition or device further comprising two or more characteristics selected from:
(i) between about 0.1% to about 10% by weight of the anti-apoptotic agent, or pharmaceutically acceptable prodrug or salt thereof;
(ii) between about 14% to about 21% by weight of a polyoxyethylene-polyoxypropylene triblock copolymer of general formula E106 P70 E106;
(iii) sterile water, q.s., buffered to provide a pH between about 5.5 and about 8.0;
(iv) multiparticulate anti-apoptotic agent;
(v) a gelation temperature between about 19° C. to about 42° C.;
(vi) less than about 50 colony forming units (cfu) of microbiological agents per gram of delivery device;
(vii) less than about 5 endotoxin units (EU) per kg of body weight of a subject;
(viii) a mean dissolution time of about 30 hours for the; and
(ix) an apparent viscosity of about 100,000 cP to about 500,000 cP.

15. The method of claim 14, wherein the anti-apoptotic agent is released from the composition for a period of at least 3 days.

16. The method of claim 14, wherein the anti-apoptotic agent is essentially in the form of micronized particles.

17. The method of claim 14, wherein the anti-apoptotic agent inhibits apoptosis in a neuron or otic hair cell induced by glutamate activity.

18. The method of claim 14, wherein the anti-apoptotic agent inhibits apoptosis in a neuron or otic hair cell induced by an aminoglycoside antibiotic, a macrolide antibiotic, a glycopeptide antibiotic, salicylic acid, nicotine, actinomycin, bleomycin, cisplatin, carboplatin, vincristine, a loop diuretic, or combinations thereof.

19. The method of claim 14, wherein the anti-apoptotic agent inhibits apoptosis in an otic hair cell, a nerve cell, a cell of the stria vascularis induced by an accumulation of mutations in DNA, an accumulation of mutations in mitochondrial DNA, exposure to loud noise, infections, the lessening of blood flow to the ear, or combinations thereof.
